(19) United States
(12) Patent Application Publication
(10) Pub. No.: US 2021/0395704 A1
(43) Pub. Date: Dec. 23, 2021

(54) NANOBODY-GLYCAN MODIFYING ENZYME FUSION PROTEINS AND USES THEREOF

(71) Applicant: President and Fellows of Harvard College, Cambridge, MA (US)

(72) Inventors: Christina M. Woo, Cambridge, MA (US); Daniel Hector Ramirez, Somerville, MA (US); Chanat Aonbangkhen, Cambridge, MA (US)

(73) Assignee: President and Fellows of Harvard College, Cambridge, MA (US)

(21) Appl. No.: 17/289,613

(22) PCT Filed: Oct. 29, 2019
(86) PCT No.: PCT/US2019/058546

§ 371 Date: Apr. 28, 2021

Related U.S. Application Data
(60) Provisional application No. 62/752,186, filed on Oct. 29, 2018.

(51) Int. Cl. C12N 9/10; C12N 9/24; C07K 16/44; C07K 16/18

(52) U.S. Cl. CPC C12N 9/1051; C12N 9/2402; C07K 16/44; C07K 16/18; A61K 38/00; C12Y 204/01255; C07K 2319/60; C07K 2319/20; C07K 2317/569; C12Y 302/01169

(57) ABSTRACT

The present disclosure provides fusion proteins comprising a nanobody and a glycan modifying enzyme. Also provided herein are methods of glycosylating a protein and methods of removing a sugar from a protein. Further provided in the present disclosure are methods of treating and/or diagnosing diseases. Also provided herein are kits, polynucleotides, vectors, and cells.

### BACKGROUND OF INVENTION

Over 15% of the cellular proteome is modified by O-linked N-acetyl glucosamine (O-GlcNAc), a post-translational modification (PTM) that consists of a single glucosamine monosaccharide attached to serine or threonine residues of nuclear, cytosolic, and mitochondrial proteins. Due to the ubiquitous nature of the modification, O-GlcNAc has been implicated in numerous biological processes, including immune responses (Lund, P. J.; Elias, J. E.; Davis, M. M. Journal of Immunology (Baltimore, Md.: 1950) 2016, 197, 3086), cancer progression (Yi, W.; Clark, P. M.; Mason, D. E.; Keenan, M. C.; Hill, C.; Goddard III, W. A.; Peters, E. C.; Driggers, E. M.; Hsieh-Wilson, L. C. Science 2012, 337, 975), neurodegenerative diseases (Yuzwa, S. A.; Shan, X.; Macauley, M. S.; Clark, T.; Skorobogatko, Y.; Vosseller, K.; Vocadlo, D. J. Nature Chemical Biology 2012, 8, 393), and diabetes (Lagerlof, O.; Slocomb, J. E.; Hong, I.; Aponte, Y.; Blackshaw, S.; Hart, G. W.; Huganir, R. L. Science 2016, 351, 1293). The central role of 0-GlcNAc in cellular signaling is thought to derive from the metabolic link between O-GlcNAc and the hexosamine biosynthetic pathway (Butkinaree, C.; Park, K.; Hart, G. W. Biochimica et Biophysica Acta 2010, 1800, 96).

Despite a number of studies that point to the critical biological impact of O-GlcNAc on specific proteins, delineation of the function of O-GlcNAc modification on particular glycoproteins is hindered by the inability to control O-GlcNAc stoichiometry on specific proteins of interest in cells. Methods to increase or decrease global O-GlcNAc levels can be achieved through genetic manipulation or chemical inhibitors but are challenging to relate to the function of a specific glycoprotein (Gloster, T. M.; Zandberg, W. F.; Heinonen, J. E.; Shen, D. L.; Deng, L.; Vocadlo, D. J. Nature Chemical Biology 2011, 7, 174; Ortiz-Meoz, R. F.; Jiang, J.; Lazarus, M. B.; Orman, M.; Janetzko, J.; Fan, C.; Duveau, D. Y.; Tan, Z. W.; Thomas, C. J.; Walker, S. ACS Chemical Biology 2015, 10, 1392). Protein-specific manipulation of O-GlcNAc stoichiometry is possible by mutagenesis of transfected proteins to remove the glycosite or via total synthesis of the OGlcNAcylated protein in vitro (Marotta, N. P.; Lin, Y. H.; Lewis, Y. E.; Ambroso, M. R.; Zaro, B. W.; Roth, M. T.; Arnold, D. B.; Langen, R.; Pratt, M. R. Nature Chemistry 2015, 7, 913) These methods have defined specific functions for O-GlcNAc (Yi, W.; Clark, P. M.; Mason, D. E.; Keenan, M. C.; Hill, C.; Goddard III, W. A.; Peters, E. C.; Driggers, E. M.; Hsieh-Wilson, L. C. Science 2012, 337, 975) but prevent analysis of competing post-translational modification pathways (e.g., phosphorylation, ubiquitinylation), must be laboriously developed for every target protein, are challenging to implement for proteins carrying multiple glycosites, and are only possible if the exact glycosite is known. A general method to control glycosylation on specific target proteins would enable the systematic evaluation of OGlcNAc function in cells.

In contrast to other post-translational modifications, O-GlcNAc is installed and removed by only two enzymes: O-GlcNAc transferase (OGT) and O-GlcNAcase (OGA), which modify over 3,000 protein substrates (FIG. 1A) (Woo, C. M.; Lund, P. J.; Huang, A. C.; Davis, M. M.; Bertozzi, C. R.; Pitteri, S. J. Molecular & Cellular Proteomics 2018, 17, 764). O-GlcNAc is critical to cellular function as deletion of OGT in mice is embryonic lethal (Shafi, R.; Iyer, S. P.; Ellies, L. G.; O'Donnell, N.; Marek, K. W.; Chui, D.; Hart, G. W.; Marth, J. D. Proceedings of the National Academy of Sciences 2000, 97, 5735), deletion of OGA leads to perinatal death (Yang, Y. R.; Song, M.; Lee, H.; Jeon, Y.; Choi, E. J.; Jang, H. J.; Moon, H. Y.; Byun, H. Y.; Kim, E. K.; Kim, D. H.; Lee, M. N.; Koh, A.; Ghim, J.; Choi, J. H.; Lee-Kwon, W.; Kim, K. T.; Ryu, S. H.; Suh, P. G. Aging cell 2012, 11, 439), and conditional deletion of OGT in numerous cell types leads to senescence and apoptosis (O'Donnell, N.; Zachara, N. E.; Hart, G. W.; Marth, J. D. Molecular and Cellular Biology 2004, 24, 1680). OGT is a modular protein consisting of a catalytic domain connected to a tetratricopeptide repeat (TPR) domain that is thought to primarily direct substrate selection (Lazarus, M. B.; Nam, Y.; Jiang, J.; Sliz, P.; Walker, S. Nature 2011, 469, 564; Haltiwanger, R. S.; Blomberg, M. A.; Hart, G. W. The Journal of Biological Chemistry 1992, 267, 9005). OGA consists of a catalytic domain connected to a histone acetyltransferase (HAT)-like homology domain (Dong, D. L.; Hart, G. W. The Journal of Biological Chemistry 1994, 269, 19321). The parameters that dictate how these enzymes dynamically regulate thousands of O-GlcNAc modification sites on various substrates is still under investigation (Pathak, S.; Alonso, J.; Schimpl, M.; Rafie, K.; Blair, D. E.; Borodkin, V. S.; Schuttelkopf, A. W.; Albarbarawi, 0.; van Aalten, D. M. Nature Structural & Molecular Biology 2015, 22 (9), 744-50; Iyer, S. P. N.; Hart, G. W. The Journal of Biological Chemistry 2003, 278, 24608).

### SUMMARY OF INVENTION

Given the dynamic nature of O-GlcNAcylation and the large number of substrates modified by these two enzymes, it was hypothesized that controlling O-GlcNAc stoichiometry in a protein-specific manner could be achieved through proximity induction (FIG. 1B). Of the various mechanisms to induce protein—protein interactions, the controlled properties of nanobodies were particularly attractive. Nanobodies are small, highly-specific binding agents that are frequently used in affinity-based assays, imaging, X-ray crystallography, and recently as directing groups to recruit GFP (green fluorescent protein) fusion proteins for degradation (Caussinus, E.; Kanca, O.; Affolter, M. Nature Structural & Molecular Biology 2012, 19, 117; Dmitriev, O. Y.; Lutsenko, S.; Muyldermans, S. The Journal of Biological Chemistry 2016, 291, 3767).

Detailed herein is the development and use of proximity-directed nanobody-glycan modifying enzyme fusion proteins to systematically control glycan stoichiometry on specific target proteins in cells (FIG. 1C). Fusion of a nanobody to the N-terminus of OGT selectively controls O-GlcNAc stoichiometry on a series of tagged target proteins. Targeted induction of O-GlcNAc was achieved using a nanobody that recognizes GFP (nGFP, wherein “n” refers to nanobody, thus nGFP indicates a nanobody targeting GFP) and a nanobody that recognizes a four-amino acid sequence EPEA (nEPEA), and revealed induced 0-GlcNAcylation even with partial reduction of the TPR (tetratricopeptide repeat) domain which is primarily thought to direct substrate and glycosite selection. Proximity-directed OGT fusion proteins were additionally applied to elucidate whether the shift in subcellular localization of TET3 was due to the O-GlcNAc modification or association with OGT itself. The invention herein demonstrates a versatile platform for protein-specific OGlcNAcylation in live cells.

In one aspect, the present disclosure provides fusion proteins comprising a nanobody, or fragment thereof, connected to a glycan modifying enzyme via a linker. In another aspect, the present disclosure provides a polynucleotide encoding a fusion protein. In one aspect, the present disclosure provides a vector comprising a polynucleotide encoding a fusion protein. In another aspect, the present disclosure provides a cell comprising a fusion protein. In one aspect, the present disclosure provides a cell comprising the nucleic acid molecule encoding a fusion protein.

Also provided in the present disclosure are methods of use, which involve a fusion protein disclosed herein. In one aspect, the present disclosure provides a method of glycosylating a protein, the method comprising contacting a target protein with a fusion protein. In another aspect, the present disclosure provides a method of glycosylating a protein, the method comprising contacting a target protein with a fusion protein in the presence of a glycosyl donor molecule, thereby installing the sugar moiety from the glycosyl donor molecule on the target protein. In one aspect, the present disclosure provides a method of removing a sugar from a protein, the method comprising contacting a protein with a sugar moiety with a fusion protein, thereby excising the sugar moiety from the protein. In another aspect, the present disclosure provides a method of studying the effect of glycosylation in a cell using a fusion protein disclosed herein.

The present disclosure also provides methods of treating and diagnosing a subject. In one aspect, the present disclosure provides a method of treating a disease or disorder (e.g., neurodegenerative diseases (Parkinson's disease, Huntington's disease, Alzheimer's disease, demntia, multiple system atropy), psychotic disorders (e.g., schizophrenia), epilepsy, sleep disorders, and addictions), the method comprising administering a fusion protein to a subject in need thereof. In another aspect, the present disclosure provides a method of diagnosing a subject with a disease, the method comprising administering a fusion protein to the subject. In one aspect, the present disclosure provides a method of treating a subject suffering from or susceptible to a neurodegenerative disease, the method comprising administering an effective amount of a fusion protein to the subject. In another aspect, the present disclosure provides a method of treating a subject suffering from or susceptible to a psychotic disorder, the method comprising administering an effective amount of a fusion protein to the subject. In one aspect, the present disclosure provides a method of treating a subject suffering from or susceptible to epilepsy, the method comprising administering an effective amount of a fusion protein to the subject. In another aspect, the present disclosure provides a method of treating a subject suffering from or susceptible to a sleep disorder, the method comprising administering an effective amount of a fusion protein to the subject. In yet another aspect, the present disclosure provides a method of treating a subject suffering from or susceptible to an addiction, the method comprising administering an effective amount of a fusion protein to the subject.

Also provided herein are compositions, kits, polynucleotides, vectors, and cells. In one aspect, the present disclosure provides a pharmaceutical composition comprising a a fusion protrin and a pharmaceutically acceptable excipient. In another aspect, the present disclosure provides a kit comprising a fusion protein and an glycosyl donor molecule. In another aspect, the present disclosure provides a kit comprising a fusion protein and a glycosyl acceptor molecule. In one aspect, the present disclosure provides a polynucleotide encoding a fusion protein. In another aspect, the present disclosure provides a vector comprising a polynucleotide. In some aspects, the present disclosure provides a cell comprising a fusion protein. In another aspect, the present disclosure provides a cell compising a nucleic acid encoding a fusion protein.

The details of certain embodiments of the invention are set forth in the Detailed Description of Certain Embodiments, as described below. Other features, objects, and advantages of the invention will be apparent from the Definitions, Figures, Examples, and Claims.

### BRIEF DESCRIPTION OF THE DRAWINGS

FIG. 1A shows the structures of O-GlcNAc and GalNAz.

FIG. 1B shows a linear representation of the three major isoforms of OGT, ncOGT, mOGT, and sOGT.

FIG. 1C shows a strategy for selective induction of O-GlcNAc using a proximity-directed nanobody-OGT to transfer O-GlcNAc to the target protein.

FIG. 1D shows a linear representation of nanobody-OGT(13) and nanobody-OGT(4) fusion proteins.

FIG. 1E shows a general schematic of methods to detect O-GlcNAc stoichiometry on the target protein. HEK293T cells co-transfected with the target protein and OGT with or without the nanobody were incubated in the presence of Ac₄GalNAz as a reporter for O-GlcNAc. For O-GlcNAc protein quantification, cellular lysates were tagged with biotin-alkyne, affinity enriched with streptavidin-agarose, and the target protein visualized by Western blot or analyzed by mass spectrometry. For mass shift assays, cellular lysates were tagged with DBCO-PEG5K and visualized by Western blot.

FIG. 2A shows a linear representation of full length OGT(13), RFP (13), and nGFP(13). RFP (red fluorescent protein) and GFP (green fluorescent protein).

FIG. 2B shows subcellular localization of OGT (13), RFP(13), and nGFP(13) constructs expressed in HEK293T cells by confocal fluorescent microscopy. Scale bars represent 20 μm.

FIG. 2C shows a Western blot of O-GlcNAc levels on GFP-Flag-JunB-EPEA after immunoprecipitation with EPEA-beads from HEK293T cells. The expression of the various constructs was verified by Western blot analysis (10% input).

FIG. 2D shows a representation of the quantification of OGT expression. Data are representative of three biological replicates per experiment. Error bars represent standard deviation.

FIG. 2E shows a representation of the quantification of O-GlcNAc levels of GFP-Flag-JunB-EPEA after normalization to OGT expression. Data are representative of three biological replicates per experiment. Error bars represent standard deviation. * represents a p-value <0.05 under a two-tailed t-test.

FIG. 2F shows a representation of the quantification of O-GlcNAc levels in whole cell lysates. Data are representative of three biological replicates per experiment. Error bars represent standard deviation.

FIG. 3A shows a linear representation of TPR truncated OGT(4), RFP(4), nGFP(4), nEPEA(4), and catalytically inactive mutants.

FIG. 3B shows the subcellular localization of OGT(4), nGFP(4), and nEPEA(4) in HEK293T cells by confocal fluorescent microscopy. Scale bars represent 20 μm.

FIG. 3C shows a western blot and quantification of O-GlcNAc levels on GFP-Flag-JunB-EPEA after immunoprecipitation with EPEA-beads. The expression of the various constructs was verified by Western blot analysis (10% input). At least three biological replicates were performed per experiment. Error bars represent standard deviation, * represents a p-value <0.05, ** represents a p-value <0.01, *** represents a p-value <0.001, and **** represents a p-value <0.0001 under a two-tailed t-test or one-way ANOVA.

FIG. 3D shows a western blot and quantification of O-GlcNAc levels on GFP-Flag-JunB-EPEA after immunoprecipitation with EPEA-beads. The expression of the various constructs was verified by Western blot analysis (10% input). At least three biological replicates were performed per experiment. Error bars represent standard deviation, * represents a p-value <0.05, ** represents a p-value <0.01, *** represents a p-value <0.001, and **** represents a p-value <0.0001 under a two-tailed t-test or one-way ANOVA.

FIG. 3E shows a western blot and quantification of O-GlcNAc levels on JunB-Flag-EPEA after immunoprecipitation with EPEA-beads. The expression of the various constructs was verified by Western blot analysis (10% input). At least three biological replicates were performed per experiment. Error bars represent standard deviation, * represents a p-value <0.05, ** represents a p-value <0.01, *** represents a p-value <0.001, and **** represents a p-value <0.0001 under a two-tailed t-test or one-way ANOVA.

FIG. 3F shows a western blot and quantification of O-GlcNAc levels on GFP-Flag-JunB-EPEA after immunoprecipitation with EPEA-beads. The expression of the various constructs was verified by Western blot analysis (10% input). At least three biological replicates were performed per experiment. Error bars represent standard deviation, * represents a p-value <0.05, ** represents a p-value <0.01, *** represents a p-value <0.001, and **** represents a p-value <0.0001 under a two-tailed t-test or one-way ANOVA.

FIG. 3G shows a western blot and quantification of O-GlcNAc levels on JunB-Flag-EPEA, cJun-Flag-EPEA, and Nup62-Flag-EPEA after immunoprecipitation with EPEA-beads from α-syn KO HEK293 cells co-transfected with the indicated nanobody-OGT fusion protein and target protein. The expression of the various constructs was verified by Western blot analysis (10% input). At least three biological replicates were performed per experiment. Error bars represent standard deviation, * represents a p-value <0.05, ** represents a p-value <0.01, *** represents a p-value <0.001, and **** represents a p-value <0.0001 under a two-tailed t-test or one-way ANOVA.

FIG. 4A shows a Western blot of the target proteins Nup62, JunB, IKZF1, Zap70, and c-JUN after biotinylation and enrichment of azido-sugar labeled proteins. The target protein was cotransfected with HA-nEPEA-OGT(13) and metabolically labeled with Ac₄GalNAz in HEK293T cells.

FIG. 4B shows a Western blot of the target proteins Nup62, H2B, H3, c-JUN, JunB, and Zap70 after biotinylation and enrichment of azido-sugar labeled proteins. Endogenously O-GlcNAcylated CREB was visualized to represent any shifts in O-GlcNAc stoichiometry in the broader proteome. The target protein was co-transfected with HA-nEPEA-OGT(4) and metabolically labeled with Ac₄GalNAz in HEK293T cells.

FIG. 4C shows a mass shift assay for the degree of O-GlcNAc stoichiometry delivered by HA-OGT(4) or HA-nEPEA-OGT(4) fusions to target proteins c-JUN, H2B, H3, H4, and TET3. Cell lysates were treated with DBCO-PEG5K, heated at 90° C., and visualized by Western blot.

FIG. 4D shows a Western blot of OGT expression (anti-HA) from HEK293T cells co-transfected with the indicated target protein after mass shift assay. Cell lysates were treated with DBCO-PEG5K, heated at 95° C., and visualized by Western blot.

FIG. 4E shows a mass shift assay for the degree of O-GlcNAc stoichiometry delivered by HA-OGT(4) or HA-nEPEA-OGT(4) fusions to target proteins JunB, Zap70, Nup35, and STAT1 and endogenous O-GlcNAc protein CREB. Cell lysates were treated with DBCO-PEG5K, heated at 95° C., and visualized by Western blot.

FIG. 5A shows a representation of quantitative proteomics of enriched OGlcNAcylated proteins from α-syn KO HEK293 cells after co-expression of the indicated OGT construct (indicated as OGT) and JunB-Flag-EPEA (indicated as JunB).

FIG. 5B shows the glycopeptide and glycosite assignments of JunB-Flag-EPEA. The target protein was co-expressed with the indicated OGT fusion protein in α-syn KO HEK293 cells, immunoprecipitated, and analyzed by MS. X represent a glycosite observed under that condition. Only singly glycosylated peptides with unambiguous assignments and a PSM count >2 are given a glycosite designation. At least three biological replicates were performed per experiment. A JunB-Flag-EPEA glycosite overlap diagram is provided.

FIG. 5C shows the glycopeptide and glycosite assignments of Nup62-Flag-EPEA. The target protein was co-expressed with the indicated OGT fusion protein in α-syn KO HEK293 cells, immunoprecipitated, and analyzed by MS. X represent a glycosite observed under that condition. Only singly glycosylated peptides with unambiguous assignments and a PSM count >2 are given a glycosite designation. At least three biological replicates were performed per experiment. A Nup62-Flag-EPEA glycosite overlap diagram is provided.

FIG. 6A shows a Mass-shift assay workflow. O-GlcNAcylated cell lysates were chemoenzymatically labeled with GalNAz using GalT1. The GalNAz was reacted with a DBCOPEG5K and a western blot was performed to obtain an O-GlcNAc stoichiometry.

FIG. 6B shows a western blot and quantification of O-GlcNAc induced to α-synuclein by a mass shift assay. The indicated nanobody-OGT construct was expressed in HEK293T cells, the cells were lysed, chemoenzymatically labeled, and analyzed by mass shift assay. Global O-GlcNAc levels and the expression of the nanobody-OGT constructs was verified by Western blot analysis (10% input). At least six biological replicates were performed per experiment. Error bars represent standard deviation, ns represents p≥0.05, * represents p≤0.05, ** represents p≤0.01, **** represents p≤0.0001 under a two-tailed t-test or one-way ANOVA.

FIG. 7 shows subcellular localization of HEK293T cells transfected with pcDNA plasmid (control) or HA-nEPEA-OGT(4.5) via immunofluorescence.

FIG. 8 shows a Western blot for α-synuclein after separation of soluble and insoluble fractions with or without expression of HA-nEPEA-OGT(13), HA-nEPEA-OGT(4), or the TPR domain alone (HA-nEPEA-TPR).

FIG. 9 shows α-synuclein aggregates in U2OS cells with or without HA-nEPEA-OGT(4) via immunofluorescence.

FIG. 10 shows α-synuclein aggregates in HeLa cells with or without HA-nEPEA-OGT(4) via immunofluorescence.

### DEFINITIONS

Descriptions and certain information relating to various terms used in the present disclosure are collected herein for convenience.

As used herein and in the claims, the singular forms “a,” “an,” and “the” include the singular and the plural reference unless the context clearly indicates otherwise. Thus, for example, a reference to “an agent” includes a single agent and a plurality of such agents.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

A “nanobody,” as used herein, refers to a small protein recognition domain. Further, a nanobody is the smallest antigen binding fragment or single variable domain derived from naturally occurring heavy chain antibody and is known to the person skilled in the art. They are derived from heavy chain only antibodies, seen in camelids (Hamers-Casterman et al. 1993; Desmyter et al. 1996). In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe, and Lama vicugna). The single variable domain heavy chain antibody is herein designated as a nanobody or a VHH antibody. Nanobodies can also be derived from sharks.

The term “fusion protein,” as used herein, refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nanobody domain (e.g., a nanobody that directs the binding of the protein to a target site) and a glycan modifying enzyme. Any of the proteins provided herein may be produced by any method known in the art. For example, the proteins provided herein may be produced via recombinant protein expression and purification, which is especially suited for fusion proteins comprising a peptide linker or no linker. Methods for recombinant protein expression and purification are well known and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The terms “glycan,” “sugar,” “carbohydrate,” or “saccharide,” are used interchangeably herein and refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. An exemplary monosaccharide is O-linked N-acetylglucosamine (O-GlcNAc). Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 24) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, trehalose, and cellobiose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein. In some embodiments, the glycan is erythrose, threose, erythulose, arabinose, lyxose, ribose, xylose, ribulose, xylulose, allose, altrose, galactose, glucose, gulose, idose, mannose, talose, fructose, psicose, sorbose, tagatose, fucose, fuculose, rhamnose, mannoheptulose, sedoheptulose, and derivatives thereof (e.g., N-acetylglucosamine, N-acetylgalactosamine, etc.).

The term “glycosylation,” as used herein, is the reaction in which a glycosyl donor is attached to a functional group of a glycosyl acceptor. In some embodiments, glycosylation may refer to an enzymatic process that attaches glycans to proteins. In some embodiments, glycosylation may refer to an enzymatic process that attaches glycans to other glycans already attached to a protein. In some embodiments, glycosylation is the transfer of saccharide moieties to other molecules. In some embodiments, glycosylation refers to the modification of amino acids, such as serine and threonine, through their hydroxyl groups on proteins.

The term “glycosyl donor” as used herein is molecule that will donate a saccharide when reacted with a suitable glycosyl acceptor and form a new glycosidic bond. Exemplary glycosyl donors include uridine diphospho-D-glucose, uridine diphospho-D-galactose, uridine diphospho-D-xylose, uridine diphospho-N-acetyl-D-glucosamine, uridine diphospho-N-acetyl-D-galactosamine, uridine diphospho-D-glucuronic acid, uridine diphospho-D-galactofuranose, guanosine diphospho-D-mannose, guanosine diphospho-L-fucose, guanosine diphospho-L-rhamnose, cytidine monophospho-N-acetylneuraminic acid, and cytidine monophospho-2-keto-3-deoxy-D-mannooctanoic acid.

The term “glycosyl acceptor” as used herein is a suitable nucleophile-containing molecule that reacts with a glycosyl donor to form a new glycosidic bond. The nucleophile can be oxygen-, carbon-, nitrogen-, or sulfur-based. In certain embodiments, the nucleophile is —OH. In certain embodiments, the nucleophile is —NH₂ or —NHR.

The term “glycosidic bond,” as used herein, refers to a type of covalent bond that joins a carbohydrate to another group.

The term “kinase” is a type of enzyme that transfers phosphate groups from high energy donor molecules, such as ATP, to specific substrates, referred to as phosphorylation. Kinases are part of the larger family of phosphotransferases. One of the largest groups of kinases are protein kinases, which act on and modify the activity of specific proteins. Kinases are used extensively to transmit signals and control complex processes in cells. Various other kinases act on small molecules such as lipids, carbohydrates, amino acids, and nucleotides, either for signaling or to prime them for metabolic pathways. Kinases are often named after their substrates. More than 500 different protein kinases have been identified in humans. Exemplary human protein kinases include, but are not limited to, AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPR1A, BMPR1Aps1, BMPR1Aps2, BMPR1B, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDK2, CDK7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRK1A, DYRK1B, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK5, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST5, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLK1ps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Wee1, Wee1B, Wee1Bps, Wee1ps1, Wee1ps2, Wnk1, Wnk2, Wnk3, Wnk4, YANK1, YANK2, YANK5, YES, YESps, YSK1, ZAK, ZAP70, ZC1/HGK, ZC2/TNIK, ZC3/MINK, and ZC4/NRK.

A “transcription factor” is a type of protein that is involved in the process of transcribing DNA into RNA. Transcription factors can work independently or with other proteins in a complex to either stimulate or repress transcription. Transcription factors contain at least one DNA-binding domain that give them the ability to bind to specific sequences of DNA. Other proteins such as coactivators, chromatin remodelers, histone acetyltransferases, histone deacetylases, kinases, and methylases are also essential to gene regulation, but lack DNA-binding domains, and therefore are not transcription factors. These exemplary human transcription factors include, but are not limited to, AC008770.3, ACO23509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5L, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMP1, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK5, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FLI1, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE5, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES5, HES4, HES5, HES6, HEST, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT5, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERF1, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, ZZZ3.

The term “tetratricopeptide repeat” or “TPR” is a structural motif. The structural motif consists of a degenerate 34 amino acid sequence and is found in tandem arrays of 3-16 motifs, which mediate protein-protein interactions and assembly of multiprotein complexes. Alpha-helix pair repeats when folded together to produce a single, linear solenoid domain called a “tetratricopeptide repeat domain” or “TPR domain”.

“Click chemistry” is a chemical strategy introduced by Sharpless in 2001 and describes chemistry tailored to generate substances quickly and reliably by joining small units together. See, e.g., Kolb, Finn, and Sharpless, Angew Chem Int Ed 2001, 40, 2004; Evans, Australian Journal of Chemistry 2007, 60, 384. The term “click chemistry” does not refer to a specific reaction or set of reaction conditions, but instead refers to a class of reactions (e.g., coupling reactions). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; and Michael additions (e.g., maleimide addition). Examples of click chemistry reactions can be found in, e.g., Kolb, H. C.; Finn, M. G. and Sharpless, K. B. Angew. Chem. Int. Ed. 2001, 40, 2004; Kolb, H. C. and Sharpless, K. B. Drug Disc. Today 2003, 8, 112; Rostovtsev, V. V.; Green L. G.; Fokin, V. V. and Sharpless, K. B. Angew. Chem. Int. Ed. 2002, 41, 2596; Tomoe, C. W.; Christensen, C. and Meldal, M. J. Org. Chem. 2002, 67, 3057; Wang, Q. et al. J. Am. Chem. Soc. 2003, 125, 3192; Lee, L. V. et al. J. Am. Chem. Soc. 2003, 125, 9588; Lewis, W. G. et al. Angew. Chem. Int. Ed. 2002, 41, 1053; Manetsch, R. et al., J. Am. Chem. Soc. 2004, 126, 12809; Mocharla, V. P. et al. Angew. Chem. Int. Ed. 2005, 44, 116; each of which is incorporated by reference herein. In some embodiments, the click chemistry reaction involves a reaction with an alkyne moiety comprising a carbon-carbon triple bond (i.e., an alkyne handle). In some embodiments, the click chemistry reaction is a copper (I)-catalyzed azide-alkyne cycloaddition (CuAAC) reaction. A CuAAC reaction generates a 1,4-disubstituted-1,2,3-triazole product (i.e., a 5-membered heterocyclic ring). See, e.g., Hein J. E.; Fokin V. V. Chem Soc Rev, 2010, 39, 1302; which is incorporated herein by reference.

The term “sample” may be used to generally refer to an amount or portion of something (e.g., a protein). A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. The “sample” may be any biological sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments a sample comprises cells, tissue, or cellular material (e.g., material derived from cells, such as a cell lysate, or fraction thereof). A sample of a cell line comprises a limited number of cells of that cell line. In some embodiments, a sample may be obtained from an individual who has been diagnosed with or is suspected of having a disease.

The term “linker,” as used herein, refers to a bond (e.g., covalent bond), chemical group, or a molecule linking two molecules or moieties, e.g., two domains of a fusion protein, such as, for example, a nanobody domain and a glycan modifying domain (e.g., a glycan modifying enzyme). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making amino acid substitutions (mutations) are known in the art and are provided in, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single- and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The terms “condition,” “disease,” and “disorder” are used interchangeably.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; bbrain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

The term “psychotic disorders” is a subclass of psychiatric disorder refers to a disease of the mind and includes diseases and disorders listed in the Diagnostic and Statistical Manual of Mental Disorders-Fourth Edition (DSM-IV), published by the American Psychiatric Association, Washington D. C. (1994). Exemplary psychotic disorders include brief psychotic disorder, delusional disorder, schizoaffective disorder, schizophreniform disorder, schizophrenia, and shared psychotic disorder.

The term “addiction” is a brain disorder characterized by compulsive engagement in rewarding stimuli despite adverse consequences. Addiction may involve the use of substances such as alcohol, inhalants, opioids, cocaine, nicotine, and others, or behaviors such as gambling. Evidence suggests that the addictive substances and behaviors share a key neurobiological feature in that both intensely activate brain pathways of reward and reinforcement, many of which involve the neurotransmitter dopamine. Addiction is characterized by inability to consistently abstain, impairment in behavioral control, craving, diminished recognition of significant problems with one's behaviors and interpersonal relationships, and a dysfunctional emotional response.

The term “proteopathy” refers to a class of diseases in which certain proteins become structurally abnormal, and thereby disrupt the function of cells, tissues and organs of the body. Often the proteins fail to fold into their normal configuration; in this misfolded state, the proteins can become toxic in some way (e.g., a gain of toxic function) or they can lose their normal function.

The term “mis-fold” in relation to proteins refers to a case wherein a protein does not properly fold. The term “fold” in relation to proteins refers the physical process by which a protein chain acquires its native 3-dimensional structure, a conformation that is usually biologically functional, in an expeditious and reproducible manner. It is the physical process by which a polypeptide folds into its characteristic and functional three-dimensional structure from random coil. Each protein exists as an unfolded polypeptide or random coil when translated from a sequence of mRNA to a linear chain of amino acids. This polypeptide lacks any stable three-dimensional structure. As the polypeptide chain is being synthesized by a ribosome, the linear chain begins to fold into its three dimensional structure. Folding begins to occur even during translation of the polypeptide chain. Amino acids interact with each other to produce a well-defined three-dimensional structure, the folded protein, known as the native state. The resulting three-dimensional structure is determined by the amino acid sequence or primary structure. The energy landscape describes the folding pathways in which the unfolded protein is able to assume its native state. The correct three-dimensional structure is essential to function, although some parts of functional proteins may remain unfolded.

The term “aggregates” in relation to proteins refers to is a biological phenomenon in which mis-folded proteins aggregate (i.e., accumulate and clump together) either intra- or extracellularly. Protein aggregates are often correlated with diseases.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the inhibitor compound are outweighed by the therapeutically beneficial effects.

As used herein, “diagnostic agent” broadly refers to all agents capable of diagnosing a condition of interest.

As used herein, “therapeutic agent” broadly refers to all agents capable of treating a condition of interest. In one embodiment of the present invention, “therapeutic drug” may be a pharmaceutical composition comprising an effective ingredient and one or more pharmacologically acceptable carriers. A pharmaceutical composition can be manufactured, for example, by mixing an effective ingredient and the above-described carriers by any method known in the technical field of pharmaceuticals. Further, mode of usage of a therapeutic drug is not limited, as long as it is used for treatment. A therapeutic drug may be an effective ingredient alone or a mixture of an effective ingredient and any ingredient. Further, the type of the above-described carriers is not particularly limited.

“Contact,” “contacting,” and similar terms as used herein may refer to either direct or indirect contact, or both.

A “variant” of a particular polypeptide or polynucleotide has one or more additions, substitutions, and/or deletions with respect to the polypeptide or polynucleotide, which may be referred to as the “original polypeptide” or “original polynucleotide,” respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments, a fragment or variant is at least 20%, 30%, 40%, 50%, 60%, 70%, 80%, 90%, 95%, 96%, 97%, 98%, 99%, or more as long as the original polypeptide or polynucleotide.

In some embodiments a variant is a biologically active variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological structure or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments, a variant, e.g., a biologically active variant, comprises or consists of a polypeptide at least 95%, 96%, 97%, 98%, 99%, 99.5%, or 100% identical to an original polypeptide over at least 70%, 75%, 80%, 85%, 90%, 95%, 96%, 97%, 98%, 99%, or 100% of the original polypeptide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. Variants may be tested in one or more suitable assays to assess activity.

As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. In certain embodiments, an antibody is an antibody fragment. However, in some embodiments, an antibody is a Fab fragment, a F(ab′)2 fragment, a Fv fragment, or a scFv fragment. In some embodiments, an antibody is a nanobody derived from a camelid antibody or a nanobody derived from a shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ), or mu (μ) heavy chain. In some embodiments, the heavy chain of an antibody described herein comprises a human alpha (α), delta (Δ), epsilon (ε), gamma (γ), or mu (μ) heavy chain. In a particular embodiment, an antibody described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780. In some embodiments, the VH domain comprises an amino acid sequence that is at least 70%, 75%, 80%, 85%, 90%, 95%, 98%, or at least 99% identical to any of the variable chain constant regions. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger et al., Proceedings of the National Academy of Sciences 1993, 90, 6444; Poljak et al., Structure 1994, 2, 1121).

### DETAILED DESCRIPTION OF CERTAIN EMBODIMENTS

The aspects described herein are not limited to specific embodiments, methods, or configurations, and as such can, of course, vary. The terminology used herein is for the purpose of describing particular aspects only and, unless specifically defined herein, is not intended to be limiting.

The present disclosure provides fusion proteins comprising a nanobody and a glycan modifying enzyme (e.g., enzyme involved in glycan transformations, including adding, removing, or altering a glycan). Also provided herein are methods of glycosylating a protein and methods of removing a sugar from a protein using a fusion protein as described herein. Further provided in the present disclosure are methods and uses of treating and/or diagnosing diseases using the fusion proteins described herein. Also provided herein are kits, polynucleotides encoding the fusion proteins or domain thereof, vectors comprising such polynucleotides, and cells comprising such polynucleotides or vectors.

The present disclosure provides fusion proteins allowing for the specific and directed modification of target proteins either by introduction or removal of a glycan, thus altering the molecular structure of the target proteins. In certain embodiments, the change in molecular structure results in conformational changes. In certain embodiments, these changes in structure and conformation have implications regarding the functions and interactions of the protein. In some aspects, the introduction or removal of a glycan will impact the ability of the protein to form aggregates, which are often correlated in diseases.

### Fusion Proteins

In certain embodiments, the fusion protein comprises a nanobody and a glycan modifying enzyme. In some embodiments, the nanobody and glycan modifying enzyme are connected via a linker consisting of a short peptide sequence. In certain embodiments, the nanobody is fused to the N-terminal domain of the enzyme. In other embodiments, the nanobody is fused to the C-terminus of the enzyme.

In certain embodiments, the glycan modifying enzyme of the fusion protein is a glycosyl transferase. A glycosyl transferase is a type of enzyme that catalyzes the formation of the glycosidic linkage by transferring a glycosyl donor molecule to an glycosyl acceptor. In some embodiments, the only a fragment of a glycosyl transferase is used in the fusion protein. In some embodiments, a variant of a glycosyl transferase is used in the fusion protein. In some embodiments, only certain domains of a glycosyl transferase is used in the fusion protein. In some embodiments, the glycosyl transferase is a hexosyltransferase. In certain embodiments, the glycan modifying enzyme is O-GlcNAc transferase. In certain embodiments, the glycan modifying enzyme is galactoside 3-L-fucosyltransferase (Fut9). In certain embodiments, the glycan modifying enzyme O-fucosyltransferase SPY. Exemplary glycosyl transferases include glycogen phosphorylase, dextrin dextranase, amylosucrase, dextransucrase, sucrose phosphorylase, maltose phosphorylase, inulosucrase, levansucrase, glycogen(starch) synthase, cellulose synthase (UDP-forming), sucrose synthase, sucrose-phosphate synthase, α,α-trehalose-phosphate synthase (UDP-forming), chitin synthase, glucuronosyltransferase, 1,4-α-glucan branching enzyme, cyclomaltodextrin glucanotransferase, cellobiose phosphorylase, starch synthase (glycosyl-transferring), lactose synthase, sphingosine β-galactosyltransferase, 1,4-α-glucan 6-α-glucosyltransferase, 4-α-glucanotransferase, DNA α-glucosyltransferase, DNA β-glucosyltransferase, glucosyl-DNA β-glucosyltransferase, cellulose synthase (GDP-forming), 1,3-β-oligoglucan phosphorylase, laminaribiose phosphorylase, glucomannan 4-β-mannosyltransferase, mannuronan synthase, 1,3-β-glucan synthase, phenol β-glucosyltransferase, α,α-trehalose-phosphate synthase (GDP-forming), fucosylglycoprotein 3-α-galactosyltransferase, β-N-acetylglucosaminylglycopeptide β-1,4-galactosyltransferase, steroid N-acetylglucosaminyltransferase, fucosylgalactose α-N-acetylgalactosaminyltransferase, polypeptide N-acetylgalactosaminyltransferase, polygalacturonate 4-α-galacturonosyltransferase, lipopolysaccharide 3-α-galactosyltransferase, monogalactosyldiacylglycerol synthase, N-acylsphingosine galactosyltransferase, heteroglycan α-mannosyltransferase, cellodextrin phosphorylase, procollagen galactosyltransferase, poly(glycerol-phosphate) α-glucosyltransferase, poly(ribitol-phosphate) β-glucosyltransferase, undecaprenyl-phosphate mannosyltransferase, lipopolysaccharide N-acetylglucosaminyltransferase, lipopolysaccharide glucosyltransferase I, abequosyltransferase, ganglioside galactosyltransferase, linamarin synthase, α,α-trehalose phosphorylase, 3-galactosyl-N-acetylglucosaminide 4-α-L-fucosyltransferase, procollagen glucosyltransferase, galactinol-raffinose galactosyltransferase, glycoprotein 6-α-L-fucosyltransferase, type 1 galactoside α-(1,2)-fucosyltransferase, poly(ribitol-phosphate) α-N-acetylglucosaminyltransferase, arylamine glucosyltransferase, lipopolysaccharide glucosyltransferase II, glycosaminoglycan galactosyltransferase, phosphopolyprenol glucosyltransferase, globotriaosylceramide 3-β-N-acetylgalactosaminyltransferase, ceramide glucosyltransferase, flavone 7-O-β-glucosyltransferase, galactinol-sucrose galactosyltransferase, dolichyl-phosphate β-D-mannosyltransferase, cyanohydrin β-glucosyltransferase, N-acetyl-β-D-glucosaminide β-(1,3)-galactosyltransferase, N-acetyllactosaminide 3-α-galactosyltransferase, globoside α-N-acetylgalactosaminyltransferase, N-acetyllactosamine synthase, flavonol 3-O-glucosyltransferase, (N-acetylneuraminyl)-galactosylglucosylceramide N-acetylgalactosaminyltransferase, protein N-acetylglucosaminyltransferase, sn-glycerol-3-phosphate 1-galactosyltransferase, 1,3-β-D-glucan phosphorylase, sucrose:sucrose fructosyltransferase, 2,1-fructan:2,1-fructan 1-fructosyltransferase, α-1,3-mannosyl-glycoprotein 2-β-N-acetylglucosaminyltransferase, β-1,3-galactosyl-O-glycosyl-glycoprotein β-1,6-N-acetylglucosaminyltransferase, alizarin 2-β-glucosyltransferase, o-dihydroxycoumarin 7-O-glucosyltransferase, vitexin β-glucosyltransferase, isovitexin β-glucosyltransferase, dolichyl-phosphate-mannose-protein mannosyltransferase, tRNA-queuosine β-mannosyltransferase, coniferyl-alcohol glucosyltransferase, α-1,4-glucan-protein synthase (ADP-forming), 2-coumarate O-β-glucosyltransferase, anthocyanidin 3-O-glucosyltransferase, cyanidin 3-O-rutinoside 5-O-glucosyltransferase, dolichyl-phosphate β-glucosyltransferase, cytokinin 7-β-glucosyltransferase, sinapate 1-glucosyltransferase, indole-3-acetate β-glucosyltransferase, N-acetylgalactosaminide β-1,3-galactosyltransferase, inositol 3-α-galactosyltransferase, sucrose-1,6-α-glucan 3(6)-α-glucosyltransferase, hydroxycinnamate 4-β-glucosyltransferase, monoterpenol β-glucosyltransferase, scopoletin glucosyltransferase, peptidoglycan glycosyltransferase, dolichyl-phosphate-mannose-glycolipid α-mannosyltransferase, GDP-Man:Man3GlcNAc2-PP-dolichol α-1,2-mannosyltransferase, GDP-Man:Man1GlcNAc2-PP-dolichol α-1,3-mannosyltransferase, xylosylprotein 4-β-galactosyltransferase, galactosylxylosylprotein 3-β-galactosyltransferase, galactosylgalactosylxylosylprotein 3-β-glucuronosyltransferase, gallate 1-β-glucosyltransferase, sn-glycerol-3-phosphate 2-α-galactosyltransferase, mannotetraose 2-α-N-acetylglucosaminyltransferase, maltose synthase, alternansucrase, N-acetylglucosaminyldiphosphodolichol N-acetylglucosaminyltransferase, chitobiosyldiphosphodolichol α-mannosyltransferase, α-1,6-mannosyl-glycoprotein 2-β-N-acetylglucosaminyltransferase, β-1,4-mannosyl-glycoprotein 4-β-N-acetylglucosaminyltransferase, α-1,3-mannosyl-glycoprotein 4-β-N-acetylglucosaminyltransferase, β-1,3-galactosyl-O-glycosyl-glycoprotein β-1,3-N-acetylglucosaminyltransferase, acetylgalactosaminyl-O-glycosyl-glycoprotein β-1,3-N-acetylglucosaminyltransferase, acetylgalactosaminyl-O-glycosyl-glycoprotein β-1,6-N-acetylglucosaminyltransferase, N-acetyllactosaminide β-1,3-N-acetylglucosaminyltransferase, N-acetyllactosaminide β-1,6-N-acetylglucosaminyltransferase, galactoside 3-fucosyltransferase, UDP-N-acetylglucosamine-dolichyl-phosphate N-acetylglucosaminyltransferase, α-1,6-mannosyl-glycoprotein 6-β-N-acetylglucosaminyltransferase, indolylacetyl-myo-inositol galactosyltransferase, 13-hydroxydocosanoate 13-β-glucosyltransferase, flavonol-3-O-glucoside L-rhamnosyltransferase, pyridoxine 5′-O-β-D-glucosyltransferase, oligosaccharide 4-α-D-glucosyltransferase, aldose β-D-fructosyltransferase, N-acetylneuraminylgalactosylglucosylceramide β-1,4-N-acetylgalactosaminyltransferase, raffinose-raffinose α-galactosyltransferase, sucrose 6F-α-galactosyltransferase, xyloglucan 4-glucosyltransferase, isoflavone 7-O-glucosyltransferase, methyl-ONN-azoxymethanol β-D-glucosyltransferase, salicyl-alcohol β-D-glucosyltransferase, sterol 3β-glucosyltransferase, glucuronylgalactosylproteoglycan 4-β-N-acetylgalactosaminyltransferase, glucuronosyl-N-acetylgalactosaminyl-proteoglycan 4-β-N-acetylgalactosaminyltransferase, gibberellin β-D-glucosyltransferase, cinnamate β-D-glucosyltransferase, hydroxymandelonitrile glucosyltransferase, lactosylceramide β-1,3-galactosyltransferase, lipopolysaccharide N-acetylmannosaminouronosyltransferase, hydroxyanthraquinone glucosyltransferase, lipid-A-disaccharide synthase, α-1,3-glucan synthase, galactolipid galactosyltransferase, flavanone 7-O-β-glucosyltransferase, glycogenin glucosyltransferase, N-acetylglucosaminyldiphosphoundecaprenol N-acetyl-β-D-mannosaminyltransferase, N-acetylglucosaminyldiphosphoundecaprenol glucosyltransferase, luteolin 7-O-glucuronosyltransferase, luteolin-7-O-glucuronide 2″-O-glucuronosyltransferase, luteolin-7-O-diglucuronide 4′-O-glucuronosyltransferase, nuatigenin 3β-glucosyltransferase, sarsapogenin 3β-glucosyltransferase, 4-hydroxybenzoate 4-O-β-D-glucosyltransferase, N-hydroxythioamide S-β-glucosyltransferase, nicotinate glucosyltransferase, high-mannose-oligosaccharide β-1,4-N-acetylglucosaminyltransferase, phosphatidylinositol N-acetylglucosaminyltransferase, β-mannosylphosphodecaprenol-mannooligosaccharide 6-mannosyltransferase, α-1,6-mannosyl-glycoprotein 4-β-N-acetylglucosaminyltransferase, 2,4-dihydroxy-7-methoxy-2H-1,4-benzoxazin-3(4H)-one 2-D-glucosyltransferase, zeatin O-β-D-glucosyltransferase, galactogen 6β-galactosyltransferase, lactosylceramide 1,3-N-acetyl-β-D-glucosaminyltransferase, xyloglucan:xyloglucosyl transferase, diglucosyl diacylglycerol synthase (1,2-linking), cis-p-coumarate glucosyltransferase, limonoid glucosyltransferase, 1,3-β-galactosyl-N-acetylhexosamine phosphorylase, hyaluronan synthase, glucosylglycerol-phosphate synthase, glycoprotein 3-α-L-fucosyltransferase, cis-zeatin O-β-D-glucosyltransferase, trehalose 6-phosphate phosphorylase, mannosyl-3-phosphoglycerate synthase, hydroquinone glucosyltransferase, vomilenine glucosyltransferase, indoxyl-UDPG glucosyltransferase, peptide-O-fucosyltransferase, O-fucosylpeptide 3-β-N-acetylglucosaminyltransferase, glucuronyl-galactosyl-proteoglycan 4-α-N-acetylglucosaminyltransferase, glucuronosyl-N-acetylglucosaminyl-proteoglycan 4-α-N-acetylglucosaminyltransferase, N-acetylglucosaminyl-proteoglycan 4-β-glucuronosyltransferase, N-acetylgalactosaminyl-proteoglycan 3-β-glucuronosyltransferase, undecaprenyldiphospho-muramoylpentapeptide β-N-acetylglucosaminyltransferase, lactosylceramide 4-α-galactosyltransferase, [Skp1-protein]-hydroxyproline N-acetylglucosaminyltransferase, kojibiose phosphorylase, α,α-trehalose phosphorylase (configuration-retaining), glycolipid 6-α-mannosyltransferase, kaempferol 3-O-galactosyltransferase, cyanidin 3-O-rutinoside 5-O-glucosyltransferase, flavanone 7-O-glucoside 2″-O-β-L-rhamnosyltransferase, flavonol 7-O-β-glucosyltransferase, delphinidin 3,5-di-O-glucoside 3′-O-glucosyltransferase, flavonol-3-O-glucoside glucosyltransferase, flavonol-3-O-glycoside glucosyltransferase, digalactosyldiacylglycerol synthase, NDP-glucose-starch glucosyltransferase, 6G-fructosyltransferase, N-acetyl-β-glucosaminyl-glycoprotein 4-O—N-acetylgalactosaminyltransferase, α,α-trehalose synthase, mannosylfructose-phosphate synthase, β-D-galactosyl-(1→4)-L-rhamnose phosphorylase, cycloisomaltooligosaccharide glucanotransferase, delphinidin 3′,5′-O-glucosyltransferase, D-inositol-3-phosphate glycosyltransferase, GlcA-β-(1→2)-D-Man-α-(1→3)-D-Glc-β-(1→4)-D-Glc-α-1-diphosphoundecaprenol 4-O-mannosyltransferase, GDP-mannose:cellobiosyl-diphosphopolyprenol α-mannosyltransferase, baicalein 7-O-glucuronosyltransferase, cyanidin-3-O-glucoside 2-O-glucuronosyltransferase, protein O-GlcNAc transferase, dolichyl-P-Glc:Glc2Man9GlcNAc2-PP-dolichol α-1,2-glucosyltransferase, GDP-Man:Man2GlcNAc2-PP-dolichol α-1,6-mannosyltransferase, dolichyl-P-Man:Man5GlcNAc2-PP-dolichol α-1,3-mannosyltransferase, dolichyl-P-Man:Man6GlcNAc2-PP-dolichol α-1,2-mannosyltransferase, dolichyl-P-Man:Man7GlcNAc2-PP-dolichol α-1,6-mannosyltransferase, dolichyl-P-Man:Man8GlcNAc2-PP-dolichol α-1,2-mannosyltransferase, soyasapogenol glucuronosyltransferase, abscisate β-glucosyltransferase, D-Man-α-(1→3)-D-Glc-β-(1→4)-D-Glc-α-1-diphosphoundecaprenol 2-O-glucuronyltransferase, dolichyl-P-Glc:Glc1Man9GlcNAc2-PP-dolichol α-1,3-glucosyltransferase, glucosyl-3-phosphoglycerate synthase, dolichyl-P-Glc:Man9GlcNAc2-PP-dolichol α-1,3-glucosyltransferase, glucosylglycerate synthase, mannosylglycerate synthase, mannosylglucosyl-3-phosphoglycerate synthase, crocetin glucosyltransferase, soyasapogenol B glucuronide galactosyltransferase, soyasaponin III rhamnosyltransferase, glucosylceramide β-1,4-galactosyltransferase, neolactotriaosylceramide β-1,4-galactosyltransferase, zeaxanthin glucosyltransferase, 10-deoxymethynolide desosaminyltransferase, 3-α-mycarosylerythronolide B desosaminyl transferase, nigerose phosphorylase, N,N′-diacetylchitobiose phosphorylase, 4-O-β-D-mannosyl-D-glucose phosphorylase, 3-O-α-D-glucosyl-L-rhamnose phosphorylase, 2-deoxystreptamine N-acetyl-D-glucosaminyltransferase, 2-deoxystreptamine glucosyltransferase, UDP-GlcNAc:ribostamycin N-acetylglucosaminyltransferase, chalcone 4′-O-glucosyltransferase, rhamnopyranosyl-N-acetylglucosaminyl-diphospho-decaprenol β-1,4/1,5-galactofuranosyltransferase, galactofuranosylgalactofuranosylrhamnosyl-N-acetylglucosaminyl-diphospho-decaprenol β-1,5/1,6-galactofuranosyltransferase, N-acetylglucosaminyl-diphospho-decaprenol L-rhamnosyltransferase, N,N′-diacetylbacillosaminyl-diphospho-undecaprenol α-1,3-N-acetylgalactosaminyltransferase, N-acetylgalactosamine-N,N′-diacetylbacillosaminyl-diphospho-undecaprenol 4-α-N-acetylgalactosaminyltransferase, GalNAc-α-(1→4)-GalNAc-α-(1→3)-diNAcBac-PP-undecaprenol α-1,4-N-acetyl-D-galactosaminyltransferase, GalNAc5-diNAcBac-PP-undecaprenol β-1,3-glucosyltransferase, cyanidin 3-O-galactosyltransferase, anthocyanin 3-O-sambubioside 5-O-glucosyltransferase, anthocyanidin 3-O-coumaroylrutinoside 5-O-glucosyltransferase, anthocyanidin 3-O-glucoside 2″-O-glucosyltransferase, anthocyanidin 3-O-glucoside 5-O-glucosyltransferase, cyanidin 3-O-glucoside 5-O-glucosyltransferase (acyl-glucose), cyanidin 3-O-glucoside 7-O-glucosyltransferase (acyl-glucose), 2′-deamino-2′-hydroxyneamine 1-α-D-kanosaminyltransferase, L-demethylnoviosyl transferase, UDP-Gal:α-D-GlcNAc-diphosphoundecaprenol β-1,3-galactosyltransferase, UDP-Gal: α-D-GlcNAc-diphosphoundecaprenol β-1,4-galactosyltransferase, UDP-Glc:α-D-GlcNAc-glucosaminyl-diphosphoundecaprenol β-1,3-glucosyltransferase, UDP-GalNAc: α-D-GalNAc-diphosphoundecaprenol α-1,3-N-acetylgalactosaminyltransferase, GDP-Fuc:β-D-Gal-1,3-α-D-GalNAc-1,3-α-GalNAc-diphosphoundecaprenol α-1,2-fucosyltransferase, UDP-Gal:α-L-Fuc-1,2-O-Gal-1,3-α-GalNAc-1,3-α-GalNAc-diphosphoundecaprenol α-1,3-galactosyltransferase, vancomycin aglycone glucosyltransferase, chloroorienticin B synthase, protein O-mannose β-1,4-N-acetylglucosaminyltransferase, protein O-mannose β-1,3-N-acetylgalactosaminyltransferase, ginsenoside Rd glucosyltransferase, diglucosyl diacylglycerol synthase (1,6-linking), tylactone mycaminosyltransferase, O-mycaminosyltylonolide 6-deoxyallosyltransferase, demethyllactenocin mycarosyltransferase, β-1,4-mannooligosaccharide phosphorylase, 1,4-β-mannosyl-N-acetylglucosamine phosphorylase, cellobionic acid phosphorylase, desvancosaminyl-vancomycin vancosaminetransferase, 7-deoxyloganetic acid glucosyltransferase, 7-deoxyloganetin glucosyltransferase, TDP-N-acetylfucosamine:lipid II N-acetylfucosaminyltransferase, aklavinone 7-O-L-rhodosaminyltransferase, aclacinomycin-T 2-deoxy-L-fucose transferase, erythronolide mycarosyltransferase, sucrose 6F-phosphate phosphorylase, β-D-glucosyl crocetin β-1,6-glucosyltransferase, 8-demethyltetracenomycin C L-rhamnosyltransferase, 1,2-α-glucosylglycerol phosphorylase, 1,2-β-oligoglucan phosphorylase, 1,3-α-oligoglucan phosphorylase, dolichyl N-acetyl-α-D-glucosaminyl phosphate 3-O-D-2,3-diacetamido-2,3-dideoxy-β-D-glucuronosyltransferase, monoglucosyldiacylglycerol synthase, 1,2-diacylglycerol 3-α-glucosyltransferase, validoxylamine A glucosyltransferase, β-1,2-mannobiose phosphorylase, 1,2-β-oligomannan phosphorylase, α-1,2-colitosyltransferase, α-maltose-1-phosphate synthase, UDP-Gal:α-D-GlcNAc-diphosphoundecaprenol α-1,3-galactosyltransferase, type 2 galactoside α-(1,2)-fucosyltransferase, phosphatidyl-myo-inositol α-mannosyltransferase, phosphatidyl-myo-inositol dimannoside synthase, α,α-trehalose-phosphate synthase (ADP-forming), N-acetyl-α-D-glucosaminyl-diphospho-ditrans, octacis-undecaprenol 3-α-mannosyltransferase, mannosyl-N-acetyl-α-D-glucosaminyl-diphospho-ditrans, octacis-undecaprenol 3-α-mannosyltransferase, mogroside IE synthase, rhamnogalacturonan I rhamnosyltransferase, glucosylglycerate phosphorylase, sordaricin 6-deoxyaltrosyltransferase, (R)-mandelonitrile β-glucosyltransferase, poly(ribitol-phosphate) β-N-acetylglucosaminyltransferase, glucosyl-dolichyl phosphate glucuronosyltransferase, phlorizin synthase, and acylphloroglucinol glucosyltransferase.

In other embodiments, the glycosyltransferase is a pentosyltransferase. Exemplary pentosyltranferases include purine-nucleoside phosphorylase, pyrimidine-nucleoside phosphorylase, uridine phosphorylase, thymidine phosphorylase, nucleoside ribosyltransferase, nucleoside deoxyribosyltransferase, adenine phosphoribosyltransferase, hypoxanthine phosphoribosyltransferase, uracil phosphoribosyltransferase, orotate phosphoribosyltransferase, nicotinate phosphoribosyltransferase, nicotinamide phosphoribosyltransferase, amidophosphoribosyltransferase, guanosine phosphorylase, urate-ribonucleotide phosphorylase, ATP phosphoribosyltransferase, anthranilate phosphoribosyltransferase, nicotinate-nucleotide diphosphorylase (carboxylating), dioxotetrahydropyrimidine phosphoribosyltransferase, nicotinate-nucleotide-dimethylbenzimidazole phosphoribosyltransferase, xanthine phosphoribosyltransferase, 1,4-3-D-xylan synthase, flavone apiosyltransferase, protein xylosyltransferase, dTDP-dihydrostreptose-streptidine-6-phosphate dihydrostreptosyltransferase, S-methylthio-5′-adenosine phosphorylase, tRNA-guanosine34 transglycosylase, NAD+ ADP-ribosyltransferase, NAD⁺-protein-arginine ADP-ribosyltransferase, dolichyl-phosphate D-xylosyltransferase, dolichyl-xylosyl-phosphate-protein xylosyltransferase, indolylacetylinositol arabinosyltransferase, flavonol-3-O-glycoside xylosyltransferase, NAD⁺-diphthamide ADP-ribosyltransferase, NAD⁺-dinitrogen-reductase ADP-D-ribosyltransferase, glycoprotein 2-β-D-xylosyltransferase, xyloglucan 6-xylosyltransferase, zeatin O-β-D-xylosyltransferase, xylogalacturonan β-1,3-xylosyltransferase, UDP-D-xylose:β-D-glucoside α-1,3-D-xylosyltransferase, lipid IVA 4-amino-4-deoxy-L-arabinosyltransferase, S-methyl-5′-thioinosine phosphorylase, decaprenyl-phosphate phosphoribosyltransferase, galactan 5-O-arabinofuranosyltransferase, arabinofuranan 3-O-arabinosyltransferase, tRNA-guanine15 transglycosylase, neamine phosphoribosyltransferase, cyanidin 3-O-galactoside 2″-O-xylosyltransferase, anthocyanidin 3-O-glucoside 2′″-O-xylosyltransferase, triphosphoribosyl-dephospho-CoA synthase, undecaprenyl-phosphate 4-deoxy-4-formamido-L-arabinose transferase, (3-ribofuranosylaminobenzene 5′-phosphate synthase, nicotinate D-ribonucleotide:phenol phospho-D-ribosyltransferase, kaempferol 3-O-xylosyltransferase, AMP phosphorylase, hydroxyproline 0-arabinosyltransferase, sulfide-dependent adenosine diphosphate thiazole synthase, and cysteine-dependent adenosine diphosphate thiazole synthase.

In other embodiments, the glycosyltransferase is selected from the group consisting of β-galactoside α-2,6-sialyltransferase, β-D-galactosyl-(1→3)-N-acetyl-β-D-galactosaminide α-2,3-sialyltransferase, α-N-acetylgalactosaminide α-2,6-sialyltransferase, β-galactoside α-2,3-sialyltransferase, galactosyldiacylglycerol α-2,3-sialyltransferase, N-acetyllactosaminide α-2,3-sialyltransferase, (α-N-acetylneuraminyl-2,3-β-galactosyl-1,3)-N-acetyl-galactosaminide 6-α-sialyltransferase, α-N-acetylneuraminate α-2,8-sialyltransferase, lactosylceramide α-2,3-sialyltransferase, lipid IVA 3-deoxy-D-manno-octulosonic acid transferase, (KDO)-lipid IVA 3-deoxy-D-manno-octulosonic acid transferase, (KDO)2-lipid IVA (2-8) 3-deoxy-D-manno-octulosonic acid transferase, (KDO)3-lipid IVA (2-4) 3-deoxy-D-manno-octulosonic acid transferase, starch synthase (maltosyl-transferring), S-adenosylmethionine:tRNA ribosyltransferase-isomerase, dolichyl-diphosphooligosaccharide-protein glycotransferase, undecaprenyl-diphosphooligosaccharide-protein glycotransferase 2′-phospho-ADP-ribosyl cyclase/2′-phospho-cyclic-ADP-ribose transferase, and dolichyl-phosphooligosaccharide-protein glycotransferase.

In certain embodiments, the enzyme is a glycosyl hydrolase. A glycosyl hydrolase is a type of enzyme that catalyzes the hydrolysis of a glycosidic bond by excising a glycan to an glycosyl acceptor. In some embodiments, the only a fragment of a glycosyl hydrolase is used in the fusion protein. In some embodiments, a variant of a glycosyl hydrolase is used in the fusion protein. In some embodiments, only certain domains of a glycosyl hydrolase is used in the fusion protein. In certain embodiments, the enzyme is O-GlcNAcase (OGA). Exemplary glycosyl hydrolases include α-amylase, β-amylase, glucan 1,4-α-glucosidase, cellulase, endo-1,3(4)-β-glucanase, inulinase, endo-1,4-β-xylanase, oligo-1,6-glucosidase, dextranase, chitinase, polygalacturonase, lysozyme, exo-α-sialidase, α-glucosidase, β-glucosidase, α-galactosidase, β-galactosidase, α-mannosidase, β-mannosidase, β-fructofuranosidase, α,α-trehalase, 3-glucuronidase, endo-1,3-β-xylanase, amylo-1,6-glucosidase, hyaluronoglucosaminidase, hyaluronoglucuronidase, xylan 1,4-β-xylosidase, (3-D-fucosidase, glucan endo-1,3-β-D-glucosidase, α-L-rhamnosidase, pullulanase, GDP-glucosidase, β-L-rhamnosidase, fucoidanase, glucosylceramidase, galactosylceramidase, galactosylgalactosylglucosylceramidase, sucrose α-glucosidase, α-N-acetylgalactosaminidase, α-N-acetylglucosaminidase, α-L-fucosidase, β-L-N-acetylhexosaminidase, β-N-acetylgalactosaminidase, cyclomaltodextrinase, non-reducing end α-L-arabinofuranosidase, glucuronosyl-disulfoglucosamine glucuronidase, isopullulanase, glucan 1,3-β-glucosidase, glucan endo-1,3-α-glucosidase, glucan 1,4-α-maltotetraohydrolase, mycodextranase, glycosylceramidase, 1,2-α-L-fucosidase, 2,6-β-fructan 6-levanbiohydrolase, levanase, quercitrinase, galacturan 1,4-α-galacturonidase, isoamylase, glucan 1,6-α-glucosidase, glucan endo-1,2-β-glucosidase, xylan 1,3-β-xylosidase, licheninase, glucan 1,4-β-glucosidase, glucan endo-1,6-β-glucosidase, L-iduronidase, mannan 1,2-(1,3)-α-mannosidase, mannan endo-1,4-β-mannosidase, fructan β-fructosidase, β-agarase, exo-poly-α-galacturonosidase, κ-carrageenase, glucan 1,3-α-glucosidase, 6-phospho-3-galactosidase, 6-phospho-β-glucosidase, capsular-polysaccharide endo-1,3-α-galactosidase, non-reducing end β-L-arabinopyranosidase, arabinogalactan endo-β-1,4-galactanase, cellulose 1,4-O-cellobiosidase (non-reducing end), peptidoglycan β-N-acetylmuramidase, α,α-phosphotrehalase, glucan 1,6-α-isomaltosidase, dextran 1,6-α-isomaltotriosidase, mannosyl-glycoprotein endo-β-N-acetylglucosaminidase, endo-α-N-acetylgalactosaminidase, glucan 1,4-α-maltohexaosidase, arabinan endo-1,5-α-L-arabinanase, mannan 1,4-mannobiosidase, mannan endo-1,6-α-mannosidase, blood-group-substance endo-1,4-β-galactosidase, keratan-sulfate endo-1,4-β-galactosidase, steryl-O-glucosidase, strictosidine β-glucosidase, mannosyl-oligosaccharide glucosidase, protein-glucosylgalactosylhydroxylysine glucosidase, lactase, endogalactosaminidase, 1,3-α-L-fucosidase, 2-deoxyglucosidase, mannosyl-oligosaccharide 1,2-α-mannosidase, mannosyl-oligosaccharide 1,3-1,6-α-mannosidase, branched-dextran exo-1,2-α-glucosidase, glucan 1,4-α-maltotriohydrolase, amygdalin β-glucosidase, prunasin β-glucosidase, vicianin β-glucosidase, oligoxyloglucan β-glycosidase, polymannuronate hydrolase, maltose-6′-phosphate glucosidase, endoglycosylceramidase, 3-deoxy-2-octulosonidase, raucaffricine β-glucosidase, coniferin β-glucosidase, 1,6-α-L-fucosidase, glycyrrhizinate β-glucuronidase, endo-α-sialidase, glycoprotein endo-α-1,2-mannosidase, xylan α-1,2-glucuronosidase, chitosanase, glucan 1,4-α-maltohydrolase, difructose-anhydride synthase, neopullulanase, glucuronoarabinoxylan endo-1,4-β-xylanase, mannan exo-1,2-1,6-α-mannosidase, α-glucuronidase, lacto-N-biosidase, 4-α-D-{(1→4)-α-D-glucano}trehalose trehalohydrolase, limit dextrinase, poly(ADP-ribose) glycohydrolase, 3-deoxyoctulosonase, galactan 1,3-β-galactosidase, β-galactofuranosidase, thioglucosidase, β-primeverosidase, oligoxyloglucan reducing-end-specific cellobiohydrolase, xyloglucan-specific endo-β-1,4-glucanase, mannosylglycoprotein endo-β-mannosidase, fructan β-(2,1)-fructosidase, fructan β-(2,6)-fructosidase, xyloglucan-specific exo-β-1,4-glucanase, oligosaccharide reducing-end xylanase, ι-carrageenase, α-agarase, α-neoagaro-oligosaccharide hydrolase, β-apiosyl-β-glucosidase, λ-carrageenase, 1,6-α-D-mannosidase, galactan endo-1,6-β-galactosidase, exo-1,4-β-D-glucosaminidase, heparanase, baicalin-β-D-glucuronidase, hesperidin 6-O-α-L-rhamnosyl-β-D-glucosidase, protein O-GlcNAcase, mannosylglycerate hydrolase, rhamnogalacturonan hydrolase, unsaturated rhamnogalacturonyl hydrolase, rhamnogalacturonan galacturonohydrolase, rhamnogalacturonan rhamnohydrolase, β-D-glucopyranosyl abscisate β-glucosidase, cellulose 1,4-β-cellobiosidase (reducing end), α-D-xyloside xylohydrolase, β-porphyranase, gellan tetrasaccharide unsaturated glucuronyl hydrolase, unsaturated chondroitin disaccharide hydrolase, galactan endo-β-1,3-galactanase, 4-hydroxy-7-methoxy-3-oxo-3,4-dihydro-2H-1,4-benzoxazin-2-yl glucoside β-D-glucosidase, UDP-N-acetylglucosamine 2-epimerase (hydrolysing), UDP-N,N′-diacetylbacillosamine 2-epimerase (hydrolysing), non-reducing end β-L-arabinofuranosidase, protodioscin 26-O-β-D-glucosidase, (Ara-f)3-Hyp β-L-arabinobiosidase, avenacosidase, dioscin glycosidase (diosgenin-forming), dioscin glycosidase (3-O-β-D-Glc-diosgenin-forming), ginsenosidase type III, ginsenoside Rb1 β-glucosidase, ginsenosidase type I, ginsenosidase type IV, 20-O-multi-glycoside ginsenosidase, limit dextrin α-1,6-maltotetraose-hydrolase, β-1,2-mannosidase, α-mannan endo-1,2-α-mannanase, sulfoquinovosidase, exo-chitinase (non-reducing end), exo-chitinase (reducing end), endo-chitodextinase, carboxymethylcellulase, 1,3-α-isomaltosidase, isomaltose glucohydrolase, oleuropein β-glucosidase, and mannosyl-oligosaccharide α-1,3-glucosidase. In some embodiments the glycosyl hydrolase is selected from the group consisting of purine nucleosidase, inosine nucleosidase, uridine nucleosidase, AMP nucleosidase, NAD glycohydrolase, ADP-ribosyl cyclase/cyclic ADP-ribose hydrolase, adenosine nucleosidase, ribosylpyrimidine nucleosidase, adenosylhomocysteine nucleosidase, pyrimidine-5′-nucleotide nucleosidase, β-aspartyl-N-acetylglucosaminidase, inosinate nucleosidase, 1-methyladenosine nucleosidase, NMN nucleosidase, DNA-deoxyinosine glycosylase, methylthioadenosine nucleosidase, deoxyribodipyrimidine endonucleosidase, ADP-ribosylarginine hydrolase, DNA-3-methyladenine glycosylase I, DNA-3-methyladenine glycosylase II, rRNA N-glycosylase, DNA-formamidopyrimidine glycosylase, ADP-ribosyl-[dinitrogen reductase] hydrolase, N-methyl nucleosidase, futalosine hydrolase, uracil-DNA glycosylase, double-stranded uracil-DNA glycosylase, thymine-DNA glycosylase, aminodeoxyfutalosine nucleosidase, and adenine glycosylase.

In some embodiments, the enzyme portion of the fusion protein is O-GlcNAc transferase. In some embodiments, the enzyme portion comprises (i) a catalytic domain, and optionally, (ii) a tetratricopeptide repeat (TPR) domain. In some embodiments, the number of tetratricopeptide repeat (TPR) domains is selected from the group consisting of 0, 1, 2, 3, 4, 5, 6, 7, 8, 9, 10, 11, 12, and 13. In some embodiments, the number of TPR domains in the enzyme portion of the fusion protein is 0. In some embodiments, the number of TPR domains in the enzyme portion of the fusion protein is 4. In some embodiments, the number of TPR domains in the enzyme portion of the fusion protein is 13.

In some embodiments, the enzyme portion of the fusion protein is 0-GlcNAcase. In some embodiments, the enzyme portion of the fusion protein comprises (i) a catalytic domain, and optionally, (ii) a histone acetyltransferase (HAT)-like homology domain.

In some embodiments, the nanobody portion of the fusion protein selectively binds a target. In certain embodiments, the nanobody binds a cell surface protein. In certain embodiments, the nanobody binds a target selected from the group consisting of extracellular proteins, membrane proteins, nuclear proteins, cytosolic proteins, and mitochondrial proteins. In some embodiments, the nanobody binds a target selected from the group consisting of transcription factors, kinases, phosphatases, receptors, oxidoreductases, nucleoporins, and nuleosomes. In some embodiments, the nanobody binds a green fluorescent protein (GFP). In some embodiments, the nanobody binds TET3. In some embodiments, the nanobody binds Nup153. In certain embodiments, the nanobody binds H2B. In some embodiments, the nanobody binds Huntingtin. In certain embodiments, the nanobody binds alpha-synuclein. In some embodiments, the nanobody binds Tau. In certain embodiments, the nanobody binds a target selected from the group consisting of c-JUN, JUNB, IKZF1, STAT1. Zap70, Nup35, Nup62, H2B, H3, and H4.

In some embodiments, the nanobody binds a specific peptide tag or epitope. In some embodiments, the peptide tag is a 3, 4, 5, 6, 7, 8, 9, or 10 amino acid tag. In certain embodiments, the specific peptide tag is a four-amino acid tag. In some embodiments, the four-amino acid tag is EPEA. In some embodiments, the nanobody binds the four-amino acid EPEA tag (nEPEA). In certain embodiments, the epitope is selected from Myc-tag, HA-tag, FLAG-tag, GST-tag, 6×His, V5-tag, and OLLAS. In certain embodiments, the nanobody binds beta-catenin via recognition of a peptide tag.

### Linkers

In certain embodiments, the nanobody is fused to the glycan modifying enzyme via a linker. In certain embodiments, linkers may be used to link any of the proteins or protein domains described herein. The linker may be as simple as a covalent bond, or it may be a polymeric linker many atoms in length. In certain embodiments, the linker is a polypeptide or based on amino acids. In some embodiments, the linker is a short peptide sequence. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In certain embodiments, the linker comprises an aryl or heteroaryl moiety. In other embodiments, the linker comprises a polyethylene glycol moiety (PEG). In other embodiments, the linker comprises amino acids. In certain embodiments, the linker comprises a peptide.

The linker may include functionalized moieties to facilitate attachment of a nucleophile (e.g., thiol, amino) from the nanobody or enzyme to the linker. Any electrophile may be used as part of the linker. Exemplary electrophiles include, but are not limited to, activated esters, activated amides, Michael acceptors, alkyl halides, aryl halides, acyl halides, and isothiocyanates.

### Methods of Using Fusion Proteins

The present disclosure provides methods for adding or removing a glycan from a protein, and use thereof in treating or preventing diseases or disorders (e.g., neurodegenerative diseases (Parkinson's disease, Huntington's disease, Alzheimer's disease, dementia, multiple system atrophy), psychotic disorders (e.g., schizophrenia), epilepsy, sleep disorders, and addictions). Also provided herein, is the use of fusion proteins for diagnosing a subject with a disease.

In some embodiments, a glycan is added to or removed from a protein. In certain embodiments, the present disclosure provides methods of glycosylating a protein. In some embodiments, the present disclosure provides methods of removing a sugar from a protein.

In certain embodiments, the present disclosure provides methods of glycosylating a protein, the method comprising contacting a target protein with a fusion protein described herein. In certain embodiments, the stereochemistry of the donor molecule is retained. In certain embodiments, the stereochemistry of the donor molecule is inverted. In certain embodiments, the method involves the nucleophilic attack from the acceptor molecule. In certain embodiments, the method involves a dissociative reaction mechanism. In certain embodiments, the method involves a double displacement reaction mechanism. In certain embodiments, the method involves a single displacement reaction mechanism.

In some embodiments, the target protein is selected from the group consisting of nuclear proteins, cytosolic proteins, and mitochondrial proteins. In certain embodiments, the target protein is selected from the group consisting of transcription factors, kinases, phosphatases, oxidoreductases, nucleoporins, and nucleosomes.

In certain embodiments, the target protein is a transcription factor selected from the group consisting of AC008770.3, ACO23509.3, AC092835.1, AC138696.1, ADNP, ADNP2, AEBP1, AEBP2, AHCTF1, AHDC1, AHR, AHRR, AIRE, AKAP8, AKAP8L, AKNA, ALX1, ALX3, ALX4, ANHX, ANKZF1, AR, ARGFX, ARHGAP35, ARID2, ARID3A, ARID3B, ARID3C, ARID5A, ARID5B, ARNT, ARNT2, ARNTL, ARNTL2, ARX, ASCL1, ASCL2, ASCL3, ASCL4, ASCL5, ASH1L, ATF1, ATF2, ATF3, ATF4, ATF5, ATF6, ATF6B, ATF7, ATMIN, ATOH1, ATOH7, ATOH8, BACH1, BACH2, BARHL1, BARHL2, BARX1, BARX2, BATF, BATF2, BATF3, BAZ2A, BAZ2B, BBX, BCL11A, BCL11B, BCL6, BCL6B, BHLHA15, BHLHA9, BHLHE22, BHLHE23, BHLHE40, BHLHE41, BNC1, BNC2, BORCS-MEF2B, BPTF, BRF2, BSX, C11orf95, CAMTA1, CAMTA2, CARF, CASZ1, CBX2, CC2D1A, CCDC169-SOHLH2, CCDC17, CDC5, CDX1, CDX2, CDX4, CEBPA, CEBPB, CEBPD, CEBPE, CEBPG, CEBPZ, CENPA, CENPB, CENPBD1, CENPS, CENPT, CENPX, CGGBP1, CHAMP1, CHCHD3, CIC, CLOCK, CPEB1, CPXCR1, CREB1, CREB3, CREB3L1, CREB3L2, CREB3L3, CREB3L4, CREB5, CREBL2, CREBZF, CREM, CRX, CSRNP1, CSRNP2, CSRNP3, CTCF, CTCFL, CUX1, CUX2, CXXC1, CXXC4, CXXC5, DACH1, DACH2, DBP, DBX1, DBX2, DDIT3, DEAF1, DLX1, DLX2, DLX3, DLX4, DLX5, DLX6, DMBX1, DMRT1, DMRT2, DMRT3, DMRTA1, DMRTA2, DMRTB1, DMRTC2, DMTF1, DNMT1, DNTTIP1, DOT1L, DPF1, DPF3, DPRX, DR1, DRAP1, DRGX, DUX1, DUX3, DUX4, DUXA, DZIP1, E2F1, E2F2, E2F3, E2F4, E2F5, E2F6, E2F7, E2F8, E4F1, EBF1, EBF2, EBF3, EBF4, EEA1, EGR1, EGR2, EGR3, EGR4, EHF, ELF1, ELF2, ELF3, ELF4, ELF5, ELK1, ELK5, ELK4, EMX1, EMX2, EN1, EN2, EOMES, EPAS1, ERF, ERG, ESR1, ESR2, ESRRA, ESRRB, ESRRG, ESX1, ETS1, ETS2, ETV1, ETV2, ETV3, ETV3L, ETV4, ETV5, ETV6, ETV7, EVX1, EVX2, FAM170A, FAM200B, FBXL19, FERD3L, FEV, FEZF1, FEZF2, FIGLA, FIZ1, FIL1, FLYWCH1, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE5, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXS1, GABPA, GATA1, GATA2, GATA3, GATA4, GATA5, GATA6, GATAD2A, GATAD2B, GBX1, GBX2, GCM1, GCM2, GFI1, GFI1B, GLI1, GLI2, GLI3, GLI4, GLIS1, GLIS2, GLIS3, GLMP, GLYR1, GMEB1, GMEB2, GPBP1, GPBP1L1, GRHL1, GRHL2, GRHL3, GSC, GSC2, GSX1, GSX2, GTF2B, GTF2I, GTF2IRD1, GTF2IRD2, GTF2IRD2B, GTF3A, GZF1, HAND1, HAND2, HBP1, HDX, HELT, HES1, HES2, HES5, HES4, HES5, HES6, HEST, HESX1, HEY1, HEY2, HEYL, HHEX, HIC1, HIC2, HIF1A, HIF3A, HINFP, HIVEP1, HIVEP2, HIVEP3, HKR1, HLF, HLX, HMBOX1, HMG20A, HMG20B, HMGA1, HMGA2, HMGN3, HMX1, HMX2, HMX3, HNF1A, HNF1B, HNF4A, HNF4G, HOMEZ, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HSF1, HSF2, HSF4, HSF5, HSFX1, HSFX2, HSFY1, HSFY2, IKZF1, IKZF2, IKZF3, IKZF4, IKZF5, INSM1, INSM2, IRF1, IRF2, IRF3, IRF4, IRF5, IRF6, IRF7, IRF8, IRF9, IRX1, IRX2, IRX3, IRX4, IRX5, IRX6, ISL1, ISL2, ISX, JAZF1, JDP2, JRK, JRKL, JUN, JUNB, JUND, KAT7, KCMF1, KCNIP3, KDM2A, KDM2B, KDM5B, KIN, KLF1, KLF10, KLF11, KLF12, KLF13, KLF14, KLF15, KLF16, KLF17, KLF2, KLF3, KLF4, KLF5, KLF6, KLF7, KLF8, KLF9, KMT2A, KMT2B, L3MBTL1, L3MBTL3, L3MBTL4, LBX1, LBX2, LCOR, LCORL, LEF1, LEUTX, LHX1, LHX2, LHX3, LHX4, LHX5, LHX6, LHX8, LHX9, LIN28A, LIN28B, LIN54, LMX1A, LMX1B, LTF, LYL1, MAF, MAFA, MAFB, MAFF, MAFG, MAFK, MAX, MAZ, MBD1, MBD2, MBD3, MBD4, MBD6, MBNL2, MECOM, MECP2, MEF2A, MEF2B, MEF2C, MEF2D, MEIS1, MEIS2, MEIS3, MEOX1, MEOX2, MESP1, MESP2, MGA, MITF, MIXL1, MKX, MLX, MLXIP, MLXIPL, MNT, MNX1, MSANTD1, MSANTD3, MSANTD4, MSC, MSGN1, MSX1, MSX2, MTERF1, MTERF2, MTERF3, MTERF4, MTF1, MTF2, MXD1, MXD3, MXD4, MXI1, MYB, MYBL1, MYBL2, MYC, MYCL, MYCN, MYF5, MYF6, MYNN, MYOD1, MYOG, MYPOP, MYRF, MYRFL, MYSM1, MYT1, MYT1L, MZF1, NACC2, NAIF1, NANOG, NANOGNB, NANOGP8, NCOA1, NCOA2, NCOA3, NEUROD1, NEUROD2, NEUROD4, NEUROD6, NEUROG1, NEUROG2, NEUROG3, NFAT5, NFATC1, NFATC2, NFATC3, NFATC4, NFE2, NFE2L1, NFE2L2, NFE2L3, NFE4, NFIA, NFIB, NFIC, NFIL3, NFIX, NFKB1, NFKB2, NFX1, NFXL1, NFYA, NFYB, NFYC, NHLH1, NHLH2, NKRF, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NME2, NOBOX, NOTO, NPAS1, NPAS2, NPAS3, NPAS4, NROB1, NR1D1, NR1D2, NR1H2, NR1H3, NR1H4, NR1I2, NR1I3, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRF1, NRL, OLIG1, OLIG2, OLIG3, ONECUT1, ONECUT2, ONECUT3, OSR1, OSR2, OTP, OTX1, OTX2, OVOL1, OVOL2, OVOL3, PA2G4, PATZ1, PAX1, PAX2, PAX3, PAX4, PAX5, PAX6, PAX7, PAX8, PAX9, PBX1, PBX2, PBX3, PBX4, PCGF2, PCGF6, PDX1, PEG3, PGR, PHF1, PHF19, PHF20, PHF21A, PHOX2A, PHOX2B, PIN1, PITX1, PITX2, PITX3, PKNOX1, PKNOX2, PLAG1, PLAGL1, PLAGL2, PLSCR1, POGK, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PPARA, PPARD, PPARG, PRDM1, PRDM10, PRDM12, PRDM13, PRDM14, PRDM15, PRDM16, PRDM2, PRDM4, PRDM5, PRDM6, PRDM8, PRDM9, PREB, PRMT3, PROP1, PROX1, PROX2, PRR12, PRRX1, PRRX2, PTF1A, PURA, PURB, PURG, RAG1, RARA, RARB, RARG, RAX, RAX2, RBAK, RBCK1, RBPJ, RBPJL, RBSN, REL, RELA, RELB, REPIN1, REST, REXO4, RFX1, RFX2, RFX3, RFX4, RFX5, RFX6, RFX7, RFX8, RHOXF1, RHOXF2, RHOXF2B, RLF, RORA, RORB, RORC, RREB1, RUNX1, RUNX2, RUNX3, RXRA, RXRB, RXRG, SAFB, SAFB2, SALL1, SALL2, SALL3, SALL4, SATB1, SATB2, SCMH1, SCML4, SCRT1, SCRT2, SCX, SEBOX, SETBP1, SETDB1, SETDB2, SGSM2, SHOX, SHOX2, SIM1, SIM2, SIX1, SIX2, SIX3, SIX4, SIX5, SIX6, SKI, SKIL, SKOR1, SKOR2, SLC2A4RG, SMAD1, SMAD3, SMAD4, SMAD5, SMAD9, SMYD3, SNAI1, SNAI2, SNAI3, SNAPC2, SNAPC4, SNAPC5, SOHLH1, SOHLH2, SON, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPDEF, SPEN, SPI1, SPIB, SPIC, SPZ1, SRCAP, SREBF1, SREBF2, SRF, SRY, ST18, STAT1, STAT2, STAT5, STAT4, STAT5A, STA5B, STT6, T, TAL1, TAL2, TBP, TBPL1, TBPL2, TBR1, TBX1, TBX10, TBX15, TBX18, TBX19, TBX2, TBX20, TBX21, TBX22, TBX3, TBX4, TBX5, TBX6, TCF12, TCF15, TCF20, TCF21, TCF23, TCF24, TCF3, TCF4, TCF7, TCF7L1, TCF7L2, TCFL5, TEAD1, TEAD2, TEAD3, TEAD4, TEF, TERB1, TERF1, TERF2, TET1, TET2, TET3, TFAP2A, TFAP2B, TFAP2C, TFAP2D, TFAP2E, TFAP4, TFCP2, TFCP2L1, TFDP1, TFDP2, TFDP3, TFE3, TFEB, TFEC, TGIF1, TGIF2, TGIF2LX, TGIF2LY, THAP1, THAP10, THAP11, THAP12, THAP2, THAP3, THAP4, THAP5, THAP6, THAP7, THAP8, THAP9, THRA, THRB, THYN1, TIGD1, TIGD2, TIGD3, TIGD4, TIGD5, TIGD6, TIGD7, TLX1, TLX2, TLX3, TMF1, TOPORS, TP53, TP63, TP73, TPRX1, TRAFD1, TRERF1, TRPS1, TSC22D1, TSHZ1, TSHZ2, TSHZ3, TTF1, TWIST1, TWIST, UBP1, UNCX, USF1, USF2, USF3, VAX1, VAX2, VDR, VENTX, VEZF1, VSX1, VSX2, WIZ, WT1, XBP1, XPA, YBX1, YBX2, YBX3, YY1, YY2, ZBED1, ZBED2, ZBED3, ZBED4, ZBED5, ZBED6, ZBED9, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB9, ZC3H8, ZEB1, ZEB2, ZFAT, ZFHX2, ZFHX3, ZFHX4, ZFP1, ZFP14, ZFP2, ZFP28, ZFP3, ZFP30, ZFP37, ZFP41, ZFP42, ZFP57, ZFP62, ZFP64, ZFP69, ZFP69B, ZFP82, ZFP90, ZFP91, ZFP92, ZFPM1, ZFPM2, ZFX, ZFY, ZGLP1, ZGPAT, ZHX1, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT4, ZNF10, ZNF100, ZNF101, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, NF548, ZNF549, ZNF550, ZNF551, ZNF552, ZNF554, ZNF555, ZNF556, ZNF557, ZNF558, ZNF559, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF623, ZNF624, ZNF625, ZNF626, ZNF627, ZNF628, ZNF629, ZNF630, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF721, ZNF724, ZNF726, ZNF727, ZNF728, ZNF729, ZNF730, ZNF732, ZNF735, ZNF736, ZNF737, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF821, ZNF823, ZNF827, ZNF829, ZNF83, ZNF830, ZNF831, ZNF835, ZNF836, ZNF837, ZNF84, ZNF841, ZNF843, ZNF844, ZNF845, ZNF846, ZNF85, ZNF850, ZNF852, ZNF853, ZNF860, ZNF865, ZNF878, ZNF879, ZNF880, ZNF883, ZNF888, ZNF891, ZNF90, ZNF91, ZNF92, ZNF93, ZNF98, ZNF99, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZUFSP, ZXDA, ZXDB, ZXDC, ZZZ3.

In certain embodiments, the target protein is a kinase selected from the group consisting of AAK1, ABL, ACK, ACTR2, ACTR2B, AKT1, AKT2, AKT3, ALK, ALK1, ALK2, ALK4, ALK7, AMPKa1, AMPKa2, ANKRD3, ANPa, ANPb, ARAF, ARAFps, ARG, AurA, AurAps1, AurAps2, AurB, AurBps1, AurC, AXL, BARK1, BARK2, BIKE, BLK, BMPR1A, BMPR1Aps1, BMPR1Aps2, BMPR1B, BMPR2, BMX, BRAF, BRAFps, BRK, BRSK1, BRSK2, BTK, BUB1, BUBR1, CaMK1a, CaMK1b, CaMK1d, CaMK1g, CaMK2a, CaMK2b, CaMK2d, CaMK2g, CaMK4, CaMKK1, CaMKK2, caMLCK, CASK, CCK4, CCRK, CDC2, CDC7, CDK10, CDK11, CDK2, CDK3, CDK4, CDK4ps, CDK5, CDK5ps, CDK6, CDK7, CDK7ps, CDK8, CDK8ps, CDK9, CDKL1, CDKL2, CDKL3, CDKL4, CDKL5, CGDps, CHED, CHK1, CHK2, CHK2ps1, CHK2ps2, CK1a, CK1a2, CK1aps1, CK1aps2, CK1aps3, CK1d, CK1e, CK1g1, CK1g2, CK1g2ps, CK1g3, CK2a1, CK2a1-rs, CK2a2, CLIK1, CLIK1L, CLK1, CLK2, CLK2ps, CLK3, CLK3ps, CLK4, COT, CRIK, CRK7, CSK, CTK, CYGD, CYGF, DAPK1, DAPK2, DAPK3, DCAMKL1, DCAMKL2, DCAMKL3, DDR1, DDR2, DLK, DMPK1, DMPK2, DRAK1, DRAK2, DYRK1A, DYRK1B, DYRK2, DYRK3, DYRK4, EGFR, EphA1, EphA10, EphA2, EphA3, EphA4, EphA5, EphA6, EphA7, EphA8, EphB1, EphB2, EphB3, EphB4, EphB6, Erk1, Erk2, Erk3, Erk3ps1, Erk3ps2, Erk3ps3, Erk3ps4, Erk4, Erk5, Erk7, FAK, FER, FERps, FES, FGFR1, FGFR2, FGFR3, FGFR4, FGR, FLT1, FLT1ps, FLT3, FLT4, FMS, FRK, Fused, FYN, GAK, GCK, GCN2, GCN22, GPRK4, GPRK5, GPRK6, GPRK6ps, GPRK7, GSK3A, GSK3B, Haspin, HCK, HER2/ErbB2, HER3/ErbB3, HER4/ErbB4, HH498, HIPK1, HIPK2, HIPK3, HIPK4, HPK1, HRI, HRIps, HSER, HUNK, ICK, IGF1R, IKKa, IKKb, IKKe, ILK, INSR, IRAK1, IRAK2, IRAK3, IRAK4, IRE1, IRE2, IRR, ITK, JAK1, JAK2, JAK3, JNK1, JNK2, JNK3, KDR, KHS1, KHS2, KIS, KIT, KSGCps, KSR1, KSR2, LATS1, LATS2, LCK, LIMK1, LIMK2, LIMK2ps, LKB1, LMR1, LMR2, LMR3, LOK, LRRK1, LRRK2, LTK, LYN, LZK, MAK, MAP2K1, MAP2K1ps, MAP2K2, MAP2K2ps, MAP2K3, MAP2K4, MAP2K5, MAP2K6, MAP2K7, MAP3K1, MAP3K2, MAP3K3, MAP3K4, MAP3K5, MAP3K6, MAP3K7, MAP3K8, MAPKAPK2, MAPKAPK3, MAPKAPK5, MAPKAPKps1, MARK1, MARK2, MARK5, MARK4, MARKps01, MARKps02, MARKps03, MARKps04, MARKps05, MARKps07, MARKps08, MARKps09, MARKps10, MARKps11, MARKps12, MARKps13, MARKps15, MARKps16, MARKps17, MARKps18, MARKps19, MARKps20, MARKps21, MARKps22, MARKps23, MARKps24, MARKps25, MARKps26, MARKps27, MARKps28, MARKps29, MARKps30, MAST1, MAST2, MAST5, MAST4, MASTL, MELK, MER, MET, MISR2, MLK1, MLK2, MLK3, MLK4, MLKL, MNK1, MNK1ps, MNK2, MOK, MOS, MPSK1, MPSK1ps, MRCKa, MRCKb, MRCKps, MSK1, MSK12, MSK2, MSK22, MSSK1, MST1, MST2, MST3, MST3ps, MST4, MUSK, MYO3A, MYO3B, MYT1, NDR1, NDR2, NEK1, NEK10, NEK11, NEK2, NEK2ps1, NEK2ps2, NEK2ps3, NEK3, NEK4, NEK4ps, NEK5, NEK6, NEK7, NEK8, NEK9, NIK, NIM1, NLK, NRBP1, NRBP2, NuaK1, NuaK2, Obscn, Obscn2, OSR1, p38a, p38b, p38d, p38g, p70S6K, p70S6Kb, p70S6Kps1, p70S6Kps2, PAK1, PAK2, PAK2ps, PAK3, PAK4, PAK5, PAK6, PASK, PBK, PCTAIRE1, PCTAIRE2, PCTAIRE3, PDGFRa, PDGFRb, PDK1, PEK, PFTAIRE1, PFTAIRE2, PHKg1, PHKg1ps1, PHKg1ps2, PHKg1ps3, PHKg2, PIK3R4, PIM1, PIM2, PIM3, PINK1, PITSLRE, PKACa, PKACb, PKACg, PKCa, PKCb, PKCd, PKCe, PKCg, PKCh, PKCi, PKCips, PKCt, PKCz, PKD1, PKD2, PKD3, PKG1, PKG2, PKN1, PKN2, PKN3, PKR, PLK1, PLK1ps1, PLK1ps2, PLK2, PLK3, PLK4, PRKX, PRKXps, PRKY, PRP4, PRP4ps, PRPK, PSKH1, PSKH1ps, PSKH2, PYK2, QIK, QSK, RAF1, RAF1ps, RET, RHOK, RIPK1, RIPK2, RIPK3, RNAseL, ROCK1, ROCK2, RON, ROR1, ROR2, ROS, RSK1, RSK12, RSK2, RSK22, RSK3, RSK32, RSK4, RSK42, RSKL1, RSKL2, RYK, RYKps, SAKps, SBK, SCYL1, SCYL2, SCYL2ps, SCYL3, SGK, SgK050ps, SgK069, SgK071, SgK085, SgK110, SgK196, SGK2, SgK223, SgK269, SgK288, SGK3, SgK307, SgK384ps, SgK396, SgK424, SgK493, SgK494, SgK495, SgK496, SIK (e.g., SIK1, SIK2), skMLCK, SLK, Slob, smMLCK, SNRK, SPEG, SPEG2, SRC, SRM, SRPK1, SRPK2, SRPK2ps, SSTK, STK33, STK33ps, STLK3, STLK5, STLK6, STLK6ps1, STLK6-rs, SuRTK106, SYK, TAK1, TAO1, TAO2, TAO3, TBCK, TBK1, TEC, TESK1, TESK2, TGFbR1, TGFbR2, TIE1, TIE2, TLK1, TLK1ps, TLK2, TLK2ps1, TLK2ps2, TNK1, Trad, Trb1, Trb2, Trb3, Trio, TRKA, TRKB, TRKC, TSSK1, TSSK2, TSSK3, TSSK4, TSSKps1, TSSKps2, TTBK1, TTBK2, TTK, TTN, TXK, TYK2, TYK22, TYRO3, TYRO3ps, ULK1, ULK2, ULK3, ULK4, VACAMKL, VRK1, VRK2, VRK3, VRK3ps, Wee1, Wee1B, Wee1Bps, Wee1ps1, Wee1ps2, Wnk1, Wnk2, Wnk3, Wnk4, YANK1, YANK2, YANK5, YES, YESps, YSK1, ZAK, ZAP70, ZC1/HGK, ZC2/TNIK, ZC3/MINK, and ZC4/NRK.

In some embodiments, the transcription factor is selected from the group consisting of c-JUN, JUNB, IKZF1, and STAT1. In certain embodiments, the kinase is Zap70. In some embodiments, the oxidoreductase is TET3. In some embodiments, the nucleoporin is selected from the group consisting of Nup35 and Nup62. In some embodiments, the nucleosome is selected from the group consisting of H2B, H3, and H4.

In certain embodiments, the target protein is alpha-synuclein. In some embodiments, the target protein is Tau. In certain embodiments, the target protein is Huntingtin.

In certain embodiments, the present disclosure provides methods of glycosylating a protein, the method comprising contacting a target protein with a fusion protein in the presence of a glycosyl donor molecule, thereby installing the sugar moiety from the glycosyl donor molecule on the target protein. In some embodiments, the present disclosure provides methods of glycosylating a protein, the method comprising contacting a target protein with a fusion protein in the presence of a O-linked N-acetyl glucosamine donor molecule, thereby installing a O-linked N-acetyl glucosamine on the target protein via the addition of a glucosamine monosaccharide attached to serine or threonine. In certain embodiments the monosaccharide is serine. In some embodiments, the monosaccharide is threonine. In certain embodiments, the glycosyl donor molecule is selected from the group consisting of uridine diphospho-D-glucose, uridine diphospho-D-galactose, uridine diphospho-D-xylose, uridine diphospho-N-acetyl-D-glucosamine, uridine diphospho-N-acetyl-D-galactosamine, uridine diphospho-D-glucuronic acid, uridine diphospho-D-galactofuranose, guanosine diphospho-D-mannose, guanosine diphospho-L-fucose, guanosine diphospho-L-rhamnose, cytidine monophospho-N-acetylneuraminic acid, and cytidine monophospho-2-keto-3-deoxy-D-mannooctanoic acid. In certain embodiments, the glycosyl donor molecule is selected from the group consisting of N-azidoacetylglucosamine (GlcNAz), N-azidoactylgalactosamine (GalNAz), N-azidoacetylfucosamine (FucNAz), and FucAl. In some embodiments, the target protein is alpha-synuclein. In some embodiments, the target protein is Tau. In certain embodiments, the target protein is Huntingtin. In certain embodiments, the target protein is beta-catenin. Exemplary target proteins include c-JUN, JUNB, IKZF1, STAT1, Zap70, TET3, Nup35, Nup62, H2B, H3, H4, beta-catenin, alpha-synuclein, Huntingtin, and Tau.

In some embodiments, the present disclosure provides methods of removing a sugar from a protein. In some embodiments, the method of removing a sugar from a protein comprises contacting a target protein containing a sugar with a fusion protein, thereby excising a sugar moiety from the target protein. In some embodiments, the method of removing a sugar from a protein comprises contacting a protein containing an O-linked N-acetyl glucosamine with a fusion protein described herein, thereby excising an O-linked N-acetyl glucosamine. In certain embodiments, O-linked N-acetyl glucosamine is removed from a serine or threonine residue of the protein. Exemplary target proteins include c-JUN, JUNB, IKZF1, STAT1, Zap70, TET3, Nup35, Nup62, H2B, H3, H4, beta-catenin, alpha-synuclein, Huntingtin, and Tau. In certain embodiments, the target protein is alpha-synuclein. In some embodiments, the target protein is Tau. In certain embodiments, the target protein is Huntingtin.

Further provided in the present disclosure are methods of treating and diagnosing diseases. Further provided in the present disclosure are methods of treating diseases. In some embodiments, the present disclosure provides methods of treating a disease, the method comprising administering a fusion protein to a subject in need thereof. Further provided in the present disclosure are methods of diagnosing diseases. In some embodiments, the present disclosure provides methods of diagnosing a subject with a disease, the method comprising administering a fusion protein described herein to a subject. In certain embodiments, the diagnosing occurs in an ex-vivo sample taken from a subject wherein glycosylation on specific target proteins is monitored.

In some embodiments, the present disclosure provides methods of treating a subject suffering from or susceptible to a neurodegenerative disease, the method comprising administering an effective amount of the fusion protein. In certain embodiments, the neurodegenerative disease is selected from the group consisting of Parkinson's disease, Huntington's disease, Alzheimer's disease, dementia, and multiple system atrophy. In some embodiments, the neurodegenerative disease is Parkinson's disease. In some embodiments, the neurodegenerative disease is Huntington's disease.

In some embodiments, the present disclosure provides methods of treating a subject suffering from or susceptible to a psychotic disorder, the method comprising administering an effective amount of the fusion protein. In certain embodiments, the psychotic disorder is schizophrenia.

In some embodiments, the present disclosure provides methods of treating a subject suffering from or susceptible to epilepsy, the method comprising administering an effective amount of the fusion protein. In some embodiments, the present disclosure provides methods of treating a subject suffering from or susceptible to a sleep disorder, the method comprising administering an effective amount of the fusion protein. In certain embodiments, the present disclosure provides methods of treating a subject suffering from or susceptible to an addiction, the method comprising administering an effective amount of the fusion protein.

In some embodiments, the subject suffering from or susceptible to a disease is treated by administering a fusion protein which adds a glycan to the target protein, thereby altering the folding of the target protein. In some embodiments, the subject suffering from or susceptible to a disease is treated by administering a fusion protein which excises a glycan to the target protein, thereby altering the folding of the target protein.

In some embodiments, the subject suffering from or susceptible to a disease is treated by administering a fusion protein which adds a glycan to the target protein, thereby decreasing the tendency of the target protein to mis-fold. In some embodiments, the subject suffering from or susceptible to a disease is treated by administering a fusion protein which excises a glycan to the target protein, thereby decreasing the tendency of the target protein to mis-fold. In some embodiments, the subject suffering from or susceptible to a disease is treated by administering a fusion protein which adds a glycan to the target protein, thereby altering the folding of the target protein resulting in a conformational change decreasing the tendency of the target protein to bind to itself. In some embodiments, the subject suffering from or susceptible to a disease is treated by administering a fusion protein which excises a glycan to the target protein, thereby altering the folding of the target protein resulting in a conformational change decreasing the tendency of the target protein to bind to itself.

In some embodiments, the subject suffering from or susceptible to a disease is treated by administering a fusion protein which adds a glycan to the target protein, thereby altering the mis-folding of the target protein. In some embodiments, the subject suffering from or susceptible to a disease is treated by administering a fusion protein which adds a glycan to alpha-synuclein, thereby altering the mis-folding of the target protein.

In some embodiments, the subject suffering from or susceptible to a disease is treated by administering a fusion protein which adds a glycan to the target protein, thereby altering the ability of the target protein to form protein aggregates. In some embodiments, the subject suffering from or susceptible to a disease is treated by administering a fusion protein which adds a glycan to alpha-synuclein, thereby altering the ability of the target protein to form protein aggregates. In some embodiments, the subject suffering from or susceptible to a disease is treated by administering a fusion protein which adds a glycan to tau, thereby altering the ability of the target protein to form protein aggregates. In some embodiments, the subject suffering from or susceptible to a disease is treated by administering a fusion protein which adds a glycan to Huntingtin, thereby altering the ability of the target protein to form protein aggregates.

In some embodiments, the subject suffering from or susceptible to a disease is treated by administering a fusion protein which excises a glycan from the target protein, thereby altering the ability of the target protein to form protein aggregates. In some embodiments, the subject suffering from or susceptible to a disease is treated by administering a fusion protein which excises a glycan from alpha-synuclein, thereby altering the ability of the target protein to form protein aggregates. In some embodiments, the subject suffering from or susceptible to a disease is treated by administering a fusion protein which excises a glycan from tau, thereby altering the ability of the target protein to form protein aggregates. In some embodiments, the subject suffering from or susceptible to a disease is treated by administering a fusion protein which adds a glycan to Huntingtin, thereby altering the ability of the target protein to form protein aggregates.

In some embodiments, the subject suffering from or susceptible to a disease is treated by administering a fusion protein which adds a glycan to the target protein, thereby altering the protein aggregate involving the target protein. In some embodiments, the subject suffering from or susceptible to a disease is treated by administering a fusion protein which adds a glycan to alpha-synuclein, thereby altering the protein aggregate involving the target protein. In some embodiments, the subject suffering from or susceptible to a disease is treated by administering a fusion protein which adds a glycan to tau, thereby altering the protein aggregate involving the target protein. In some embodiments, the subject suffering from or susceptible to a disease is treated by administering a fusion protein which adds a glycan to Huntingtin, thereby altering the protein aggregate involving the target protein.

In some embodiments, the subject suffering from or susceptible to a disease is treated by administering a fusion protein which excises a glycan from the target protein, thereby altering the protein aggregate involving the target protein. In some embodiments, the subject suffering from or susceptible to a disease is treated by administering a fusion protein which excises a glycan from alpha-synuclein, thereby altering the protein aggregate involving the target protein. In some embodiments, the subject suffering from or susceptible to a disease is treated by administering a fusion protein which excises a glycan from tau, thereby altering the protein aggregate involving the target protein. In some embodiments, the subject suffering from or susceptible to a disease is treated by administering a fusion protein which adds a glycan to Huntingtin, thereby altering the protein aggregate involving the target protein.

### Kits

In some embodiments, the present disclosure provides kits. In certain embodiments, the kit comprises a fusion protein described and a glycosyl donor molecule. In some embodiments, the kit comprises a fusion protein and uridine diphosphate N-acteylglucosamine. In some embodiments, the kit comprises a vector for expressing a fusion protein and a glycosyl acceptor molecule. In some embodiments, the kit comprises a vector for expressing a fusion protein and a glycosyl donor molecule. In some embodiments, the kit comprises a vector for expressing a fusion protein and uridine diphosphate N-acteylglucosamine. In some embodiments, the glycosyl donor molecule is selected from the group consisting of uridine diphospho-D-glucose, uridine diphospho-D-galactose, uridine diphospho-D-xylose, uridine diphospho-N-acetyl-D-glucosamine, uridine diphospho-N-acetyl-D-galactosamine, uridine diphospho-D-glucuronic acid, uridine diphospho-D-galactofuranose, guanosine diphospho-D-mannose, guanosine diphospho-L-fucose, guanosine diphospho-L-rhamnose, cytidine monophospho-N-acetylneuraminic acid, and cytidine monophospho-2-keto-3-deoxy-D-mannooctanoic acid.

The kits described herein may include one or more containers housing components for performing the methods described herein and optionally instructions for uses. Any of the kit described herein may further comprise components needed for performing the methods. Each component of the kits, where applicable, may be provided in liquid form (e.g., in solution), or in solid form, (e.g., a dry powder). In certain cases, some of the components may be reconstitutable or otherwise processible (e.g., to an active form), for example, by the addition of a suitable solvent or other species (e.g., water or buffer), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. As used herein, “promoted” includes all methods of doing business including methods of education, scientific inquiry, academic research, and any advertising or other promotional activity including written, oral and electronic communication of any form, associated with the disclosure. Additionally, the kits may include other components depending on the specific application, as described herein.

The kits may contain any one or more of the components described herein in one or more containers. The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may also include other components, depending on the specific application, for example, containers, cell media, salts, buffers, reagents, etc.

### ADDITIONAL EMBODIMENTS

In some embodiments, the present disclosure provides a polynucleotide encoding a fusion protein. In some embodiments, the present disclosure provides vector comprising the polynucleotide encoding a fusion protein described herein.

In some embodiments, the present disclosure provides a cell comprising a fusion protein. In some embodiments, the present disclosure provides a cell comprising the nucleic acid molecule encoding a fusion protein.

### EXAMPLES

In order that the present disclosure may be more fully understood, the following examples are set forth. The examples described in this application are offered to illustrate the fusion proteins, compositions, kits, uses, and methods provided herein and are not to be construed in any way as limiting their scope.

### General Information, Methods, and Analysis Techniques

At least some of the reactions were performed in single-neck, oven-dried, round-bottomed flasks fitted with rubber septa under a positive pressure of nitrogen. Organic solutions were concentrated by rotary evaporation at 30-35° C. Normal-phase purifications were performed using silica gel (60 Å, 40-63 μm particle size) purchased from Silicycle (Quebec, Canada). Analytical thin-layer chromatography (TLC) was performed using glass plates pre-coated with silica gel (0.25 mm, 60 Å pore size) impregnated with a fluorescent indicator (254 nm). TLC plates were visualized by exposure to ultraviolet light (UV), and/or submersion in KMnO₄ or ninhydrin solution followed by brief heating with a heat gun (10-15 s). Commercial chemical materials, solvents, and reagents were used as received with the following exceptions. Triethylamine was distilled from calcium hydride under an atmosphere of nitrogen before use.

Ac₄GalNAz was synthesized according to the procedure of Bertozzi and co-workers (Hang, H. C.; Yu, C.; Kato, D. L.; Bertozzi, C. R. Proceedings of the National Academy of Sciences 2003, 100, 14846) and dissolved in DMSO to obtain a 10 mM stock solution. For long-term storage, the stock solution was stored in amber microcentrifuge tubes at −80° C.

The cleavable biotin silane probe as a 1:3 ratio mixture of the light and heavy (+2 deuteriums) stable isotopes was prepared according to the procedure of Bertozzi and co-workers (Woo, C. M.; Felix, A.; Byrd, W. E.; Zuegel, D. K.; Ishihara, M.; Azadi, P.; Iavarone, A. T.; Pitteri, S. J.; Bertozzi, C. R. Journal of Proteome Research 2017, 16, 1706). The cleavable biotin silane probe was dissolved in DMSO to obtain a 10 mM stock solution and kept in amber microcentrifuge tubes at −20° C. for short-term storage and kept as in solid form at −80° C. for long-term storage.

RapiGest was prepared according to the method of Lee and co-workers (Lee, P. J. J.; Compton, B. J., U.S. Pat. No. 7,229,539, issued Jun. 12, 2007). RapiGest was stored as a solid at −20° C. and dissolved in PBS as needed.

Peracetylated 5S-GlcNAc was synthesized according to the reported procedure Vocadlo and co-workers (Gloster, T. M.; Zandberg, W. F.; Heinonen, J. E.; Shen, D. L.; Deng, L.; Vocadlo, D. J., Nature Chemical Biology, 2011, 7, 174). Peracetylated 5S-GlcNAc was dissolved in DMSO to obtain a 100 mM stock solution and stored in amber microcentrifuge tubes at −80° C. for long-term storage.

All antibodies were diluted in 3% BSA/TBST, unless otherwise noted.

TABLE I
Antibodies

| No. | Antibody name | Host species | Dilution | Commercial source |
|---|---|---|---|---|
| 1 | Flag (M2) | Mouse mAb | 1:4,000 | Sigma-Aldrich |
| 2 | HA-Tag (C29F4) | Rabbit mAb | 1:1,000 | Cell Signaling |
| 3 | O-GlcNAc | Mouse mAb | 1:1,000 | Cell Signaling |
| | (CTD110.6) | | | |
| 4 | OGT (D1D8Q) | Rabbit mAb | 1:1,000 | Cell Signaling |
| 5 | GFP (B-2) | Mouse | 1:1,000 | Santa Cruz Biotechnology |
| 6 | CREB (86B10) | Mouse | 1:1,000 | Cell signaling |
| 7 | Streptavidin-HRP | | 1:10,000 | Thermo Fisher |
| 8 | Alexa Fluor 594 | Goat | 1:5,000 | Thermo Fisher/Invitrogen |
| 9 | Alexa Fluor 488 | Goat | 1:5,000 | Thermo Fisher/Invitrogen |
| 10 | Anti-mouse-HRP | Goat | 1:10,000 | Rockland |
| | | | | Immunochemicals |
| 11 | Anti-rabbit-HRP | Goat | 1:10,000 | Rockland |
| | | | | Immunochemicals |
| 12 | Anti-mouse-IR800 | Goat | 1:10,000 | Azure Biosystems |
| 13 | Anti-rabbit-IR700 | Goat | 1:10,000 | Azure Biosystems |

Molecular cloning reagents were purchased from New England Biolabs.

TABLE 2
Molecular Cloning Reagents

| No. | Antibody name |
|---|---|
| 1 | Gibson Assembly Master mix |
| 2 | Q5 High-Fidelity 2X Master Mix |
| 3 | T4 Polynucleotide Kinase |
| 4 | HindIII-HF |
| 5 | NotI-HF |
| 6 | Sgsl |
| 7 | Sgfl |
| 8 | BamHI-HF |
| 9 | XhoI |

TABLE 3
Addgene Plasmids

| No. | Plasmid name | Source or Addgene Plasmid # |
|---|---|---|
| 1 | pEXP pLHCX ncOGT | ACS Chem. Biol. 2017, 12, 787 |
| 2 | pCSDEST2-APEX2-GBP | 67651 |
| 3 | pCS-H2B-mRFP | 53745 |
| 4 | mOrange2-H3-23 | 57963 |
| 5 | mPlum-H4-23 | 55979 |
| 6 | pEGFP-C1-Nup35 | 87342 |
| 7 | pEGFP-(C3)-Nup153 | 64268 |
| 8 | pDONR223-NUP62 | 23559 |
| 9 | FH-TET3-pEF | 49446 |
| 10 | cJun | Mol. Cell. Proteomics 2018, 17, 764 |
| 11 | STAT1 | Mol. Cell. Proteomics 2018, 17, 764 |
| 12 | JunB | Mol. Cell. Proteomics 2018, 17, 764 |
| 13 | pPHAGE-IKZF1 | Harvard Plasmid Repository; |
| | | cat #HsCD00456010 |

All plasmids were derived from the Invitrogen pcDNA3.1 vector, which contains a CMV promoter for constitutive expression.

TABLE 4
Plasmids

| No. | Plasmid No. | Plasmid name |
|---|---|---|
| 1 | pWLH085 | pcDNA3.1-HA-nGFP-(EAAAK) |
| | | 4-OGT (1-1046) |
| 2 | pWLH189 | pcDNA3.1-HA-nGFP-(EAAAK) |
| | | 4-OGT (327-1046) |
| 3 | pWLH015 | pcDNA3.1-HA-nGFP-(EAAAK) |
| | | 4-OGT (463-1046) |
| 4 | pWLH086 | pcDNA3.1-HA-nEPEA-(EAAAK) |
| | | 4-OGT (1-1046) |
| 5 | pWLH137 | pcDNA3.1-HA-nEPEA-(EAAAK) |
| | | 4-OGT (327-1046) |
| 6 | pWLH138 | pcDNA3.1-HA-nEPEA-(EAAAK) |
| | | 4-OGT (463-1046) |
| 7 | pWLH118 | pcDNA3.1-HA-OGT (1-1046) |
| 8 | pWLH117 | pcDNA3.1-HA-OGT (327-1046) |
| 9 | pWLH119 | pcDNA3.1-HA-OGT (463-1046) |
| 10 | pWLH147 | pcDNA3.1-Nup62-Flag-EPEA |
| 11 | pWLH133 | pcDNA3.1-Nup35-Flag-EPEA |
| 12 | pWLH142 | pcDNA3.1-cJun-Flag-EPEA |
| 13 | pWLH082 | pcDNA3.1-JunB-Flag-EPEA |
| 14 | pWLH145 | pcDNA3.1-Zap70-Flag-EPEA |
| 15 | pWLH083 | pcDNA3.1-IKZF1-Flag-EPEA |
| 16 | pWLH084 | pcDNA3.1-STAT1-Flag-EPEA |
| 17 | pWLH161 | pcDNA3.1-H2B-Flag-EPEA |
| 18 | pWLH162 | pcDNA3.1-H3-Flag-EPEA |
| 19 | pWLH163 | pcDNA3.1-H4-Flag-EPEA |
| 20 | pWLH134 | pcDNA3.1-TET3 (1-1660)- |
| | | Flag-EPEA (Human) |
| 21 | pWLH191 | pcDNA3.1-TET3 (680-1660)- |
| | | Flag-EPEA (Human) |
| 22 | pWLH113 | pcDNA3.1-GFP-Flag-cJun-EPEA |

TABLE 5
Primers

| No | Primer name | Sequence (5′ to 3′) |
|---|---|---|
| 1 | HA-nGFP- | CCCAAGCTGGCGAGCGTT |
| | (EAAAK)4-OGT(1- | TAAGCTTGAGCAATGGCA |
| | 1046) fwd nGFP | TACCCATACGATGTTCCA |
| | | GATTACGCTGCGATCGCA |
| | | CAGGTGCAGCTGGTGGAG |
| | | TCTGGAGGA |
| | | (SEQ ID NO: 1) |
| 2 | HA-nGFP- | GGATCCCTTTGCAGCTGC |
| | (EAAAK)4-OGT(1- | CTCCTTTGCAGCTGCCTC |
| | 1046) rev nGFP | CTTTGCAGCTGCCTCCTT |
| | | TGCAGCTGCCTCTGGCGC |
| | | GCCAGAGCTCACTGTCAC |
| | | CTGTGTT |
| | | (SEQ ID NO: 2) |
| 3 | HA-nGFP- | AAAGGAGGCAGCTGCAAA |
| | (EAAAK)4-OGT(1- | GGAGGCAGCTGCAAAGGG |
| | 1046) fwd OGT | ATCCATGGCGTCTTCCGT |
| | | GGGCAA |
| | | (SEQ ID NO: 3) |
| 4 | HA-nGFP- | CGGGTTTAAACGGGCCCT |
| | (EAAAK)4-OGT(1- | CTAGACTCGAGCGGCCGC |
| | 1046) rev OGT | TTAGGCTGACTCGGTGAC |
| | | TTCAACAGGCTTAATCAT |
| | | GTGGTC |
| | | (SEQ ID NO: 4) |
| 5 | HA-nEPEA-X-X | TACGCTGCGATCGCAATG |
| | fwd | GGCCAGCTGGTGGAGA |
| | | (SEQ ID NO: 5) |
| 6 | HA-nEPEA-X-X | CTGGCGCGCCAGAGCTCA |
| | rev | CAGTAACCTGGGTGCC |
| | | (SEQ ID NO: 6) |
| 7 | NtermOGT(327- | AAAGGGATCCATGGCAGA |
| | 1046)BamHI | CTCTTTGAATAACCTTGC |
| | fwd | CAACATCAAACGGG |
| | | (SEQ ID NO: 7) |
| 8 | NtermOGT(463- | GCAAAGGGATCCCCTGAT |
| | 1046)BamHI | GCTTATTGTAACTTGGCT |
| | fwd | CATTGCC |
| | | (SEQ ID NO: 8) |
| 9 | pcDNA3-HA- | TTACGCTGCGATCGCAAT |
| | OGT( 1-1046) | GGCGTCTTCCGTGGGCAA |
| | fwd | CGTGGC |
| | | (SEQ ID NO: 9) |
| 10 | pcDNA3-HA- | TATAGCGGCCGCTGGCGC |
| | OGT( 1-1046) | GCCTTAGGCTGACTCGGT |
| | rev | GACTTCAACAGGCTTAAT |
| | | CATGTGGTCAGGTTTGTT |
| | | (SEQ ID NO: 10) |
| 11 | pcDNA3-HA- | ACGCTGCGATCGCAATGG |
| | OGT(327-1046) | CAGACTCTTTGAATAACC |
| | fwd | TTGCCAACATCAAACGGG |
| | | AACAGGGC |
| | | (SEQ ID NO: 11) |
| 12 | pcDNA3-HA- | CGCTGCGATCGCAATGCC |
| | OGT(463-1046) | TGATGCTTATTGTAACTT |
| | fwd | GGCTCATTGCCTACAGAT |
| | | TGTCTGTGATTGGACAGA |
| | | CTATGATGAGCGG |
| | | (SEQ ID NO: 12) |
| 13 | Nup62-Flag- | ACTTAAGCTTGGGCGATC |
| | EPEA fwd | GCAATGGCAAGCGGGTTT |
| | | AATTTTGG |
| | | (SEQ ID NO: 13) |
| 14 | Nup62-Flag- | CTCTAGACTCGAGTTATG |
| | EPEA rev | CTTCAGGTTCCTTATCGT |
| | | CGTCATCCTTGTAGTCTG |
| | | (SEQ ID NO: 14) |
| 15 | Sgfl-H2B-Sgsl | CAGGCGATCGCAATGGCA |
| | fwd | CCAGAGCCAGCGAAGTCT |
| | | (SEQ ID NO: 15) |
| 16 | Sgfl-H2B-Sgsl | GTCTGGCGCGCCCTTAGCG |
| | rev | CTGGTGTACTTGGTG |
| | | (SEQ ID NO: 16) |
| 17 | Sgfl-H3-Sgsl | AGCAGGCGATCGCAATGGC |
| | fwd | TCGTACTAAACAGACAGC |
| | | TCGG |
| | | (SEQ ID NO: 17) |
| 18 | Sgfl-H3-Sgsl | TCTGGCGCGCCCGCTCTTT |
| | rev | CTCCGCGAAT |
| | | (SEQ ID NO: 18) |
| 19 | Sgfl-H4-Sgsl | AGCAGGCGATCGCAATGTC |
| | fwd | TGGCCGCGGCAAAGG |
| | | (SEQ ID NO: 19) |
| 20 | Sgfl-Nup35-Sgsl | AGGCGATCGCAATGGCAGC |
| | fwd | CTTTGCAGTGGAACC |
| | | (SEQ ID NO: 20) |
| 21 | Sgfl-Nup35-Sgsl | TCTGGCGCGCCCCAGCCAA |
| | rev | ACATGTACTCCATTGC |
| | | (SEQ ID NO: 21) |
| 22 | Sgfl-TET3-Sgsl | AGCAGGCGATCGCAATGGA |
| | fwd | CTCAGGGCCAGTGTACC |
| | | (SEQ ID NO: 22) |
| 23 | Sgfl-TET3-Sgsl | TCTGGCGCGCCGATCCAGC |
| | rev | GGCTGTAGGG |
| | | (SEQ ID NO: 23) |
| 24 | Sgfl-TET3(680- | GACACACCTGCCAAGAGAG |
| | 1660)-Sgsl | CCCAGGCCGAGTTC |
| | fwd | (SEQ ID NO: 24) |
| 25 | Sgfl-TET3(680- | CATTGCGATCGCCCAAGCT |
| | 1660)-Sgsl | TAAGTTTAAACGCTAGCCA |
| | rev | GCTTGGGTCTCC |
| | | (SEQ ID NO: 25) |
| 26 | Sgfl-cJun-Sgsl | CTGGCAGGCGATCGCAATG |
| | fwd | ACTGCAAAGATGGAAACG |
| | | ACC(SEQ ID NO: 26) |
| 27 | Sgfl-cJun-Sgsl | TCTGGCGCGCCAAATGTTT |
| | rev | GCAACTGCTGCGTTAGC |
| | | (SEQ ID NO: 27) |
| 28 | Sgfl-JunB-Sgsl | TGGCAGGCGATCGCAATGT |
| | fwd | GCACTAAAATGGAACAGC |
| | | CCTTC |
| | | (SEQ ID NO: 28) |
| 29 | Sgfl-JunB-Sgsl | TAGTCTGGCGCGCCGAAGG |
| | rev | CGTGTCCCTTGA |
| | | (SEQ ID NO: 29) |
| 30 | Sgfl-IKZFl-Sgsl | CTGGCAGGCGATCGCAATG |
| | fwd | GATGCTGATGAGGGTCAA |
| | | GACATGTCCC |
| | | (SEQ ID NO: 30) |
| 31 | Sgfl-IKZFl-Sgsl | TAGTCTGGCGCGCCGCTCA |
| | rev | TGTGGAAGCGGTGCT |
| | | (SEQ ID NO: 31) |
| 32 | Sgfl-STATl-Sgsl | GCAGGCGATCGCAATGTCT |
| | | CAGTGGTACGAA |
| | fwd | (SEQ ID NO: 32) |
| 33 | Sgfl-STATl-Sgsl | CTGGCGCGCCTACTGTGTT |
| | rev | CATCATACTGTCG |
| | | (SEQ ID NO: 33) |

EPEA nanobody gene block sequence

(SEQ ID NO: 34)

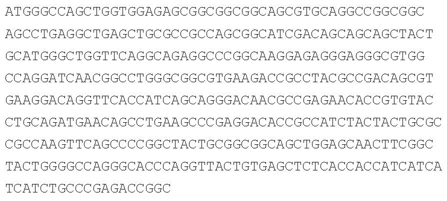

### Instrumentation

Organic compounds were characterized at the Nuclear Magnetic Resonance (NMR) Facility and High-Resolution Mass Spectrometry (HRMS) Facility in the Chemistry and Chemical Biology Department, Harvard University. Proton NMR spectra (¹H NMR) were recorded at 400 or 500 MHz at 24° C. Proton-decoupled carbon NMR spectra (¹³C NMR) were recorded at 125 MHz at 24° C. HRMS measurements were obtained using a Bruker microTOF-Q II hybrid quadrupoletime of flight, Agilent 1260 UPLC-MS. Low-resolution mass spectrometry (LRMS) measurements were obtained on Waters ACQUITY UPLC equipped with a SQ Detector 2 mass spectrometer. Protein quantification by bicinchoninic acid assay was measured on a multi-mode microplate reader FilterMax F3 (Molecular Devices LLC, Sunnyvale, Calif.). Cell lysis was performed using a Branson Ultrasonic Probe Sonicator (model 250). Fluorescence and chemiluminescence measurements were detected on an Azure Imager C600 (Azure Biosystems, Inc., Dublin, Calif.). All glycoproteomics data were obtained on a Waters ACQUITY UPLC connected in line to an Orbitrap Fusion Tribrid (ThermoFisher) within the Mass Spectrometry and Proteomics Resource Laboratory at Harvard University. Confocal fluorescence microscopy was performed at the Harvard Center for Biological Imaging (HCBI) using a Zeiss laser scanning confocal microscope (LSM) 880.

### Molecular Cloning Procedures

Plasmid #1 was a GFP nanobody fusion to full-length OGT developed by Gibson assembly and inserted into the pcDNA3.1 vector. The forward primer #1, used to amplify nGFP from cloning plasmid #1, contained an overlapping region to the pcDNA3.1 vector, Kozak sequence, a HA-tag for immunodetection, a Sgfl restriction enzyme (RE) site, and nucleotides complementary to the nGFP sequence. The reverse primer #2 contained complementary nucleotides to the nGFP sequence, a Sgsl RE site, and a stretch of nucleotides coding for a rigid helical linker composed of four iterations of the amino acid sequence EAAAK (SEQ ID NO: 43). The forward primer #3, used to amplify the OGT gene from cloning plasmid #2, included an overlapping region to the EAAAK (SEQ ID NO: 43) linker, a BamH1 RE site, and complementary nucleotides to the OGT gene. The reverse primer #4 for OGT contained complementary nucleotides to the C-terminus of the OGT gene, a Not1 RE site, and overlapping nucleotides to the pcDNA3.1 vector. The pcDNA3.1 vector was restriction enzyme digested with HindIII and NotI enzymes and a Gibson Assembly was performed to construct the HA-nGFP-OGT(13) plasmid #1.

Plasmids #2-4 were derived from plasmid #1 by restriction enzyme cloning by designing forward primers #5-7 containing a Sgfl RE site and complementary regions of interest in GFP, RFP, or nEPEA and reverse primers #8-10 containing a Sgfl RE site and complementary regions to the C-terminus of GFP, RFP, or nEPEA. PCR products were inserted into a Sgfl and Sgsl digested plasmid #1.

The OGT(13) plasmid #5 without the nanobody was created by designing a forward primer #11 containing a HindIII RE site, a HA tag, a BamHI RE site and complementary regions to OGT. The reverse primer #12 contained a NotI RE site and complementary regions to the C-terminus of OGT. PCR products were inserted into a HindIII and NotI digested pcDNA3.1 plasmid.

OGT(4) plasmids #6-9 were developed by restriction enzyme cloning by designing a forward primer #13 containing a BamHI RE site and complementary regions of interest in OGT and the reverse primer #12 containing a NotI RE site and complementary regions to the C-terminus of OGT. PCR products were inserted into BamHI and NotI digested plasmids #1, 2, 4 and 5.

OGT(K852A) plasmids #10 and 11 were developed by site-directed mutagenesis by designing forward primer #14 and reverse primer #15. Whole plasmid PCR products of plasmids #8 and 9 were obtained and blunt end cloning was performed.

GFP-Flag-JunB-EPEA plasmid #12 was developed by Gibson Assembly and inserted into the pcDNA3.1 vector. The forward primer #16, used to amplify GFP from cloning plasmid #3, contained an overlapping region to the pcDNA3.1 vector, a HindIII RE site, and nucleotides complementary to the GFP sequence. The reverse primer #17 contained complementary nucleotides to the GFP sequence, a Flag tag and one iteration of the amino acid sequence EAAAK. The forward primer #18, used to amplify the JunB gene from cloning plasmid #5 included an overlapping region to the Flag-EAAAK linker and complementary nucleotides to the JunB gene. The reverse primer #19 for JunB contained complementary nucleotides to the C-terminus of JunB, an EPEA tag, a XhoI RE site, and overlapping nucleotides to the pcDNA3.1 vector. The pcDNA3.1 vector was restriction enzyme digested with HindIII and XhoI enzymes and a Gibson Assembly was performed.

Nup62-Flag-EPEA plasmid #13 was developed by restriction enzyme cloning. A forward primer #20 containing a HindIII and Sgfl RE sites and a region complementary to the N-terminus of Nup62 was created. A reverse primer #21 with an XhoI RE site and regions complementary to the Flag and EPEA tag was created. The pcDNA3.1 vector was digested with the HindIII and XhoI restriction enzymes and restriction enzyme cloning was performed to develop the Nup62-Flag-EPEA plasmid #10. All other plasmids containing target proteins (plasmids #14-15) were created by designing forward and reverse primers containing either Sgfl or Sgsl RE sites and complementarity to the gene of interest and inserted into a Sgfl- and Sgsl-digested Nup62-Flag-EPEA plasmid #13.

Plasmid #16 was generated by restriction enzyme cloning using Sgsl and Sgfl enzymes on a pcDNA3.1-Nup62-Flag plasmid and plasmid #15. Digested products were ligated to produce plasmid #16.

Plasmids #11-21 were derived from a pcDNA3.1 vector containing Nup62 fused to a C-terminal Flag and EPEA tag (plasmid #10) developed by restriction enzyme cloning. A forward primer #13 containing a HindIII and Sgfl RE sites and a region complementary to the N-terminus of Nup62 was created. A reverse primer #14 with an XhoI RE site and regions complementary to the Flag and EPEA tag was created. The pcDNA3.1 vector was digested with the HindIII and XhoI restriction enzymes and restriction enzyme cloning was performed to develop the Nup62-Flag-EPEA plasmid #10.

The HA-nEPEA-OGT(13) plasmid #4 fusion was made from plasmid #1 by restriction enzyme cloning. The nEPEA sequence was obtained from a gene block (IDT). Forward primer #5 containing Sgfl and complementarity to the N-terminus of the EPEA nanobody and reverse primer #6 containing Sgsl RE sites and complementarity to the C-terminus of the EPEA nanobody were used for PCR. PCR products were inserted into a Sgfl- and Sgsl-digested HA-nEPEA-OGT(13) plasmid #4. All other plasmids containing OGT (Plasmids #2, 3, 5, 6) were developed by restriction enzyme cloning by designing forward primers containing a BamHI RE site and complementary regions of interest in OGT and a reverse primer contained a NotI RE site and complementary regions to the C-terminus of OGT. PCR products were inserted into either a BamHI and NotI digested plasmid #1 or #4. All other plasmids containing OGT without the nanobody (Plasmids #7-9) were created by restriction enzyme cloning into a pcDNA3.1-HA vector containing BamHI and NotI RE sites after the HA-tag. All other plasmids containing target proteins (Plasmids #11-21) were created by restriction enzyme cloning into the Nup62-Flag-EPEA plasmid #10. Forward and reverse primers containing either Sgfl or Sgsl RE sites and complementarity to the gene of interest were designed. PCR products were inserted into a Sgfl- and Sgsl-digested Nup62-Flag-EPEA plasmid #10.

### Generation of α-Synuclein CRISPR Knockout HEK Cell Line

The α-synuclein CRISPR/Cas9 KO plasmid (human, Cat #sc-417273) and α-synuclein homology-directed DNA repair (HDR) plasmid (human, Cat #sc-417273-HDR) were purchased from Santa Cruz Biotechnology and transfected following the manufacturer's instructions. The media was replaced with fresh DMEM growth media after 24 h. After 48 h of transfection, DMEM media supplemented with 2m/mL puromycin was added to the cells for KO-positive selection. The puromycin selection continued for 14 d with increasing concentration of puromycin up to 6m/mL prior to FACS to enrich for the RFP-positive cells (top 5% highest RFP intensity).

### Cell Culture, Transfection Protocols, and Cell Lysate Collection

At least some of the experiments were performed with HEK293T cells, α-syn KO HEK293 cells, or U2OS cells, unless otherwise noted. Cells were cultured in high-glucose with pyruvate Dulbecco's Modified Eagle Medium (DMEM, ref. 11995073) supplemented with 10% FBS and 1% penicillin—streptomycin at 37° C. in a humidified atmosphere with 5% CO₂, unless otherwise noted.

Samples for Western blot, biotin enrichment, or immunofluorescence were prepared from cells seeded in a well of a sterile 6-well plate (VWR, ref. 10062-892) at a density ˜1×10⁶ cells/well and transfected at ˜80% confluency the next day. For mass spectrometry-based glycoproteomics experiments, cells were seeded at the density of either ˜18×10⁶ cells/plate or ˜25×10⁶ cells/plate in a sterile 150 mm tissue culture dishes (Corning, ref. 25383-103) and transfected at ˜80% confluency the next day. Transient expression of the indicated proteins was performed by transfection with the desired plasmids following the manufacturer's protocol. For immunofluorescence experiments, Lipofectamine 2000 (ThermoFisher, ref. 11668027) was used at a ratio of 2 μg plasmid DNA to 5 μL of Lipofectamine. For all other experiments, TransiT-PRO (Mirus Bio, ref. MIR 5740) was used with a ratio of 1 μg plasmid DNA to 1 μL of TransiT-PRO. As recommended by the manufacturers, transfection reagent and plasmid were diluted in Opti-MEM reduced serum medium (ThermoFisher, ref. 31985070) during the transfection protocol. Cells were incubated for 36-48 h after transfection before collection or visualization.

After 36-48 h of transfection, cells were collected and lysed by probe sonication in lysis buffer [150 μL of 2% SDS+1×PBS+1× Protease inhibitors (cOmplete™, EDTA-free Protease Inhibitor Cocktail, Sigma Aldrich; cat #11873580001)]. A BCA assay was performed to determine protein concentration and the concentration was adjusted to 2.5 μg/μL with lysis buffer.

### Glycoprotein Enrichment Assay

Cell lysates (40 μL, 100 μg) were treated with a pre-mixed solution of Click chemistry reagents for a final volume of 150 μL (final concentrations: 1×PBS, 100 μM biotin-PEG4-alkyne, 2 mM sodium ascorbate, 100 μM THPTA, 1 mM CuSO₄) 1 h at 24° C. The reaction was quenched by the addition of methanol (1 mL) and the proteins were precipitated by incubating the mixture for 30 min at −80° C. Protein was pelleted by centrifugation (10 min, 21,130×g), the supernatant was discarded, and the resulting protein pellet was resuspended by probe tip sonication in 50 μL of 1% SDS+1×PBS.

A 50% slurry of streptavidin-agarose beads (Biovision, 40 μL) and 1×PBS (450 μL) were added to the resuspended proteins. The mixture was incubated with rotation for 1 h at 24° C. Beads were pelleted by centrifugation (1 min, 1,503×g). The beads were washed sequentially with the following solutions: 1×1 mL of 1% SDS in PBS, 2×1 mL of 6 M urea, 2×1 mL of 1×TBST. The washed beads were resuspended in 50 μL of 1× Laemmli sample buffer (final concentrations: 60 mM Tris-HCl, 2% SDS, 10% glycerol, 5% ß-mercaptoethanol, 0.01% bromophenol blue) and heated for 5 min at 95° C. before loading on a gel for Western blot analysis.

### PEG-5K Glycoprotein Labeling

Mass shift assays were performed according to the procedure of Pratt and co-workers (Butkinaree, C.; Park, K.; Hart, G. W. Biochimica et Biophysica Acta 2010, 180, 2010). Samples (200m) were reduced with 25 mM DTT and heated for 5 min at 95° C. Samples were then alkylated with 50 mM iodoacetamide for 1 h in the dark at 24° C. Samples were precipitated by the addition of methanol (600 μL), chloroform (200 μL), and water (450 μL), vortexing, and centrifugation (10 min, 10,000×g). The aqueous upper layer was discarded and methanol (1 mL) was added, sample was vortexed, and centrifuged (10 min, 10,000×g). Sample was allowed to air dry before resuspension in 2% SDS+1×PBS (45 μL) by probe tip sonication. Ten mM DBCO-PEG5K (5 μL, Click Chemistry Tools) was added and the solution warmed in a heat block for 5 min at 95° C. Samples were precipitated by the addition of methanol (600 μL), chloroform (200 μL), and water (450 μL), vortexing and centrifugation (10 min, 10,000×g). Aqueous upper layer was discarded and methanol (1 mL) was added, sample was vortexed, and centrifuged (10 min, 10,000×g). Sample was allowed to air dry before resuspension by probe tip sonication in 2% SDS+1×PBS (40 μL). 5× Laemmli sample buffer (10 μL) was added and the samples were heated for 5 min at 95° C. for Western blot analysis.

### Chemoenzymatic Labeling of O-GlcNAc by Y289L GalT1 Enzyme and Chemical Enrichment

Y289L GalT1 enzyme was expressed and purified following the procedure of Hsieh-Wilson and co-workers (Gambetta, M. C.; Muller, J. A Chromosoma 2015, 124, 429). Briefly, 2 mg of cell lysates (400 μL), which had been previously reduced and alkylated, were mixed with water (490 μL), GalT1 labeling buffer (800 μL, final concentrations: 50 mM NaCl, 20 mM HEPES, 2% NP-40, pH 7.9), and 100 mM MnCl2 (110 μL) were added in order. The sample was vortexed and transferred to ice. Then, 500 μM UDP-GalNAz (100 μL) and 2 mg/mL GalT1 enzyme (100 μL) were added to the sample. Subsequently, the sample reaction was rotated for 16 h at 4° C. Samples were precipitated by the addition of methanol (1.2 mL), chloroform (400 μL), and water (900 μL), vortexing and centrifugation (10 min, 10,000×g). Aqueous upper layer was discarded and methanol (1 mL) was added, sample was vortexed, and centrifuged (10 min, 10,000×g). Sample was allowed to air dry before resuspension in 2% SDS+1×PBS (400 μL). A pre-mixed solution of the click chemistry reagents (100 μL; final concentration of 200 μM IsoTaG silane probe, 500 μM CuSO4, 100 μM THPTA, 2.5 mM sodium ascorbate) was added and the reaction was incubated for 3.5 h at 24° C. Samples were precipitated by the addition of methanol (600 μL), chloroform (200 μL), and water (450 μL), vortexing and centrifugation (10 min, 10,000×g). Aqueous upper layer was discarded and methanol (1 mL) was added, sample was vortexed, and centrifuged (10 min, 10,000×g). Sample was allowed to air dry before resuspension in 2% SDS+1×PBS (400 μL) by probe tip sonication. Streptavidin-agarose resin [400 μL of the resin slurry, washed with PBS (3×1 mL)] was added, and the resulting mixture was incubated for 12 h at 24° C. with rotation. The beads were washed using spin columns with 8 M urea (5×1 mL), and PBS (5×1 mL). Washed beads were resuspended in 1×PBS+10 mM CaCl2 (520 μL). Fifty μL of this mixture was saved for analysis to determine protein enrichment and capture by Western blot. Eight M urea (32 μL) and trypsin (1.5 μg) was added to the beads and digestion was allowed to occur for 16 h at 37° C. with rotation. Beads were pelleted by centrifugation (3000×g, 3 min), and the supernatant digest was collected. The beads were washed with PBS (1×200 μL) and H₂O (2×200 μL). Washes were combined with the supernatant digest to form the trypsin digest. The IsoTaG silane probe was cleaved with 2% formic acid/water (2×200 μL) for 30 min at 24° C. with rotation and the eluent was collected. The beads were washed with 50% acetonitrile-water+1% formic acid (2×500 μL), and the washes were combined with the eluent to form the cleavage fraction. The trypsin digest and cleavage fraction were concentrated using a vacuum centrifuge (i.e., a speedvac, 40° C.) to dryness and then resuspended with 2% formic acid/water (50 μL). Samples were desalted with a ZipTip P10. Trypsin fractions were resuspended in 50 mM TEAB (20 μL) and TMT reagent (2 μL) was added to the samples and incubated for 1 h at 24° C. Hydroxyammonia (50%, 1 μL) was added to the samples to quench the reaction for 15 min at 24° C. Samples were combined and concentrated using a vacuum centrifuge (i.e., a speedvac) to dryness and stored at −20° C. until analysis.

### Western Blot Procedures

The protein sample (15 μL) was loaded on 6-12% or 6-10% Tris-Glycine SDS-PAGE gels and ran on a Mini-PROTEAN® BioRad gel system. Gels were transferred with the Invitrogen iBlot. For α-synuclein blots, membranes were incubated in 1% paraformaldehyde for 1 hr to prevent α-synuclein dissociation from the membrane as previously described prior to blocking.24 Membranes were stained with LI-COR Revert total protein stain to verify transfer and equal protein loading and blocked with 3% BSA+1×TBST for 1 h at 24° C. Primary antibodies and the following dilutions were incubated with the membranes for 1 h to 12 h: anti-Flag (1:5,000; Sigma Aldrich; Cat #F3165), anti-Flag (1:1,000; Cell Signaling; Cat #14793S); anti-HA (1:1,000; Cell Signaling; Cat #3724S) anti-O-GlcNAc RL2 (1:1,000; Abcam; Cat #ab2739), anti-synuclein (1:1,000; Abcam; Cat #ab138501). Membranes were washed 3×5 min each wash with 1×TBST and incubated with the following secondary antibodies and dilutions: anti-Mouse HRP (1:10,000; Rockland Immunochemicals: Cat #610-1302), anti-Rabbit HRP (1:10,000; Rockland Immunochemicals: Cat #611-1302), anti-Mouse IR 800 (1:10,000; LI-COR; Cat #925-32210), anti-Rabbit IR 680 (1:10,000; LI-COR; Cat #925-68071), anti-Rabbit IR 800 (1:10,000; LI-COR; Cat #925-32211).. Membranes were washed 3×5 min each wash with 1×TBST and results obtained by chemiluminescence or IR imaging using the Azure c600. Membranes were quantified using LI-COR image studio lite.

### EPEA-Tag Immunoprecipitation

For EPEA-tag immunoprecipitation and Western blot, α-syn KO HEK293T cells transfected in a 6-well plate were collected in lysis buffer [150 μL of 2% SDS+1×PBS+50 μM Thiamet-G+1×protease inhibitors (cOmplete™, EDTA-free Protease Inhibitor Cocktail, Sigma Aldrich; Cat #11873580001)]. Samples were heated for 5 min at 95° C. and lysed by probe tip sonication 10 secs 10% amplitude. A BCA assay was used to determine protein concentration and the concentration was adjusted to 2.5 μg/μL with lysis buffer. Protein Lysate (100 μg) was incubated with C-tag resin (40 μL, Thermo Fisher; Cat #191307005) and 1×PBS (500 μL). The mixture was incubated 12 h at 4° C. The beads were washed 5× with 1×TBST (1 mL) and resuspended in 1× Laemmli sample buffer (50 μL; final concentrations: 60 mM Tris-HCl, 2% SDS, 10% glycerol, 5% ß-mercaptoethanol, 0.01% bromophenol blue) and heated for 5 min at 95° C. before loading on a gel for Western blot analysis.

For target protein glycoproteomics, α-syn KO HEK293 cells were plated in a 150-mm dish with the corresponding plasmids for 48 h. Cells were collected in 2% SDS+1×PBS+1× Protease inhibitors+50 μM Thiamet-G (2 mL) and heated for 5 min at 95° C. Cells were lysed by probe tip sonication (30 sec, 15% amplitude). Samples were reduced with 25 mM DTT and heating for 5 min at 95° C. Samples were then alkylated with 50 mM iodoacetamide for 1 h in the dark at 24° C. Samples were precipitated by the addition of methanol (1.2 mL), chloroform (400 μL), and H₂O (900 μL), vortexing, and centrifugation (10 min, 10,000×g). Aqueous upper layer was discarded and methanol (1 mL) was added, sample was vortexed, and centrifuged (10 min, 10,000×g). Sample was allowed to air dry (5 min) before resuspension in 2% SDS+1×PBS (500 μL) by probe tip sonication. A BCA assay was performed and protein concentration was adjusted to 5 μg/μL with lysis buffer. Protein lysate (2.5 mg) was incubated with C-tag XL (300 Thermo Fisher; Cat #2943072005) and 1×PBS (1 mL). The mixture was incubated 12 h at 4° C. 50 μL of this mixture was saved for analysis to determine protein enrichment and capture by Western blot. The beads were washed with 10× with 1×PBS, then the beads were resuspended in 100 mM Tris-HCl+10 mM CaCl₂) (pH 8.0, 520 μL) for chymotrypsin digestion. 8 M urea (32 μL) was added and chymotrypsin (2 μg) was added to the beads and digestion was allowed to occur for 16 h at 24° C. Beads were pelleted, and supernatant was transferred to a new tube. Beads were washed 3× with 1×PBS (200 μL) and the washes were transferred to the supernatant tube. Sample was concentrated to dryness using a vacuum centrifuge (i.e., a speedvac). Samples were desalted with a ZipTip P10, concentrated to dryness, and stored at −20° C. until analysis.

### Mass Spectrometry Acquisition Procedures

Desalted samples were reconstituted in 0.1% formic acid in water (20 μL), and half of the sample (10 μL) was injected onto a C18 trap column (WATERS cat #186008821 nanoEase MZ Symmetry C18 Trap Column, 100 Å, 5 μm×180 μm×20 mm) and separated on an analytical column (WATERS cat #186008795 nanoEase MZ Peptide BEH C18 Column, 130 Å, 1.7 μm×75 μm×250 mm) with a Waters nanoAcquity system connected in line to a ThermoScientific Orbitrap Fusion Tribrid. The column temperature was maintained at 50° C. Peptides were eluted using a multi-step gradient at a flow rate of 0.15 μL/min over 120 min (0-5 min, 2-5% acetonitrile in 0.1% formic acid in water; 5-95 min, 5-50%; 95-105 min, 50-98%; 105-115 min, 98%; 115-116 min, 98-2%; 116-120 min, 2%). The electrospray ionization voltage was set to 2 kV and the capillary temperature was set to 275° C. Dynamic exclusion was enabled with a repeat count of 2, repeat duration of 30 s, exclusion list size of 400, and exclusion duration of 30 s. MS1 scans were performed over 400-2000 m/z at resolution 120,000 and the top twenty most intense ions (+2 to +6 charge states) were subjected to MS2 HCD fragmentation at 27%, for 75 ms, at resolution 50,000. Other relevant parameters of HCD include: isolation window (3 m/z), first mass (100 m/z), and inject ions for all available parallelizable time (True). If oxonium product ions (138.0545, 204.0867, 345.1400, 347.1530, 366.1396, 507.1930, or 509.2060 m/z) were observed in the HCD spectra, ETD (250 ms) with supplemental activation (35%) was performed in a subsequent scan on the same precursor ion selected for HCD. Other relevant parameters of ETHCD include: isolation window (3 m/z), use calibrated charge-dependent ETD parameters (True), Orbitrap resolution (50 k), first mass (100 m/z), and inject ions for all available parallelizable time (True).

### Mass Spectrometry Data Analysis

The raw data was processed using Proteome Discoverer 2.3 (Thermo Fisher Scientific). For quantitative proteomics and global glycoproteomics, the data was searched against the human-specific SwissProt-reviewed database 2016 (20,152 proteins, downloaded on Aug. 19, 2016). For immunoprecipitated samples for glycoproteomics, the data were searched against the target protein sequence (Nup62, P37198; JunB, P17275; TET3, 043151), chymotrypsin, trypsin, the HA-nEPEA-OGT construct, and alpha-synuclein. For quantitative proteomics, analysis was performed in Thermo Scientific Proteome Discoverer version 2.3. HCD spectra with a signal-to-noise ratio greater than 1.5 were searched against a database containing the Swissprot 2016 annotated human proteome and contaminant proteins using Sequest HT with a mass tolerance of 10 ppm for the precursor and 0.02 Da for fragment ions with specific trypsin digestion, 2 missed cleavages, variable oxidation on methionine residues (+15.995 Da), static carboxyamidomethylation of cysteine residues (+57.021 Da), and static TMT labeling (229.163 Da) at lysine residues and peptide N-termini. Assignments were validated using Percolator. The resulting assignments were filtered to only include high-confidence matches, and TMT reporter ions were quantified using the Reporter Ions Quantifier and normalized such that the summed peptide intensity per channel was equal. For all glycoproteomics data, the data was searched using Byonic v3.0.0 as a node in Proteome Discoverer 2.3 for glycopeptide searches. Indexed databases for either tryptic or chymotryptic digests were created with full cleavage specificity. The database allowed for up to three missed cleavages with variable modifications (methionine oxidation, +15.9949 Da; carbamidomethylcysteine, +57.0215 Da; deamidation of asparagine and glutamine, +0.984016 Da; and others as described below). Precursor ion mass tolerances for spectra acquired using the Orbitrap were set to 10 ppm. The fragment ion mass tolerance for spectra acquired using the Orbitrap were set to 20 ppm. For global glycoproteomics, glycopeptide searches allowed for tagged 0-glycan variable modifications (HexNAcHexNAzSi0+547.2128, HexNAcHexNAzSi2+549.2251, HexNAc+203.0794, on serine, threonine and cysteine). For immunoprecipitated samples for glycoproteomics, glycopeptide searches allowed for tagged HexNAc modifications (HexNAc+203.0794 on serine, threonine) and methionine oxidation (+15.9949 Da). Glycopeptide spectral assignments passing a false discovery rate (FDR) of 1% at the spectrum peptide match level based on a target decoy database were manually validated. 0-Glycosites were considered an unambiguous glycosite if the glycosite was identified in two independent PSMs based on the presence of one serine or threonine in the peptide or if the assignment derived from an EThcD spectrum with Byonic delta modification score larger than 10.

### Immunofluorescence and Fixed-Cell Sample Preparation

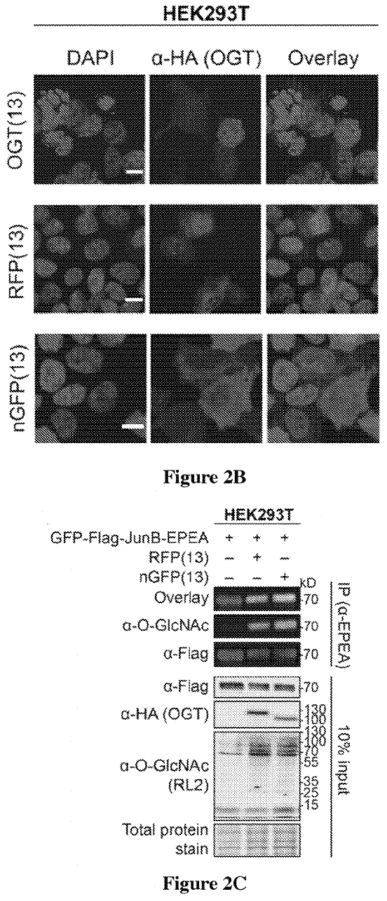

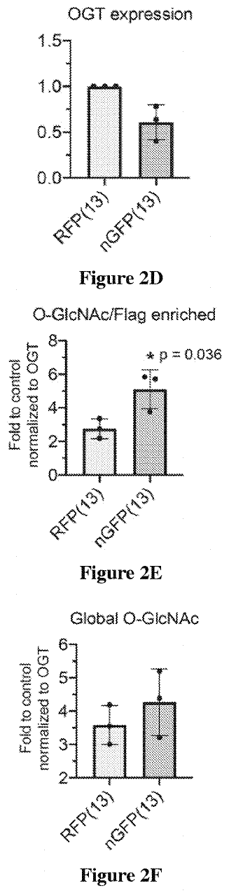

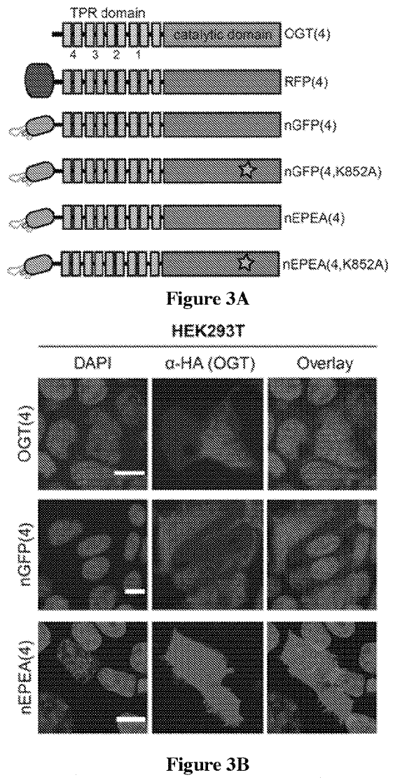

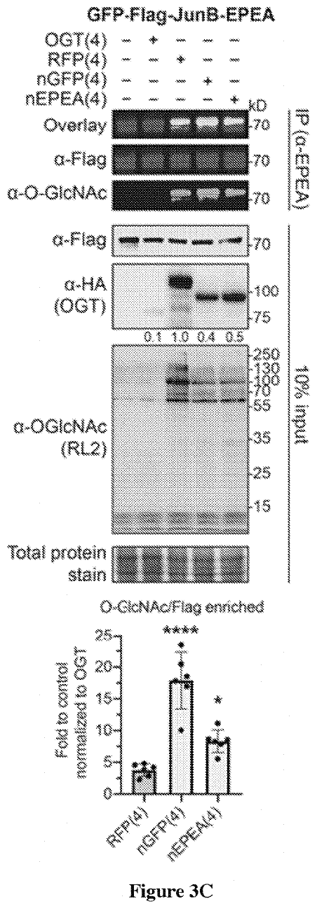

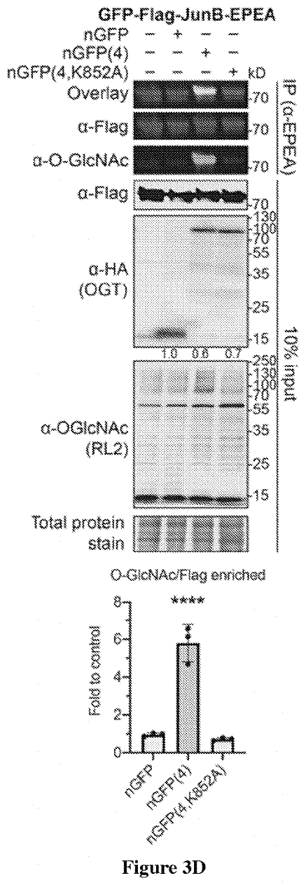

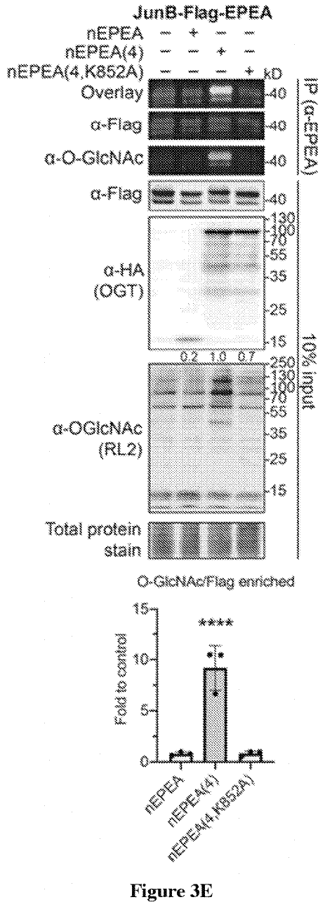

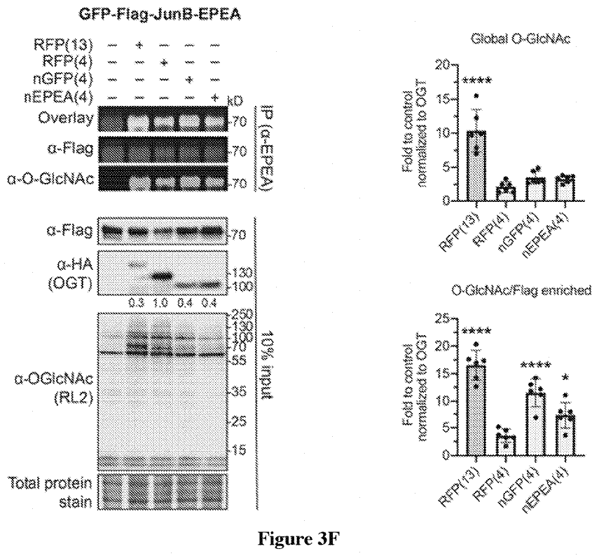

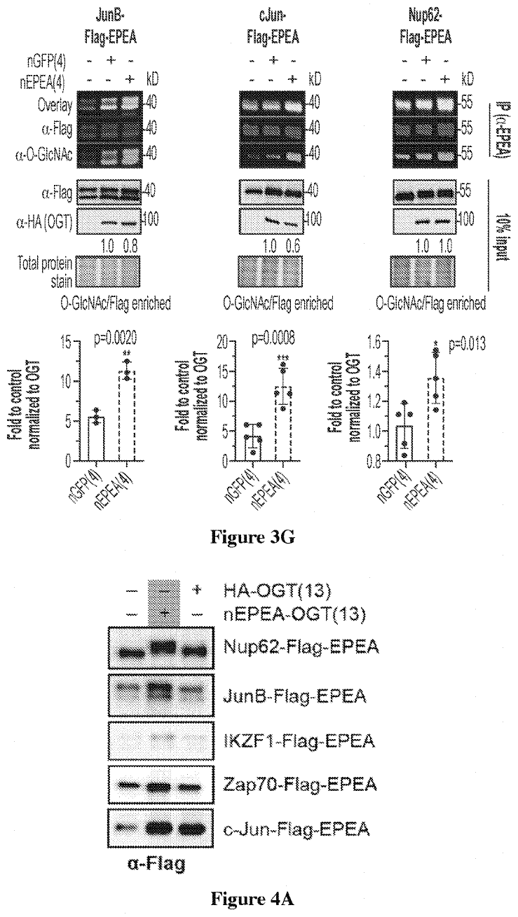

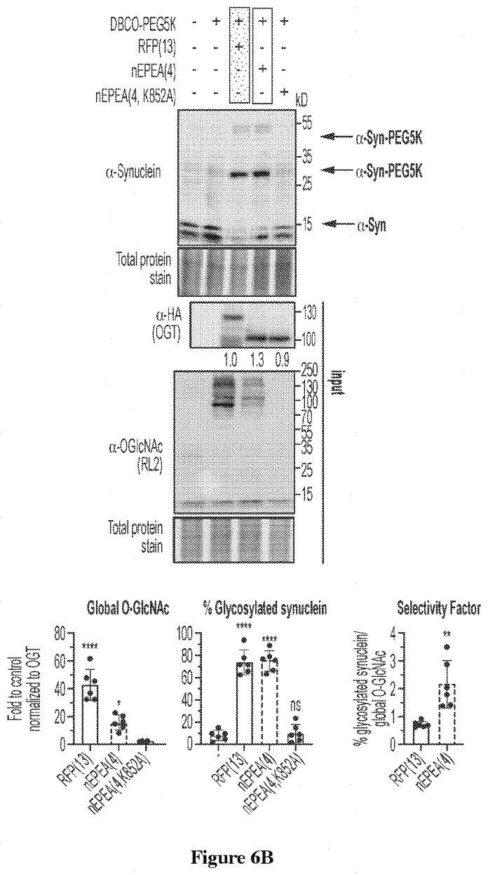

Cells were seeded on 22×22 mm glass coverslips no. 1.5 coated with poly-L-lysine (Neuvitro Corporation German Glass Coverslips ref. H-22-1.5-pII) that had been placed in single wells of a 6-well plate for 24 h prior to transfection. For experiments in FIG. 6A, cells were plated in normal-glucose (1 g/L) DMEM (Corning, ref. 10014CV). Cells were transfected for 48 h and the media were exchanged with fresh media after 24 h. For experiments in FIG. 6A, cells were transfected in a 6-well plate without coverslips for 24 h, trypsinized and replated on a glass coverslip mentioned above. The replated cells were kept for an additional 24 h. Transfected cells were fixed in freshly prepared 4% paraformaldehyde in PBS (pH 7.4) for 15 min at 24° C. (1 mL per well), washed with PBS (2 mL per well) twice (10 min), permeabilized in PBS with 0.1% Triton X-100 (1 mL per well) for 20 min at 24° C. Cells were washed with PBS for 15 min (3×2 mL), and then incubated with blocking solution (3% BSA/TBST) for at least 1 h at 4° C., followed by overnight incubation with the primary antibody. Cells were washed with PBS for 15 min (3×2 mL), and subsequently incubated with the secondary antibody for 1 h at 4° C., washed with PBS for 15 min (3×2 mL). The nuclei were stained in DAPI solution (4′,6 diamidino-2-phenylindole, Invitrogen Molecular Probes NucBlue, ref. R37606) for 10 min at 24° C. Coverslips were mounted in anti-fade Diamond (Life Technologies ref. P36961). Primary antibodies were mouse anti-Flag mAb (1:1000, FLAG-M2, Sigma-Aldrich, ref. F3165-.2MG) and rabbit anti-HA mAb (1:1000, HATag-C29F4 Cell Signaling, ref. 3724S). Secondary antibodies were goat anti-Mouse IgG (H+L) Cross-Adsorbed Secondary Antibody, Alexa Fluor 488 (1:5000, ThermoFisher/Invitrogen, ref. PIA28175) and goat anti-Rabbit IgG (H+L) cross-adsorbed secondary antibody conjugated to Alexa Fluor 594 (1:5000, ThermoFisher/Invitrogen, ref. A11012). The anti-HA images for α-synuclein KO HEK293 cells and U2OS cells in FIGS. 2 and 3 were obtained using the mouse antibody (mAb) for HA-Tag (6E2) conjugated with AlexaFluor647 (1:500, Cell Signaling, Cat #3444S).

### Confocal Fluorescence Microscopy, Image Acquisition and Processing

Fixed-cell samples were imaged using a Zeiss laser scanning confocal microscope (LSM) 880 confocal microscope. Images were acquired with a Plan-Apochromat 40× or 63×/1.4NA oil immersion objective DIC M27 (the magnification was adjusted by zooming in or out as needed). Excitation wavelengths for DAPI, Alexa Fluor488, red fluorescent protein (RFP), and Alexa Fluor594 were at 405 nm, 488 nm, 561 nm, and 594 nm, respectively. The laser power and detector gain were adjusted to obtain the best signal-to-noise ratio and have no over-saturated signal. Fluorescence was detected using the Zeiss QUASAR detection unit. Sequential Z stacks were acquired consisting of 11 planes separated by 0.5 μm, pixel size 0.19 μm, with a 0.52 μs pixel dwell time (2×2 averaging per frame was used). A pinhole size of 1 Airy Unit (AU) at all wavelengths was used. Images were processed with ImageJ2 (Fiji). All images shown are average-intensity projections from all slices in z-stacks.

### Statistical Analyses

Glycosites in EThCD spectra passing a 1% FDR and possessing a delta glycomod of greater than or equal to ten, or glycosites in peptides with only one potential site of modification, were considered confidently localized (“unambiguous” glycosites). All other glycopeptides passing a 1% FDR were considered “ambiguous” glycosites. Statistical analyses methods are described with the figures. Two tailed t-tests and one-way ANOVA tests were performed.

### Example 1: Design of Nanobody-OGT Fusion Proteins

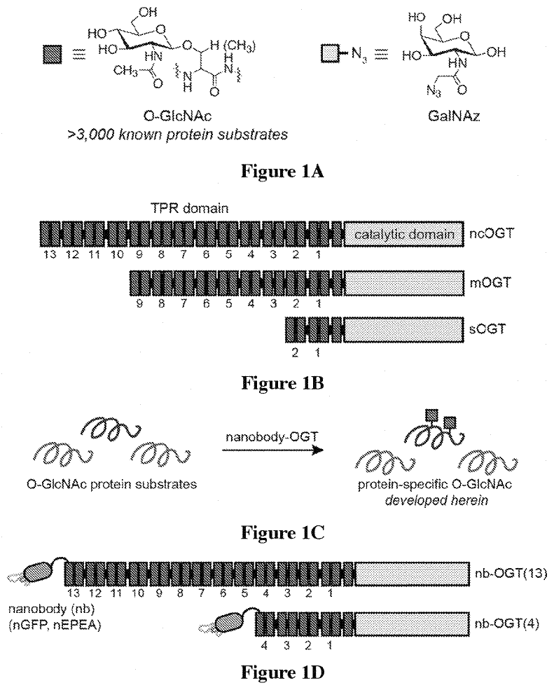

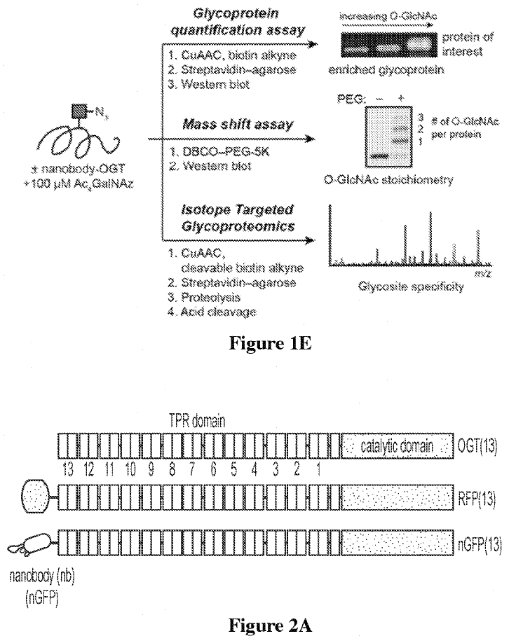

A series of nanobody-OGT fusion proteins were designed (FIG. 1A to 1D). Several fusion proteins were evaluated. Two of these were nGFP fused to the full-length OGT that possesses 13 TPRs [residues 1-1046, nGFP(13), also referred to as HA-nGFP-OGT(13)] and an RFP fusion to full-length OGT [RFP(13)] as an untargeted control for comparison to the nanobody-OGT construct (FIG. 2A). All fusions to OGT were connected by a common rigid linker (EAAAK)₄. It was found that expression of OGT(13) and RFP(13) was distributed throughout the cytoplasm and nucleus by using confocal fluorescence microscopy (FIG. 2B). The nGFP(13) construct was distributed throughout the nucleocytoplasmic space in an analogous manner.

Fusion proteins with a reduction in the TPR domain of OGT were also tested. One such fusion protein was nGFP fused to OGT that possesses 4 TPRs [residues 327-1046, nGFP(4), also referred to as HA-nGFP-OGT(4)]. A fusion to an additional nanobody, nEPEA(4) was also evaluated (FIG. 3A). The nEPEA nanobody was originally developed against α-synuclein and recognizes the four amino acid EPEA tag at the C-terminus of proteins. (De Genst, E. J. et al. Structure and properties of a complex of alpha-synuclein and a single domain camelid antibody. J Mol Biol 402, 326-343 (2010)). The EPEA tag sequence cannot be glycosylated itself and is minimally perturbative to protein structure. Because α-synuclein is found in HEK293 cells, a CRISPR KO α-synuclein cell line was generated for studies employing the EPEA nanobody. Expression of the OGT(4) fusion proteins in HEK293T cells showed a subcellular localization throughout the nucleocytoplasmic space by confocal fluorescence microscopy (FIG. 3B).

### Example 2: Targeting of GFP-c-JUN

The proximity-directing ability was tested in HEK293T cells co-transfected with GFP-Flag-JunB-EPEA, a transcription factor carrying multiple O-GlcNAc sites with emerging functions in regulation of JUN (Woo, C. M.; Lund, P. J.; Huang, A. C.; Davis, M. M.; Bertozzi, C. R.; Pitteri, S. J. Molecular & cellular proteomics: MCP 2018, 17, 764; Gia, Y.; Zhang, X.; Zhang, Y.; Wang, Y.; Xu, Y.; Liu, X.; Sun, F.; Wang, J.; Diabetes 2016, 65, 619; Kim, S.; Maynard, J. C.; Strickland, A.; Burlingame, A. L.; Milbrandt, J. Proceedings of the National Academy of Sciences 2011, 108, 3141). To measure the changes in O-GlcNAc on a target protein, cells were labeled with 100 μM Ac₄GalNAz, a metabolic reporter of protein O-GlcNAc. Installation of the chemical reporter for O-GlcNAc through metabolic or chemoenzymatic labeling enabled installation of a reporter molecule using copper-catalyzed azide-alkyne cyclo addition (CuAAC). The reporter molecule facilitates glycan-specific enrichment and quantification by Western blot, determination of O-GlcNAc protein occupancy by mass-shift PEG-5 kDa assays (Rexach, J. E.; Rogers, C. J.; Yu, S.; Tao, J.; Sun, Y. E.; Hsieh-Wilson, L. C. Nature Chemical Biology 2010, 6, 645), or glycosite assignment by mass spectrometry (FIG. 1D) (Woo, C. M.; lavarone, A. T.; Spiciarich, D. R.; Palaniappan, K. K.; Bertozzi, C. R. Nature Methods 2015, 12, 561). To perform the glycoprotein quantification assay, azide-labeled cell lysates transfected with different OGT constructs were tagged with a biotin-alkyne probe via CuAAC and affinity enriched on streptavidin-agarose.

Immunoprecipitation of GFP-Flag-JunB-EPEA and probing for O-GlcNAc revealed an increase in O-GlcNAc levels on the target protein that was dependent on the co-transfected nGFP(13) (FIG. 2C). The O-GlcNAcylated target protein was significantly increased when coexpressed with nGFP(13) as compared to RFP(13) (FIGS. 2D and 2E). However, global O-GlcNAc levels were equivalently elevated in the presence of RFP(13) and nGFP(13), implying that although nGFP(13) elevated levels of the O-GlcNAcylated target protein JunB, the selectivity for the target protein could be further improved (FIG. 2F).

The activities of OGT(4), RFP(4), nGFP(4) and nEPEA(4) were evaluated against the same target protein GFP-Flag-JunB-EPEA (FIG. 3C). Both nGFP(4) and nEPEA(4) significantly increased the OGlcNAcylated target protein JunB relative to the untargeted controls OGT(4) and RFP(4). Increased levels of O-GlcNAcylated JunB were installed directly from the active nGFP(4), but not with the nanobody nGFP alone or a catalytically inactive mutant nGFP(4,K852A) (FIG. 3D).

Similarly, levels of O-GlcNAcylated JunB were specifically increased in the presence of co-expression of nEPEA(4) but not in the presence of nEPEA or the catalytically inactive nEPEA(4,K852A) (FIG. 3E). The O-GlcNAcylation activity of the OGT(4) fusions were further compared to the full-length RFP(13) (FIG. 3F). The truncation of the TPR domain found in OGT(4) attenuated the increase in global O-GlcNAc levels observed with RFP(13). Use of the nanobody for proximity direction was found to selectively reinstate O-GlcNAc activity for the desired target protein (FIG. 3F).

The nanobody-OGT(4) system was further evaluated for the ability to selectively increase the O-GlcNAcylated target protein against three targets: JunB-Flag-EPEA, cJun-Flag-EPEA, and Nup62-Flag-EPEA in HEK293T cells. Using the three EPEA-tagged target proteins, the O-GlcNAcylated target protein was found to significantly increase under proximity-direction of the matched nEPEA(4), but not the mismatched nGFP(4) (FIG. 3G). Collectively, these data suggest an increase in selective O-GlcNAcylation by replacing elements of the TPR domain with the nanobody and the modular ability of the nanobody-OGT(4) to increase O-GlcNAc levels using GFP- or EPEA-tagged target proteins.

### Example 3: System Modularity

A nanobody that recognized specific peptide tags (Mutldermans, S. Annual Review of Biochemistry 2013, 82, 775) such as nEPEA which recognizes the four-amino acid EPEA tag was used to generate other fusion proteins (De Genst, E. J.; Guilliams, T.; Wellens, J.; O'Day, E. M.; Waudby, C. A.; Meehan, S.; Dumoulin, M.; Hsu, S. T.; Cremades, N.; Verschueren, K. H.; Pardon, E.; Wyns, L.; Steyaert, J.; Christodoulou, J.; Dobson, C. M. Journal of Molecular Biology 2010, 402, 326).

Substitution of nGFP with nEPEA afforded the two HA-nEPEA-OGT constructs from the full-length [HA-nEPEA-OGT(13)] and a partially truncated TPR domain [HA-nEPEA-OGT(4)]. Further, nEPEA was fused to OGT with a fully removed TPR domain [HA-nEPEA-OGT(0)]. The fusion proteins were transiently expressed in U2OS cells and their subcellular localization and global O-GlcNAc levels were determined by confocal microscopy. All of the OGT fusions with or without nEPEA were found throughout the nucleocytoplasmic space of U2OS cells. Over-expression of HA-OGT(13) and nEPEA-OGT(13) increased global O-GlcNAc levels, while elevated expression of partial or full reduction of the TPR domain on OGT did not alter global O-GlcNAc levels by confocal microscopy. Likewise, global O-GlcNAc levels were broadly unperturbed by expression of nEPEA-OGT(4) or nEPEA-OGT(0) by O-GlcNAc Western blot. Isotope targeted glycoproteomics (IsoTaG) were used to analyze the global glycosite shifts in the O-GlcNAc proteome (Woo, C. M.; lavarone, A. T.; Spiciarich, D. R.; Palaniappan, K. K.; Bertozzi, C. R. Nature Methods 2015, 12, 561). Cellular lysates following transfection with a HA-nEPEA-OGT construct were collected and chemoenzymatically labeled to introduce an azido-sugar for enrichment, isotopic recoding, and glycosite mapping by targeted mass spectrometry. nEPEA-OGT(13) showed the greatest enrichment of glycopeptides over the control [258/113 peptide spectral matches (PSMs), 228%], while nEPEA-OGT(4) exhibited a modest increase in glycopeptide PSMs (179/113 PSMs, 158%), and the fully truncated nEPEA-OGT(0) showed a decrease in PSMs relative to the control (100/113 PSMs, 88%). Additionally, the subcellular localization of the fusion proteins in HEK293T cells was evaluated, and it was found that the nEPEA-OGT(13) and nEPEA-OGT(0) fusions were localized in the cytoplasm, while nEPEA-OGT(4) was broadly distributed throughout the nucleocytoplasmic space.

### Example 4: Selectivity of nEPEA-OGT Constructs

A library of EPEA-tagged target proteins based on proteins determined as possessing significant O-GlcNAc stoichiometry was developed to analyze the scope of two proximity-directed nEPEA-OGT constructs (Woo, C. M.; Lund, P. J.; Huang, A. C.; Davis, M. M.; Bertozzi, C. R.; Pitteri, S. J. Molecular & cellular proteomics 2018, 17, 764). Plasmids encoding a total of eleven C-terminal EPEA-tagged proteins were prepared and co-transfected with a HA-nEPEA-OGT fusion protein. Targets represented the broad classes of protein substrates from which OGT normally selects: transcription factors (c-JUN, JUNB, IKZF1, STAT1), kinases (Zap70), oxidoreductase (TET3), the nucleoporins (Nup35, Nup62), and the nucleosome (H2B, H3, H4). Transfected cells were additionally metabolically labeled with Ac₄GalNAz and the O-GlcNAc stoichiometry on the target protein was visualized by glycoprotein quantification assay or mass shift assay.

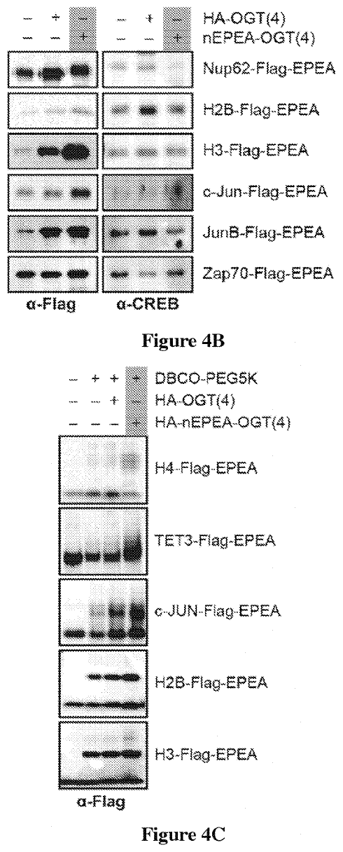

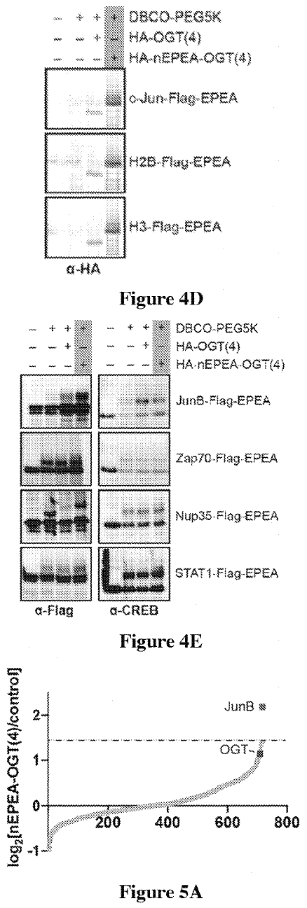

In all evaluated target proteins, co-transfection with HA-nEPEA-OGT(13) or HA-nEPEA-OGT(4) increased O-GlcNAc stoichiometry on the target protein. The full-length HA-nEPEA-OGT(13) increased O-GlcNAc stoichiometry to the evaluated proteins, above both control and coexpression of HA-OGT(13) samples by glycoprotein quantification assay (FIG. 4A). Increases in O-GlcNAc were observed on Nup62, JunB, and Zap70. Glycosites that had been previously mapped to each of these proteins reflect a large disparity in the homology sequence, which indicated that fusion of a nanobody to OGT promoted target protein recognition for a broad diversity of protein substrates and glycopeptide sequences.

Furthermore, HA-nEPEA-OGT(4) uniformly and selectively increased O-GlcNAz levels to all evaluated proteins (FIG. 4B). O-GlcNAz stoichiometry increased the most significantly on c-JUN and JunB from these assays. With H3 and JunB, an increase in O-GlcNAz was observed with the TPR truncated HA-OGT(4), which was induced further under nanobody-direction by the HA-nEPEA-OGT(4) construct. To control for enrichment loading, variance in OGT expression, and characterize substrate selectivity, CREB, an endogenous orthogonal 0GlcNAcylated protein in the nucleus, was visualized from the same experiment. In all examples, the abundance of CREB from an azideependent enrichment was equal or reduced compared to control lanes, which indicated that proximity direction was specific to the target protein and not globally increasing O-GlcNAc levels, in line with the minor shifts in O-GlcNAc observed by confocal microscopy, Western blot, and mass spectrometry.

A mass shift assay, where O-GlcNAz modifications on the proteome were labeled with a 5-kDa polyethylene glycol (DBCO-PEG5K) mass tag, was used to independently corroborate the glycoprotein enrichment assay and further revealed increases in O-GlcNAc stoichiometry (FIGS. 4C and 4E). Whole cell lysates from each of the transfected samples were treated with 100 μM DBCO-PEG5K and analyzed by immunoblotting to detect shifts in electrophoretic mobility of the target protein. The PEG mass tag introduced a discrete 5-kDa shift for every labeled O-GlcNAz group, which further reported the number of O-GlcNAz modifications per protein. The intensity of the mass-shifted bands relative to the native protein band provided an approximation of the O-GlcNAc stoichiometry. Increased O-GlcNAc stoichiometry on the target protein was observed in the presence of HA-nEPEA-OGT(4), analogous to results from biotin-based affinity enrichment (FIGS. 4C and 4E). Most glycoproteins displayed a single O-GlcNAc modification per molecule, including c-JUN, H2B, H3, H4, and TET3 irrespective of cotransfection with HA-OGT(4) or HA-nEPEA-OGT(4) (FIGS. 4C and 4D). Likewise, JunB, Zap70, Nup35, and STAT1 possessed one glycosite per molecule on average that increased in the presence of HA-nEPEA-OGT(4), and no major shift in O-GlcNAc stoichiometry was observed on endogenous CREB (FIG. 4E). The ability of HA-nEPEA-OGT(4) to redirect glycosyltransferase activity was successfully demonstrated against the 11 evaluated proteins that represent O-GlcNAc proteins from all parts of the proteome.

### Example 5: Quantitative Proteomics with Expressed Nanobody-OGT Constructs

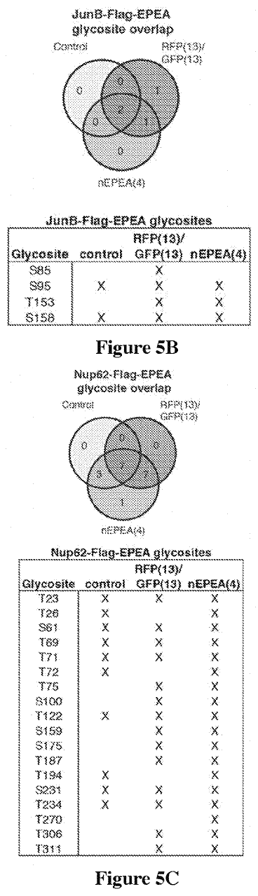

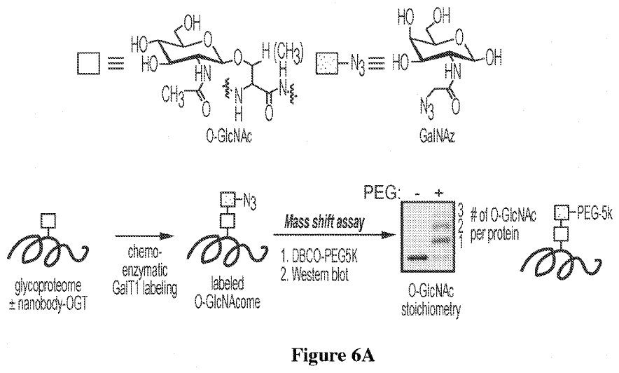

Quantitative proteomics experiments by mass spectrometry (MS) were conducted to quantify the selectivity of the nanobody-OGT constructs for the target protein. Cellular lysates were collected following co-expression in α-syn KO HEK293 cells of a HA-nEPEA-OGT construct with JunB-Flag-EPEA as the target protein. Lysates were subsequently chemoenzymatically labeled with UDP-GalNAz to introduce an azido-sugar for copper-catalyzed azide-alkyne cycloaddition (CuAAC) with a biotin-azide probe and enrichment on streptavidin-agarose beads. (Thompson, J. W., Griffin, M. E. & Hsieh-Wilson, L. C. in Meth Enzymol Vol. 598 (ed Barbara Imperiali) 101-135 (Academic Press, 2018).) The O-GlcNAcylated proteins were digested on-bead and labeled with Tandem Mass Tags (TMT) for MS analysis. Glycoprotein enrichment was determined relative to the control for high-confidence proteins [number of unique peptides ≥2, 1% false discovery rate (FDR)] (FIG. 5A). These data show that while JunB-Flag-EPEA was enriched by expression of HA-RFP-OGT(13), OGT itself was also enriched at nearly the same levels. By contrast, JunB-Flag-EPEA was the only protein enriched in samples co-expressing nEPEA(4), and overall enrichment of OGT itself was lower (highlighted as JunB and OGT, respectively, FIG. 5A).

### Example 6: Characterization of Glycosites Produced by Proximity-Directed OGT Constructs

In order to characterize the protein regions and the associated glycosites installed by the nanobody-OGT construct, JunB-Flag-EPEA was co-expressed with RFP/GFP(13) or nEPEA(4) in α-syn KO HEK293 cells. The proteins were affinity purified, digested with chymotrypsin, and analyzed by MS. Where possible, confident glycosites were filtered based on previously established criteria. (Woo, C. M. et al. Mapping and Quantification of Over 2000 O-linked Glycopeptides in Activated Human T Cells with Isotope-Targeted Glycoproteomics (Isotag). Mol Cell Proteomics 17, 764-775 (2018).) Four confident glycosites were identified from JunB-Flag-EPEA. Three of the four unambiguous glycosites were identified in at least one nanobody-OGT sample [nEPEA(4)] and one baseline sample [control, RFP/GFP(13)] (FIG. 5B). One glycosite S85 was identified only in the RFP/GFP(13) control indicating this site might only be accessible to a full-length OGT. Glycosite T153 was identified only under OGT overexpression conditions. This data suggest that the truncated nanobody-OGT construct targets similar glycosites as a full-length OGT.

The limits of the glycosite specificity of the nEPEA(4) construct were also evaluated on the highly O-GlcNAcylated protein Nup62. A total of 18 confident glycosites were mapped to Nup62-Flag-EPEA (FIG. 5C). Of the 18 unambiguously localized glycosites found on Nup62-Flag-EPEA, 17 glycosites were found in at least one of the baseline samples. The remaining glycosite was observed as a glycosite in nEPEA(4) (T270). We additionally observed several glycosites (T75, T100, S159, S175, T187, T306, T311) on Nup62-Flag-EPEA present only under OGT overexpression conditions. Taken together, the HA-nEPEA-OGT(13) and HA-nEPEAOGT(4) displayed analogous glycosite selectivity towards Nup62-Flag-EPEA and JunB-Flag-EPEA while increasing overall O-GlcNAc levels to the target protein.

To further measure specificity of the proximity-directed OGT, cells transfected with the empty vector or the catalytically attenuated HA-nEPEA-OGT(13)^{H498A} were prepared in parallel. The high O-GlcNAc stoichiometry produced a visible mass shift in Nup62 for direct estimation of O-GlcNAc levels by Western blot, although only six O-GlcNAc sites had been previously identified on Nup62 (Woo, C. M.; Lund, P. J.; Huang, A. C.; Davis, M. M.; Bertozzi, C. R.; Pitteri, S. J. Molecular & cellular proteomics: MCP 2018, 17, 764). Nup62-Flag-EPEA displayed an increased mass shift in the presence of nEPEA-OGT(13) relative to the control samples. Transfection of Nup62-Flag-EPEA with the catalytically attenuated nEPEA-OGT(13)^{H498A} produced a smaller mass-shift relative to the control, which indicated specificity in the mass shift due to alteration of the O-GlcNAc occupancy. The HA-nEPEA-OGT(4) mass-shifted NUP62-Flag-EPEA to a similar degree as nEPEAOGT(13), while complete removal of the TPR repeat domain in nEPEA-OGT(0) produced negligible shifts in O-GlcNAc stoichiometry to NUP62-EPEA relative to the control. HA-nEPEA-OGT constructs with three, two, and one TPRs were evaluated to delineate a point at which glycosyltransferase activity on the target protein was lost. Although glycosyltransferase activity decreased with three TPRs or fewer, HA-nEPEA-OGT(1) produced detectable increases in glycosylation of the target protein Nup62-Flag-EPEA. Similarly, under proximity-directed conditions with the nanobody, only the single TPR in the HA-nEPEA-OGT(1) was needed to produce detectable increases in O-GlcNAcylation of c-JUN-Flag-EPEA by the glycoprotein enrichment assay and mass shift assay.

The interaction between HA-nEPEA-OGT(13) and Nup62-Flag-EPEA was confirmed by coimmunoprecipitation. Immunoprecipitation for OGT or for Nup62 showed a greater association with HA-nEPEA-OGT and not OGT alone. The ability of different nanobody fusions to transfer O-GlcNAc to the EPEA tag on c-JUN-Flag-EPEA was evaluated to characterize specificity of the proximity-directed OGT constructs for the target protein. The degree of OGlcNAc was determined by the glycoprotein quantification assay following transfection in GFP-expressing HEK293T cells. HA-nEPEA-OGT(13) was found to increase O-GlcNAc occupancy on c-JUN-Flag-EPEA relative to samples co-expressed with HA-nGFP-OGT(13). In contrast, O-GlcNAc levels on c-JUN-Flag-EPEA co-expressed with HA-nGFP-OGT(4) were limited. However, introduction of the matched nanobody that recognizes the EPEA tag, HA-nEPEA-OGT(4), successfully restored the O-GlcNAc levels on c-JUN-Flag-EPEA. Thus, the evaluated nanobody-OGT fusion proteins were able to selectively redirect OGT to introduce O-GlcNAc on the target substrate and HA-nEPEA-OGT(4) fusion protein displayed the highest selectively and glycosyltransferase activity.

HA-nEPEA-OGT(13) and HA-nEPEA-OGT(4) were evaluated for the ability to site selectively introduce O-GlcNAc to a broader set of protein targets. In an analogous experiment, JunB-Flag-EPEA and TET3(680)-Flag-EPEA were co-expressed with HA-nEPEA-OGT(13) or HA-nEPEA-OGT(4) for affinity purification, digestion, and analysis by mass spectrometry. Of the four glycopeptides identified from JunB, three glycopeptides were identified in all samples including the control. Two glycosites were confidently assigned between the control and at least one nanobody-OGT sample and the additional glycosites and glycopeptides were observed from JunB-Flag-EPEA co-expressed with HA-nEPEA-OGT(4). In line with the elevated glycosylation of the target protein by proximity-directed HA-nEPEA-OGT(4), analysis of TET3(680)-Flag-EPEA revealed four regions of glycosylation from HA-nEPEA-OGT(4) and three major glycopeptides from HA-nEPEA-OGT(13), and two glycosites confidently localized to T966 and S969. While these were the first glycosites identified on human TET3, these regions of glycosylation approximately aligned with previous glycopeptide identifications from mouse TET3 (Bauer, C; Göbel, K.; Nagaraj, N.; Colantuoni, C.; Wang, M.; Muller, U.; Kremmer, E.; Rottach, A.; Leonhardt, H. Journal of Biological Chemistry 2015, 290, 4801).

### Example 7: Targeting Endogenous Proteins for O-GlcNAcylation with Proximity-Directed OGT

Because the EPEA nanobody was developed against α-synuclein, a mass-shift assay was used to determine if the nanobody-OGT nEPEA(4) could increase glycosylation of endogenous α-synuclein in a selective manner. This mass-shift assay used a chemical reporter for O-GlcNAc to install a PEG-5 kDa reporter molecule for determination of OGlcNAc protein occupancy (FIG. 6A). (Rexach, J. E. et al. Quantification of O-glycosylation stoichiometry and dynamics using resolvable mass tags. Nat Chem Biol 6, 645-651 (2010).) An increase in O-GlcNAc levels on α-synuclein was observed by a mass shift assay with expression of RFP(13) and nEPEA(4) but not the catalytically inactive mutant nEPEA(4,K852A) (FIG. 6B). Expression of the untargeted RFP(13) resulted in a dramatic increase in O-GlcNAcylation across the global HEK293T cell proteome that was substantially reduced in the nEPEA(4) sample. A comparison of the glycosylated α-synuclein to the global levels of O-GlcNAc observed allowed the assigning of a selectivity factor. This selectivity factor represented an increase in the glycosylation of α-synuclein with reduced perturbations to the global O-GlcNAc levels observed. Taken together, these data suggest the high selectivity and versatility of proximity-directed nanobody-OGT(4) constructs to transfer O-GlcNAc to their intended target protein, either as a tagged or endogenous target protein, with reduced impact on global O-GlcNAc levels as compared to the current benchmark of overexpressing full-length OGT.

### Example 7: Comparison of the nEPEA-OGT(13) and nEPEA-OGT(4) Constructs

HA-nEPEA-OGT(13) and HA-nEPEA-OGT(4) constructs were compared. One described function for O-GlcNAc is the ability of OGT association with ten-eleven translocation 3 (TET3) to result in increased O-GlcNAc modification and alteration to TET3 subcellular localization (Zhang, Q.; Liu, X.; Gao, W.; Li, P.; Hou, J.; Li, J.; Wong, J. Journal of Biological Chemistry, 2014, 289, 5986). Overexpression of OGT or upregulation of glucose metabolism caused TET3 localization to shift from the nucleus to the cytoplasm, thus negatively regulating TET3 activity on DNA (Zhang, Q.; Liu, X.; Gao, W.; Li, P.; Hou, J.; Li, J.; Wong, J. Journal of Biological Chemistry, 2014, 289, 5986). However, these methods produced global shifts in O-GlcNAc levels and specific O-GlcNAc sites on TET3 that drive the shift in subcellular localization were not identified. Thus, proximity-directed HA-nEPEA-OGT to TET3-EPEA was applied to determine if the direct interaction between HA-nEPEA-OGT and TET3-EPEA would cause a similar shift in subcellular localization.

Immunofluorescence imaging of TET3-Flag-EPEA expressed in HEK293T cells revealed a subcellular localization primarily within the nucleus. Co-expression of TET3-Flag-EPEA with HA-nEPEA-OGT(13) produced a distinct transition of TET3-Flag-EPEA from the nucleus to the cytoplasm. However, co-expression of TET3-Flag-EPEA with HA-nEPEA-OGT(4) was found distinctly in the nucleus, despite the observed catalytic activity on a number of substrate proteins, including TET3(680)-Flag-EPEA. This pointed to a distinct role for the TPR domain in shifting TET3-Flag-EPEA subcellular localization. The requirement for associations through the TPR domain in shifting TET3 was confirmed by the catalytically attenuated HA-nEPEA-OGT(13)^{H498A}, which exhibited reduced rates of glycosyltransferase activity (Zhang, Q.; Liu, X.; Gao, W.; Li, P.; Hou, J.; Li, J.; Wong, J. Journal of Biological Chemistry, 2014, 289, 5986), and catalytically-dead HA-nEPEA-OGT(13)^{K852A}, which cannot bind to UDP-GlcNAc (Martines-Fleites, C.; Macauley, M. S.; He, Y.; He, Y.; Shen, D. L.; Vocadlo, D. J.; Davies, G. J. Nature Structural & Molecular Biology 2008, 15, 764). Subcellular localization of TET3-Flag-EPEA co-transfected with both of constructs displayed clear subcellular localization in the cytoplasm. A similar shift in cytoplasmic localization of TET3(680)-Flag-EPEA was observed with the HA-nEPEA-OGT(13), but not with the two glycosyltransferase deficient mutants or HA-nEPEA-OGT(4), pointing to contributions from both the scaffolding of the TPR domain and glycosyltransferase activity resulting in TET3 translocation.

The O-GlcNAc stoichiometry on TET3(680)-Flag-EPEA was increased in the presence of both HA-nEPEA-OGT(13) and HA-nEPEA-OGT(4). There was no increase in O-GlcNAc stoichiometry observed for the mutant proteins or the control lanes. The discrepancy between the subcellular localization of TET3 expressed with HA-nEPEA-OGT(13) and HA-nEPEA-OGT(4) indicated that TET3 subcellular localization was not dependent on O-GlcNAc stoichiometry alone. The data pointed to the dependence of TET3 cytoplasmic localization specifically on scaffolding associations with the 13-4 TPR domain, which may occurred in response to elevated full-length OGT expression. Both HA-nEPEA-OGT(13) and HA-nEPEAOGT(4) induced protein-specific O-GlcNAc to a series of target proteins and were used to differentiate the scaffolding and enzymatic functions of OGT, as illustrated by the differential changes to TET3 subcellular localization.

### Example 8: Alpha-Synuclein as the Target Protein

HEK293T cells were transfected with pcDNA plasmid (control) or nEPEA-OGT(4.5) for 48 h prior to immunofluorescence. Cells were fixed with 4% paraformaldehyde for 15 min, permeabilized by 0.1% Triton-X in PBS for 20 min, and blocked with 3% BSA/TBST for at least 1 h. Subsequently, cells were incubated with the primary antibodies overnight at 4° C., followed by the secondary antibodies for 1 h, stained the nucleus with DAPI for 10 min. The coverslip was finally mounted in an anti-fade reagent for confocal fluorescence microscopy.

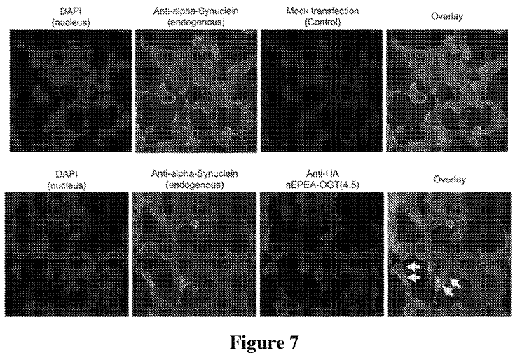

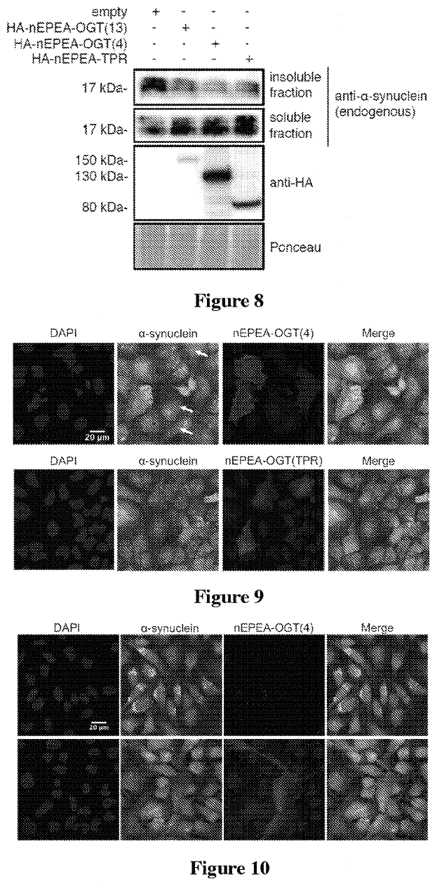

Cells co-transfected with nEPEA-OGT(4.5) to target alpha-synuclein (a-Syn) proteins tend to have less endogenous a-Syn aggregates (FIG. 7). The arrows in FIG. 7 show two contrasting cells with and without nanobody-OGT expression. Using analogues procedures as described above, FIGS. 8-10 show the ability of fusion proteins to act on alpha-synuclein. Western blot analysis of alpha-synuclein with or without expression of HA-nEPEA-OGT(13), HA-nEPEA-OGT(4), or the TPR domain alone (HA-nEPEA-TPR) showed alpha-synuclein aggregates were separated into the insoluble fraction and non-aggregated alpha-synuclein was separated into the soluble fraction (FIG. 8). FIGS. 9 and 10 show α-synuclein aggregates in U2OS and HeLa cells, respectively with or without HA-nEPEA-OGT(4), thus demonstrating the ability of these fusion proteins to act on α-synuclein.

### REFERENCES

1. Lund, P. J., Elias, J. E. & Davis, M. M. Global Analysis of O-GlcNAc Glycoproteins in Activated Human T Cells. J Immunol 197, 3086-3098 (2016).
2. Yi, W. et al. Phosphofructokinase 1 Glycosylation Regulates Cell Growth and Metabolism Science 337, 975-980 (2012).
3. Yuzwa, S. A. et al. Increasing O-GlcNAc slows neurodegeneration and stabilizes tau against aggregation. Nat Chem Biol 8, 393-399 (2012).
4. Lagerlof, O. et al. The nutrient sensor OGT in PVN neurons regulates feeding. Science 351, 1293-1296 (2016).
5. Gambetta, M. C. & Muller, J. A critical perspective of the diverse roles of O-GlcNAc transferase in chromatin. Chromosoma 124, 429-442 (2015).
6. Gloster, T. M. et al. Hijacking a biosynthetic pathway yields a glycosyltransferase inhibitor within cells. Nat Chem Biol 7, 174-181 (2011); Martin, S. E. S. et al. Structure-Based Evolution of Low Nanomolar O-GlcNAc Transferase Inhibitors. J Am Chem Soc 140, 13542-13545 (2018).
7. Leney, A. C. et al. Elucidating crosstalk mechanisms between phosphorylation and OGlcNAcylation. Proc Natl Acad Sci 114, E7255-E7261 (2017).
8. Zhu, Y. et al. O-GlcNAc occurs cotranslationally to stabilize nascent polypeptide chains. Nat Chem Biol 11, 319-325 (2015).
9. Trinidad, J. C. et al. Global identification and characterization of both O-GlcNAcylation and phosphorylation at the murine synapse. Mol Cell Proteomics 11, 215-229 (2012); Hahne, H. et al. Proteome wide purification and identification of O-GlcNAc-modified proteins using click chemistry and mass spectrometry. J Proteome Res 12, 927-936 (2013); Wang, X. et al. A novel quantitative mass spectrometry platform for determining sitespecific protein O-GlcNAcylation dynamics. Mol Cell Proteomics (2016); Wang, S. et al. Quantitative proteomics identifies altered O-GlcNAcylation of structural, synaptic and memory-associated proteins in Alzheimer's disease. J Pathol 243, 78-88 (2017).
10. Woo, C. M. et al. Mapping and Quantification of Over 2000 O-linked Glycopeptides in Activated Human T Cells with Isotope-Targeted Glycoproteomics (Isotag). Mol Cell Proteomics 17, 764-775 (2018).
11. Shafi, R. et al. The O-GlcNAc transferase gene resides on the X chromosome and is essential for embryonic stem cell viability and mouse ontogeny. Proc Natl Acad Sci 97, 5735-5739 (2000).
12. Yang, Y. R. et al. O-GlcNAcase is essential for embryonic development and maintenance of genomic stability. Aging Cell 11, 439-448 (2012).
13. O'Donnell, N., Zachara, N. E., Hart, G. W. & Marth, J. D. Ogt-dependent X chromosomelinked protein glycosylation is a requisite modification in somatic cell function and embryo viability. Mol Cell Biol 24, 1680-1690 (2004).
14. Haltiwanger, R. S., Blomberg, M. A. & Hart, G. W. Glycosylation of nuclear and cytoplasmic proteins. Purification and characterization of a uridine diphospho Nacetylglucosamine: polypeptide beta-N-acetylglucosaminyltransferase. J Biol Chem 267, 9005-9013 (1992).
15. Lazarus, M. B. et al. Structure of human O-GlcNAc transferase and its complex with a peptide substrate. Nature 469, 564-567 (2011).
16. Iyer, S. P. N. & Hart, G. W. Roles of the Tetratricopeptide Repeat Domain in O-GlcNAc Transferase Targeting and Protein Substrate Specificity. J Biol Chem 278, 24608-24616 (2003).
17. Pathak, S. et al. The active site of O-GlcNAc transferase imposes constraints on substrate sequence. Nat Struct Mol Biol 22, 744-750 (2015).
18. Stanton, B. Z., Chory, E. J. & Crabtree, G. R. Chemically induced proximity in biology and medicine. Science 359 (2018).
19. Kirchhofer, A. et al. Modulation of protein properties in living cells using nanobodies. Nat Struct Mol Biol 17, 133-138 (2010); Anton, T. & Bultmann, S. Site-specific recruitment of epigenetic factors with a modular CRISPR/Cas system. Nucleus 8, 279-286 (2017).
20. Caussinus, E., Kanca, O. & Affolter, M. Fluorescent fusion protein knockout mediated by anti-GFP nanobody. Nat Struct Mol Biol 19, 117-121 (2012).
21. Dmitriev, O. Y., Lutsenko, S. & Muyldermans, S. Nanobodies as Probes for Protein Dynamics in Vitro and in Cells. J Biol Chem 291, 3767-3775 (2016).
22. Kubala, M. H., Kovtun, O., Alexandrov, K. & Collins, B. M. Structural and thermodynamic analysis of the GFP:GFP-nanobody complex. Protein Sci 19, 2389-2401 (2010).
23. De Genst, E. J. et al. Structure and properties of a complex of alpha-synuclein and a singledomain camelid antibody. J Mol Biol 402, 326-343 (2010).
24. Lee, B. R. & Kamitani, T. Improved Immunodetection of Endogenous α-Synuclein. PLoS ONE 6, e23939 (2011).
25. Rexach, J. E. et al. Quantification of O-glycosylation stoichiometry and dynamics using resolvable mass tags. Nat Chem Biol 6, 645-651 (2010).
26. Lubas, W. A. & Hanover, J. A. Functional Expression of O-linked GlcNAc Transferase: DOMAIN STRUCTURE AND SUBSTRATE SPECIFICITY. J Biol Chem 275, 10983-10988 (2000).
27. Thompson, J. W., Griffin, M. E. & Hsieh-Wilson, L. C. in Meth Enzymol Vol. 598 (ed Barbara Imperiali) 101-135 (Academic Press, 2018).
28. Woo, C. M. et al. Isotope-targeted glycoproteomics (IsoTaG): a mass-independent platform for intact N- and O-glycopeptide discovery and analysis. Nat Meth 12, 561-567 (2015).
29. Cheng, X. & Hart, G. W. Glycosylation of the murine estrogen receptor-α. J Steroid Biochem 75, 147-158 (2000).
30. Marotta, N. P. et al. O-GlcNAc modification blocks the aggregation and toxicity of the protein α-synuclein associated with Parkinson's disease. Nat Chem 7, 913-920 (2015).
31. Spencer, D. M., Wandless, T. J., Schreiber, S. L. & Crabtree, G. R. Controlling signal transduction with synthetic ligands. Science 262, 1019-1024 (1993); Fegan, A., White, B., Carlson, J. C. T. & Wagner, C. R. Chemically Controlled Protein Assembly: Techniques and Applications. Chem Rev 110, 3315-3336 (2010).
32. Banaszynski, L. A. et al. A Rapid, Reversible, and Tunable Method to Regulate Protein Function in Living Cells Using Synthetic Small Molecules. Cell 126, 995-1004 (2006).
33. Moriya, H. Quantitative nature of overexpression experiments. Mol Biol Cell 26, 3932 3939 (2015).
34. Lira-Navarrete, E. et al. Dynamic interplay between catalytic and lectin domains of GalNActransferases modulates protein O-glycosylation. Nat Commun 6, 6937 (2015).
35. Darabedian, N. et al. Optimization of chemoenzymatic mass-tagging by strain-promoted cycloaddition (SPAAC) for the determination of O-GlcNAc stoichiometry by Western blotting. Biochemistry (2018).

SEQUENCES
HA-nEPEA-OGT(4) (pcDNA3.1-HA-nEPEA- [symbol] -OGT(4))
nucleotide sequence
(SEQ ID NO: 35)
GACGGATCGGGAGATCTCCCGATCCCCTATGGTGCACTCTCAGTACAATCTGCTCTGATGCC
GCATAGTTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTGCTTAGGG
TTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTAT
ATAAGCAGAGCTCTCTGGCTAACTAGAGAACCCACTGCTTACTGGCTTATCGAAATTAATAC
GACTCACTATAGGGAGACCCAAGCTGGCGAGCGTTTAAGCTTGAGCAATGGCATACCCATAC
GATGTTCCAGATTACGCTGCGATCGCAATGGGCCAGCTGGTGGAGAGCGGCGGCGGCAGCGT
GCAGGCCGGCGGCAGCCTGAGGCTGAGCTGCGCCGCCAGCGGCATCGACAGCAGCAGCTACT
GCATGGGCTGGTTCAGGCAGAGGCCCGGCAAGGAGAGGGAGGGCGTGGCCAGGATCAACGGC
CTGGGCGGCGTGAAGACCGCCTACGCCGACAGCGTGAAGGACAGGTTCACCATCAGCAGGGA
CAACGCCGAGAACACCGTGTACCTGCAGATGAACAGCCTGAAGCCCGAGGACACCGCCATCT
ACTACTGCGCCGCCAAGTTCAGCCCCGGCTACTGCGGCGGCAGCTGGAGCAACTTCGGCTAC
TGGGGCCAGGGCACCCAGGTTACTGTGAGCTCTGGCGCGCCA

[symbol]

[symbol]

[symbol]

[symbol]

[symbol]

[symbol]

GGATCCATGGCAGACTCTTTGA
ATAACCTTGCCAACATCAAACGGGAACAGGGCAACATTGAAGAGGCAGTTCGCCTGTATCGC
AAAGCATTAGAAGTCTTCCCAGAGTTTGCTGCTGCACATTCCAATTTAGCAAGTGTACTGCA
ACAGCAGGGCAAGCTGCAGGAAGCACTGATGCACTATAAAGAAGCCATACGAATTAGTCCTA
CATTTGCTGATGCTTATTCCAATATGGGAAACACTCTAAAGGAGATGCAGGATGTGCAGGGC
GCTTTGCAGTGTTATACTCGTGCCATCCAGATTAATCCTGCCTTTGCTGATGCACACAGCAA
TCTGGCCTCCATTCACAAGGATTCAGGGAATATCCCAGAAGCAATAGCTTCTTACCGCACAG
CTCTGAAACTTAAGCCTGACTTTCCTGATGCTTATTGTAACTTGGCTCATTGCCTACAGATT
GTCTGTGATTGGACAGACTATGATGAGCGGATGAAGAAATTGGTTAGTATTGTAGCTGAGCA
GCTAGAGAAGAATAGACTGCCTTCTGTCCATCCTCACCATAGCATGCTGTACCCTCTTTCCC
ATGGCTTCAGGAAGGCTATTGCAGAGAGGCATGGGAATCTCTGCTTGGATAAGATTAATGTC
CTTCATAAACCACCATATGAACATCCAAAAGACTTGAAGCTCAGTGATGGCCGATTGCGTGT
AGGCTATGTGAGTTCTGACTTCGGGAATCACCCTACTTCACACCTTATGCAGTCTATTCCAG
GCATGCATAATCCTGATAAGTTTGAGGTATTCTGCTATGCCTTGAGCCCGGATGATGGTACA
AACTTTCGAGTGAAGGTGATGGCGGAAGCCAATCATTTCATTGATCTTTCTCAGATTCCTTG
TAATGGAAAAGCAGCCGACCGCATCCACCAAGATGGAATTCACATCCTTGTGAATATGAATG
GGTATACCAAGGGTGCTCGGAATGAGCTCTTTGCTCTTAGGCCAGCTCCTATTCAGGCCATG
TGGCTGGGCTACCCTGGGACTAGTGGTGCACTGTTCATGGATTACATCATCACTGATCAGGA
AACTTCCCCAGCTGAAGTTGCAGAGCAGTATTCTGAGAAACTGGCTTATATGCCCCATACTT
TCTTTATTGGTGATCATGCTAATATGTTCCCTCACCTGAAGAAAAAAGCAGTCATCGATTTT
AAATCCAATGGGCACATTTATGATAATCGGATAGTTCTGAATGGCATCGATCTCAAAGCATT
TCTCGATAGCCTACCCGATGTGAAGATTGTCAAGATGAAATGTCCTGATGGAGGTGACAATC
CAGACAGCAGTAACACAGCTCTTAATATGCCCGTTATTCCCATGAATACGATTGCAGAAGCA
GTAATTGAAATGATTAACAGAGGGCAGATTCAGATAACAATTAACGGATTCAGTATTAGCAA
TGGACTGGCGACTACACAGATTAATAATAAGGCTGCAACCGGAGAGGAAGTTCCCCGTACCA
TTATTGTAACCACCCGTTCCCAGTATGGGCTACCAGAAGATGCCATTGTGTACTGTAACTTT
AATCAGTTATATAAAATTGACCCATCTACCCTGCAGATGTGGGCAAATATTCTGAAACGTGT
GCCTAACAGCGTGCTTTGGCTGTTGCGTTTTCCAGCAGTAGGAGAACCCAATATTCAACAAT
ATGCACAAAATATGGGCCTTCCCCAGAACCGTATCATTTTCTCACCTGTGGCTCCTAAAGAG
GAGCATGTCAGGAGAGGTCAGCTGGCTGATGTCTGCCTGGATACTCCTTTGTGTAATGGACA
CACCACAGGGATGGATGTTCTCTGGGCAGGAACACCCATGGTGACTATGCCAGGAGAGACTC
TTGCCTCTCGAGTTGCAGCTTCTCAGCTTACTTGTCTAGGATGTCTCGAGCTCATTGCTAAA
AGCAGACAGGAATATGAAGACATAGCTGTGAAACTGGGAACCGATCTAGAATACCTGAAGAA
AATTCGTGGCAAAGTCTGGAAACAGAGAATATCTAGCCCTCTGTTCAACACCAAACAATACA
CAATGGAATTAGAGCGACTTTATCTGCAGATGTGGGAGCATTATGCAGCTGGCAACAAACCT
GACCACATGATTAAGCCTGTTGAAGTCACCGAGTCAGCCTAAGCGGCCGCTCGAGTCTAGAG
GGCCCGTTTAAACCCGCTGATCAGCCGACTGTGCCTTCTAGTTGCCAGCCATCTGTTGTTTG
CCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGG
CAGGACAGCAAGGGGGAGGATTGGGAAGACAATAGCAGGCATGCTGGGGATGCGGTGGGCTC
TATGGCTTCTGAGGCGGAAAGAACCAGCTGGGGCTCTAGGGGGTATCCCCACGCGCCCTGTA
GCGGCGCATTAAGCGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGTTCGCCGGCTTTCC
CCGTCAAGCTCTAAATCGGGGGCTCCCTTTAGGGTTCCGATTTAGTGCTTTACGGCACCTCG
ACCCCAAAAAACTTGATTAGGGTGATGGTTCACGTAGTGGGCCATCGCCCTGATAGACGGTT
TTTCGCCCTTTGACGTTGGAGTCCACGTTCTTTAATAGTGGACTCTTGTTCCAAACTGGAAC
AACACTCAACCCTATCTCGGTCTATTCTTTTGATTTATAAGGGATTTTGCCGATTTCGGCCT
ATTGGTTAAAAAATGAGCTGATTTAACAAAAATTTAACGCGAATTAATTCTGTGGAATGTGT
GTCAGTTAGGGTGTGGAAAGTCCCCAGGCTCCCCAGCAGGCAGAAGTATGCAAAGCATGCAT
CTCAATTAGTCAGCAACCAGGTGTGGAAAGTCCCCAGGCTCCCCAGCAGGCAGAAGTATGCA
AAGCATGCATCTCAATTAGTCAGCAACCATAGTCCCGCCCCTAACTCCGCCCATCCCGCCCC
TAACTCCGCCCAGTTCCGCCCATTCTCCGCCCCATGGCTGACTAATTTTTTTTATTTATGCA
GAGGCCGAGGCCGCCTCTGCCTCTGAGCTATTCCAGAAGTAGTGAGGAGGCTTTTTTGGAGG
CCTAGGCTTTTGCAAAAAGCTCCCGGGAGCTTGTATATCCATTTTCGGATCTGATCAAGAGA
CAGGATGAGGATCGTTTCGCATGATTGAACAAGATGGATTGCACGCAGGTTCTCCGGCCGCT
TGGGTGGAGAGGCTATTCGGCTATGACTGGGCACAACAGACAATCGGCTGCTCTGATGCCGC
CGTGTTCCGGCTGTCAGCGCAGGGGCGCCCGGTTCTTTTTGTCAAGACCGACCTGTCCGGTG
CCCTGAATGAACTGCAGGACGAGGCAGCGCGGCTATCGTGGCTGGCCACGACGGGCGTTCCT
TGCGCAGCTGTGCTCGACGTTGTCACTGAAGCGGGAAGGGACTGGCTGCTATTGGGCGAAGT
GCCGGGGCAGGATCTCCTGTCATCTCACCTTGCTCCTGCCGAGAAAGTATCCATCATGGCTG
ATGCAATGCGGCGGCTGCATACGCTTGATCCGGCTACCTGCCCATTCGACCACCAAGCGAAA
CATCGCATCGAGCGAGCACGTACTCGGATGGAAGCCGGTCTTGTCGATCAGGATGATCTGGA
CGAAGAGCATCAGGGGCTCGCGCCAGCCGAACTGTTCGCCAGGCTCAAGGCGCGCATGCCCG
ACGGCGAGGATCTCGTCGTGACCCATGGCGATGCCTGCTTGCCGAATATCATGGTGGAAAAT
GGCCGCTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTTCCTCG
TGCTTTACGGTATCGCCGCTCCCGATTCGCAGCGCATCGCCTTCTATCGCCTTCTTGACGAG
TTCTTCTGAGCGGGACTCTGGGGTTCGAAATGACCGACCAAGCGACGCCCAACCTGCCATCA
CGAGATTTCGATTCCACCGCCGCCTTCTATGAAAGGTTGGGCTTCGGAATCGTTTTCCGGGA
CGCCGGCTGGATGATCCTCCAGCGCGGGGATCTCATGCTGGAGTTCTTCGCCCACCCCAACT
TGTTTATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTTATCATGT
CTGTATACCGTCGACCTCTAGCTAGAGCTTGGCGTAATCATGGTCATAGCTGTTTCCTGTGT
GAAATTGTTATCCGCTCACAATTCCACACAACATACGAGCCGGAAGCATAAAGTGTAAAGCC
TGGGGTGCCTAATGAGTGAGCTAACTCACATTAATTGCGTTGCGCTCACTGCCCGCTTTCCA
GTCGGGAAACCTGTCGTGCCAGCTGCATTAATGAATCGGCCAACGCGCGGGGAGAGGCGGTT
TGCGTATTGGGCGCTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGC
TCCAAGCTGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTACCTTCGG
AAAAAGAGTTGGTAGCTCTTGATCCGGCAAACAAACCACCGCTGGTAGCGGTTTTTTTGTTT
GCAAGCAGCAGATTACGCGCAGAAAAAAAGGATCTCAAGAAGATCCTTTGATCTTTTCTACG
GGGTCTGACGCTCAGTGGAACGAAAACTCACGTTAAGGGATTTTGGTCATGAGATTATCAAA
AAGGATCTTCACCTAGATCCTTTTAAATTAAAAATGAAGTTTTAAATCAATCTAAAGTATAT
ATGAGTAAACTTGGTCTGACAGTTACCAATGCTTAATCAGTGAGGCACCTATCTCAGCGATC
TGTCTATTTCGTTCATCCATAGTTGCCTGACTCCCCGTCGTGTAGATAACTACGATACGGGA
GGGCTTACCATCTGGCCCCAGTGCTGCAATGATACCGCGAGACCCACGCTCACCGGCTCCAG
ATTTATCAGCAATAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAGTTCGCCAGTTAA
TAGTTTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTTGGCCGCAGTGTT
ATCACTCATGGTTATGGCAGCACTGCATAATTCTCTTACTGTCATGCCATCCGTAAGATGCT
TTTCTGTGACTGGTGAGTACTCAACCAAGTCATTCTGAGAATAGTGTATGCGGCGACCGAGT
TGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTT
TCTGGGTGAGCAAAAACAGGAAGGCAAAATGCCGCAAAAAAGGGAATAAGGGCGACACGGAA
ATGTTGAATACTCATACTCTTCCTTTTTCAATATTATTGAAGCATTTATCAGGGTTATTGTC
TCATGAGCGGATACATATTTGAATGTATTTAGAAAAATAAACAAATAGGGGTTCCGCGCACA
TTTCCCCGAAAAGTGCCACCTGACGTC
HA-nEPEA-OGT(4) (pcDNA3.1-HA-nEPEA- [symbol] -OGT(4))
protein sequence
(SEQ ID NO: 36)
MAYPYDVPDYAAIAMGQLVESGGGSVQAGGSLRLSCAASGIDSSSYCMGWFRQRPGKEREGV
ARINGLGGVKTAYADSVKDRFTISRDNAENTVYLQMNSLKPEDTAIYYCAAKFSPGYCGGSW
SNFGYWGQGTQVTVSSGAP [symbol] [symbol] GSMADSLNNLANIKRE
QGNIEEAVRLYRKALEVFPEFAAAHSNLASVLQQQGKLQEALMHYKEAIRISPTFADAYSNM
GNTLKEMQDVQGALQCYTRAIQINPAFADAHSNLASIHKDSGNIPEAIASYRTALKLKPDFP
DAYCNLAHCLQIVCDWTDYDERMKKLVSIVAEQLEKNRLPSVHPHHSMLYPLSHGFRKAIAER
HGNLCLDKINVLHKPPYEHPKDLKLSDGRLRVGYVSSDFGNHPTSHLMQSIPGMHNPDKFEVF
CYALSPDDGTNFRVKVMAEANHFIDLSQIPCNGKAADRIHQDGIHILVNMNGYTKGARNELFA
LRPAPIQAMWLGYPGTSGALFMDYIITDQETSPAEVAEQYSEKLAYMPHTFFIGDHANMFPHL
AKKKVIDFKSNGHIYDNRIVLNGIDLKAFLDSLPDVKIVKMKCPDGGDNPDSSNTALNMPVIP
MNTIAEAVIEMINRGQIQITINGFSISNGLATTQINNKAATGEEVPRTIIVTTRSQYGLPEDA
IVYCNFNQLYKIDPSTLQMWANILKRVPNSVLWLLRFPAVGEPNIQQYAQNMGLPQNRIIFSP
VAPKEEHVRRGQLADVCLDTPLCNGHTTGMDVLWAGTPMVTMPGETLASRVAASQLTCLGCL
ELIAKSRQEYEDIAVKLGTDLEYLKKIRGKVWKQRISSPLFNTKQYTMELERLYLQMWEHYA
AGNKPDHMIKPVEVTESA
HA-nEPEA-OGT(13) (pcDNA3.1-HA-nEPEA- [symbol] -OGT(13))
Nucleotide sequence
(SEQ ID NO: 37)
GACGGATCGGGAGATCTCCCGATCCCCTATGGTGCACTCTCAGTACAATCTGCTCTGATGCC
GCATAGTTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTGCTTAGGG
TTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTAT
ATAAGCAGAGCTCTCTGGCTAACTAGAGAACCCACTGCTTACTGGCTTATCGAAATTAATAC
GACTCACTATAGGGAGACCCAAGCTGGCGAGCGTTTAAGCTTGAGCAATGGCATACCCATAC
GATGTTCCAGATTACGCTGCGATCGCAATGGGCCAGCTGGTGGAGAGCGGCGGCGGCAGCGT
GCAGGCCGGCGGCAGCCTGAGGCTGAGCTGCGCCGCCAGCGGCATCGACAGCAGCAGCTACT
GCATGGGCTGGTTCAGGCAGAGGCCCGGCAAGGAGAGGGAGGGCGTGGCCAGGATCAACGGC
CTGGGCGGCGTGAAGACCGCCTACGCCGACAGCGTGAAGGACAGGTTCACCATCAGCAGGGA
CAACGCCGAGAACACCGTGTACCTGCAGATGAACAGCCTGAAGCCCGAGGACACCGCCATCT
ACTACTGCGCCGCCAAGTTCAGCCCCGGCTACTGCGGCGGCAGCTGGAGCAACTTCGGCTAC
TGGGGCCAGGGCACCCAGGTTACTGTGAGCTCTGGCGCGCCA

[symbol]

[symbol]

[symbol]

[symbol]

[symbol]

[symbol]

GGATCCATGGCGTCTTCCGTGG
GCAACGTGGCCGACAGTACAGAACCAACGAAACGTATGCTTTCCTTCCAAGGGTTAGCTGAG
TTGGCACATCGAGAATATCAGGCAGGAGATTTTGAGGCAGCTGAGAGACACTGCATGCAGCT
CTGGAGACAAGAGCCTGACAATACTGGTGTTCTTTTATTACTTTCATCTATACACTTCCAGT
GTCGAAGGCTGGACAGATCTGCTCATTTTAGCACCTTGGCAATTAAACAGAATCCCCTTCTA
GCAGAAGCCTATTCGAATTTAGGAAATGTGTACAAGGAAAGAGGGCAGTTGCAGGAAGCAAT
CGAGCATTATCGACATGCCTTGCGGCTGAAGCCTGATTTCATTGATGGTTATATTAACCTGG
CAGCAGCCTTGGTAGCAGCAGGTGACATGGAAGGAGCAGTACAAGCCTATGTCTCTGCTCTT
CAGTACAATCCTGATTTGTACTGTGTTCGCAGTGACCTGGGGAACCTGCTCAAAGCCCTGGG
TCGCTTGGAAGAAGCCAAGGCATGTTATTTGAAAGCAATTGAGACGCAACCAAACTTTGCAG
TAGCCTGGAGTAATCTCGGCTGTGTTTTCAATGCACAAGGGGAGATTTGGCTGGCTATTCAT
CACTTTGAAAAGGCTGTCACCCTTGACCCAAATTTTCTGGATGCTTATATCAATTTAGGAAA
TGTCTTGAAAGAGGCACGCATTTTTGACAGAGCTGTCGCAGCTTATCTTCGTGCCTTAAGTT
TGAGCCCAAATCATGCGGTGGTGCACGGCAACCTGGCTTGTGTGTACTACGAGCAAGGCCTA
ATAGACCTGGCCATTGATACCTACAGGAGAGCTATCGAACTGCAACCCCATTTCCCCGATGC
TTACTGCAACCTAGCAAATGCTCTCAAAGAGAAGGGCAGTGTTGCTGAAGCAGAAGACTGTT
ATAACACAGCTCTTCGTCTGTGTCCTACTCATGCAGACTCTTTGAATAACCTTGCCAACATC
AAACGGGAACAGGGCAACATTGAAGAGGCAGTTCGCCTGTATCGCAAAGCATTAGAAGTCTT
CCCAGAGTTTGCTGCTGCACATTCCAATTTAGCAAGTGTACTGCAACAGCAGGGCAAGCTGC
AGGAAGCACTGATGCACTATAAAGAAGCCATACGAATTAGTCCTACATTTGCTGATGCTTAT
TCCAATATGGGAAACACTCTAAAGGAGATGCAGGATGTGCAGGGCGCTTTGCAGTGTTATAC
TCGTGCCATCCAGATTAATCCTGCCTTTGCTGATGCACACAGCAATCTGGCCTCCATTCACA
AGGATTCAGGGAATATCCCAGAAGCAATAGCTTCTTACCGCACAGCTCTGAAACTTAAGCCT
GACTTTCCTGATGCTTATTGTAACTTGGCTCATTGCCTACAGATTGTCTGTGATTGGACAGA
CTATGATGAGCGGATGAAGAAATTGGTTAGTATTGTAGCTGAGCAGCTAGAGAAGAATAGAC
TGCCTTCTGTCCATCCTCACCATAGCATGCTGTACCCTCTTTCCCATGGCTTCAGGAAGGCT
ATTGCAGAGAGGCATGGGAATCTCTGCTTGGATAAGATTAATGTCCTTCATAAACCACCATA
TGAACATCCAAAAGACTTGAAGCTCAGTGATGGCCGATTGCGTGTAGGCTATGTGAGTTCTG
ACTTCGGGAATCACCCTACTTCACACCTTATGCAGTCTATTCCAGGCATGCATAATCCTGAT
AAGTTTGAGGTATTCTGCTATGCCTTGAGCCCGGATGATGGTACAAACTTTCGAGTGAAGGT
GATGGCGGAAGCCAATCATTTCATTGATCTTTCTCAGATTCCTTGTAATGGAAAAGCAGCCG
ACCGCATCCACCAAGATGGAATTCACATCCTTGTGAATATGAATGGGTATACCAAGGGTGCT
CGGAATGAGCTCTTTGCTCTTAGGCCAGCTCCTATTCAGGCCATGTGGCTGGGCTACCCTGG
GACTAGTGGTGCACTGTTCATGGATTACATCATCACTGATCAGGAAACTTCCCCAGCTGAAG
TTGCAGAGCAGTATTCTGAGAAACTGGCTTATATGCCCCATACTTTCTTTATTGGTGATCAT
GCTAATATGTTCCCTCACCTGAAGAAAAAAGCAGTCATCGATTTTAAATCCAATGGGCACAT
TTATGATAATCGGATAGTTCTGAATGGCATCGATCTCAAAGCATTTCTCGATAGCCTACCCG
ATGTGAAGATTGTCAAGATGAAATGTCCTGATGGAGGTGACAATCCAGACAGCAGTAACACA
GCTCTTAATATGCCCGTTATTCCCATGAATACGATTGCAGAAGCAGTAATTGAAATGATTAA
CAGAGGGCAGATTCAGATAACAATTAACGGATTCAGTATTAGCAATGGACTGGCGACTACAC
AGATTAATAATAAGGCTGCAACCGGAGAGGAAGTTCCCCGTACCATTATTGTAACCACCCGT
TCCCAGTATGGGCTACCAGAAGATGCCATTGTGTACTGTAACTTTAATCAGTTATATAAAAT
TGACCCATCTACCCTGCAGATGTGGGCAAATATTCTGAAACGTGTGCCTAACAGCGTGCTTT
GGCTGTTGCGTTTTCCAGCAGTAGGAGAACCCAATATTCAACAATATGCACAAAATATGGGC
CTTCCCCAGAACCGTATCATTTTCTCACCTGTGGCTCCTAAAGAGGAGCATGTCAGGAGAGG
TCAGCTGGCTGATGTCTGCCTGGATACTCCTTTGTGTAATGGACACACCACAGGGATGGATG
TTCTCTGGGCAGGAACACCCATGGTGACTATGCCAGGAGAGACTCTTGCCTCTCGAGTTGCA
GCTTCTCAGCTTACTTGTCTAGGATGTCTCGAGCTCATTGCTAAAAGCAGACAGGAATATGA
AGACATAGCTGTGAAACTGGGAACCGATCTAGAATACCTGAAGAAAATTCGTGGCAAAGTCT
GGAAACAGAGAATATCTAGCCCTCTGTTCAACACCAAACAATACACAATGGAATTAGAGCGA
CTTTATCTGCAGATGTGGGAGCATTATGCAGCTGGCAACAAACCTGACCACATGATTAAGCC
TGTTGAAGTCACCGAGTCAGCCTAAGCGGCCGCTCGAGTCTAGAGGGCCCGTTTAAACCCGC
TGATCAGCCGACTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAGGGGGA
GGATTGGGAAGACAATAGCAGGCATGCTGGGGATGCGGTGGGCTCTATGGCTTCTGAGGCGG
AAAGAACCAGCTGGGGCTCTAGGGGGTATCCCCACGCGCCCTGTAGCGGCGCATTAAGCGCG
GCGGGTGTGGTGGTTACGCGCAGCGTGACCGCTACACTTGCCAGCGCCCTAGCGCCCGCTCC
TTTCGCTTTCTTCCCTTCCTTTCTCGCCACGTTCGCCGGCTTTCCCCGTCAAGCTCTAAATC
GGGGGCTCCCTTTAGGGTTCCGATTTAGTGCTTTACGGCACCTCGACCCCAAAAAACTTGAT
TAGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATGAG
CTGATTTAACAAAAATTTAACGCGAATTAATTCTGTGGAATGTGTGTCAGTTAGGGTGTGGA
AAGTCCCCAGGCTCCCCAGCAGGCAGAAGTATGCAAAGCATGCATCTCAATTAGTCAGCAAC
CAGGTGTGGAAAGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCCGCCCAGTTCC
GCCCATTCTCCGCCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAGGCTTTTGCAAAA
AGCTCCCGGGAGCTTGTATATCCATTTTCGGATCTGATCAAGAGACAGGATGAGGATCGTTT
CGCATGATTGAACAAGATGGATTGCACGCAGGTTCTCCGGCCGCTTGGGTGGAGAGGCTATT
CGGCTATGACTGGGCACAACAGACAATCGGCTGCTCTGATGCCGCCGTGTTCCGGCTGTCAG
CGCAGGGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGTGCTCGA
CGTTGTCACTGAAGCGGGAAGGGACTGGCTGCTATTGGGCGAAGTGCCGGGGCAGGATCTCC
TGTCATCTCACCTTGCTCCTGCCGAGAAAGTATCCATCATGGCTGATGCAATGCGGCGGCTG
CATACGCTTGATCCGGCTACCTGCCCATTCGACCACCAAGCGAAACATCGCATCGAGCGAGC
ACGTACTCGGATGGAAGCCGGTCTTGTCGATCAGGATGATCTGGACGAAGAGCATCAGGGGC
TCGCGCCAGCCGAACTGTTCGCCAGGCTCAAGGCGCGCATGCCCGACGGCGAGGATCTCGTC
GTGACCCATGGCGATGCCTGCTTGCCGAATATCATGGTGGAAAATGGCCGCTTTTCTGGATT
CATCGACTGTGGCCGGCTGGGTGTGGCGGACCGCTATCAGGACATAGCGTTGGCTACCCGTG
ATATTGCTGAAGAGCTTGGCGGCGAATGGGCTGACCGCTTCCTCGTGCTTTACGGTATCGCC
GCTCCCGATTCGCAGCGCATCGCCTTCTATCGCCTTCTTGACGAGTTCTTCTGAGCGGGACT
CTGGGGTTCGAAATGACCGACCAAGCGACGCCCAACCTGCCATCACGAGATTTCGATTCCAC
CGCCGCCTTCTATGAAAGGTTGGGCTTCGGAATCGTTTTCCGGGACGCCGGCTGGATGATCC
TCCAGCGCGGGGATCTCATGCTGGAGTTCTTCGCCCACCCCAACTTGTTTATTGCAGCTTAT
AATGGTTACAAATAAAGCAATAGCATCACAAATTTCACAAATAAAGCATTTTTTTCACTGCA
TTCTAGTTGTGGTTTGTCCAAACTCATCAATGTATCTTATCATGTCTGTATACCGTCGACCT
CTAGCTAGAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGT
GAGCTAACTCACATTAATTGCGTTGCGCTCACTGCCCGCTTTCCAGTCGGGAAACCTGTCGT
GCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGC
TCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCGAAACCC
GACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCG
AGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCT
CTTGATCCGGCAAACAAACCACCGCTGGTAGCGGTTTTTTTGTTTGCAAGCAGCAGATTACG
CGCAGAAAAAAAGGATCTCAAGAAGATCCTTTGATCTTTTCTACGGGGTCTGACGCTCAGTG
GAACGAAAACTCACGTTAAGGGATTTTGGTCATGAGATTATCAAAAAGGATCTTCACCTAGA
TCCTTTTAAATTAAAAATGAAGTTTTAAATCAATCTAAAGTATATATGAGTAAACTTGGTCT
GACAGTTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAATAAAC
CAGCCAGCCGGAAGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCAGCTCC
GGTTCCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTT
CTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCC
ACCTGACGTC
HA-nEPEA-OGT(13) (pcDNA3.1-HA-nEPEA- [symbol] -OGT(13))
Protein sequence
(SEQ ID NO: 38)
MAYPYDVPDYAAIAMGQLVESGGGSVQAGGSLRLSCAASGIDSSSYCMGWFRQRPGKEREGVAR
INGLGGVKTAYADSVKDRFTISRDNAENTVYLQMNSLKPEDTAIYYCAAKFSPGYCGGSWSNFG
YWGQGTQVTVSSGAP [symbol] [symbol] GSMASSVGNVADSTEPTKRMLS
FQGLAELAHREYQAGDFEAAERHCMQLWRQEPDNTGVLLLLSSIHFQCRRLDRSAHFSTLAIK
QNPLLAEAYSNLGNVYKERGQLQEAIEHYRHALRLKPDFIDGYINLAAALVAAGDMEGAVQA
YVSALQYNPDLYCVRSDLGNLLKALGRLEEAKACYLKAIETQPNFAVAWSNLGCVFNAQGEI
WLAIHHFEKAVTLDPNFLDAYINLGNVLKEARIFDRAVAAYLRALSLSPNHAVVHGNLACVY
YEQGLIDLAIDTYRRAIELQPHFPDAYCNLANALKEKGSVAEAEDCYNTALRLCPTHADSLN
NLANIKREQGNIEEAVRLYRKALEVFPEFAAAHSNLASVLQQQGKLQEALMHYKEAIRISPT
FADAYSNMGNTLKEMQDVQGALQCYTRAIQINPAFADAHSNLASIHKDSGNIPEAIASYRTA
LKLKPDFPDAYCNLAHCLQIVCDWTDYDERMKKLVSIVAEQLEKNRLPSVHPHHSMLYPLSH
GFRKAIAERHGNLCLDKINVLHKPPYEHPKDLKLSDGRLRVGYVSSDFGNHPTSHLMQSIPG
MHNPDKFEVFCYALSPDDGTNFRVKVMAEANHFIDLSQIPCNGKAADRIHQDGIHILVNMNG
YTKGARNELFALRPAPIQAMWLGYPGTSGALFMDYIITDQETSPAEVAEQYSEKLAYMPHTF
FlGDHANMFPHLKKKAVIDFKSNGHIYDNRIVLNGIDLKAFLDSLPDVKIVKMKCPDGGDNP
DSSNTALNMPVIPMNTIAEAVIEMINRGQIQITINGFSISNGLATTQINNKAATGEEVPRTI
IVTTRSQYGLPEDAIVYCNFNQLYKIDPSTLQMWANILKRVPNSVLWLLRFPAVGEPNIQQY
AQNMGLPQNRIIFSPVAPKEEHVRRGQLADVCLDTPLCNGHTTGMDVLWAGTPMVTMPGETL
ASRVAASQLTCLGCLELIAKSRQEYEDIAVKLGTDLEYLKKIRGKVWKQRISSPLFNTKQYT
MELERLYLQMWEHYAAGNKPDHMIKPVEVTESA
HA-nGFP-OGT(4) (pcDNA3.1-HA-nGFP- [symbol] -OGT(4))
nucleotide sequence
(SEQ ID NO: 39)
GACGGATCGGGAGATCTCCCGATCCCCTATGGTGCACTCTCAGTACAATCTGCTCTGATGCC
GCATAGTTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTGCTTAGGG
TTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTAT
ATAAGCAGAGCTCTCTGGCTAACTAGAGAACCCACTGCTTACTGGCTTATCGAAATTAATAC
GACTCACTATAGGGAGACCCAAGCTGGCGAGCGTTTAAGCTTGAGCAATGGCATACCCATAC
GATGTTCCAGATTACGCTGCGATCGCACAGGTGCAGCTGGTGGAGTCTGGAGGAGCTCTGGT
GCAGCCTGGAGGAAGCCTGCGCCTGAGCTGTGCAGCTAGCGGATTTCCTGTGAACCGCTACA
GCATGCGCTGGTACCGCCAGGCTCCTGGTAAAGAGCGCGAGTGGGTGGCTGGAATGAGCAGC
GCTGGAGATCGCAGCAGCTACGAGGACAGCGTGAAAGGACGCTTTACAATCAGCCGCGATGA
TGCTCGCAACACAGTGTACCTGCAGATGAACTCTCTGAAACCTGAGGACACTGCTGTGTACT
ACTGTAACGTGAACGTGGGTTTCGAGTACTGGGGACAGGGAACACAGGTGACAGTGAGCTCT
GGCGCGCCA

[symbol]

[symbol]

[symbol]

[symbol]

[symbol]

[symbol]

[symbol]

GGATCCATGGCAGACTCTTTGAATAACCTTGCCAACATCAAACGGGAACAGGGCA
ACATTGAAGAGGCAGTTCGCCTGTATCGCAAAGCATTAGAAGTCTTCCCAGAGTTTGCTGCT
GCACATTCCAATTTAGCAAGTGTACTGCAACAGCAGGGCAAGCTGCAGGAAGCACTGATGCA
CTATAAAGAAGCCATACGAATTAGTCCTACATTTGCTGATGCTTATTCCAATATGGGAAACA
CTCTAAAGGAGATGCAGGATGTGCAGGGCGCTTTGCAGTGTTATACTCGTGCCATCCAGATT
AATCCTGCCTTTGCTGATGCACACAGCAATCTGGCCTCCATTCACAAGGATTCAGGGAATAT
CCCAGAAGCAATAGCTTCTTACCGCACAGCTCTGAAACTTAAGCCTGACTTTCCTGATGCTT
ATTGTAACTTGGCTCATTGCCTACAGATTGTCTGTGATTGGACAGACTATGATGAGCGGATG
AAGAAATTGGTTAGTATTGTAGCTGAGCAGCTAGAGAAGAATAGACTGCCTTCTGTCCATCC
TCACCATAGCATGCTGTACCCTCTTTCCCATGGCTTCAGGAAGGCTATTGCAGAGAGGCATG
GGAATCTCTGCTTGGATAAGATTAATGTCCTTCATAAACCACCATATGAACATCCAAAAGAC
TTGAAGCTCAGTGATGGCCGATTGCGTGTAGGCTATGTGAGTTCTGACTTCGGGAATCACCC
TACTTCACACCTTATGCAGTCTATTCCAGGCATGCATAATCCTGATAAGTTTGAGGTATTCT
GCTATGCCTTGAGCCCGGATGATGGTACAAACTTTCGAGTGAAGGTGATGGCGGAAGCCAAT
CATTTCATTGATCTTTCTCAGATTCCTTGTAATGGAAAAGCAGCCGACCGCATCCACCAAGA
TGGAATTCACATCCTTGTGAATATGAATGGGTATACCAAGGGTGCTCGGAATGAGCTCTTTG
CTCTTAGGCCAGCTCCTATTCAGGCCATGTGGCTGGGCTACCCTGGGACTAGTGGTGCACTG
TTCATGGATTACATCATCACTGATCAGGAAACTTCCCCAGCTGAAGTTGCAGAGCAGTATTC
TGAGAAACTGGCTTATATGCCCCATACTTTCTTTATTGGTGATCATGCTAATATGTTCCCTC
ACCTGAAGAAAAAAGCAGTCATCGATTTTAAATCCAATGGGCACATTTATGATAATCGGATA
GTTCTGAATGGCATCGATCTCAAAGCATTTCTCGATAGCCTACCCGATGTGAAGATTGTCAA
GATGAAATGTCCTGATGGAGGTGACAATCCAGACAGCAGTAACACAGCTCTTAATATGCCCG
TTATTCCCATGAATACGATTGCAGAAGCAGTAATTGAAATGATTAACAGAGGGCAGATTCAG
ATAACAATTAACGGATTCAGTATTAGCAATGGACTGGCGACTACACAGATTAATAATAAGGC
TGCAACCGGAGAGGAAGTTCCCCGTACCATTATTGTAACCACCCGCTCCCAGTATGGGCTAC
CAGAAGATGCCATTGTGTACTGTAACTTTAATCAGTTATATAAAATTGACCCATCTACCCTG
CAGATGTGGGCAAATATTCTGAAACGTGTGCCTAACAGCGTGCTTTGGCTGTTGCGTTTTCC
AGCAGTAGGAGAACCCAATATTCAACAATATGCACAAAATATGGGCCTTCCCCAGAACCGTA
TCATTTTCTCACCTGTGGCTCCTAAAGAGGAGCATGTCAGGAGAGGTCAGCTGGCTGATGTC
TGCCTGGATACTCCTTTGTGTAATGGACACACCACAGGGATGGATGTTCTCTGGGCAGGAAC
ACCCATGGTGACTATGCCAGGAGAGACTCTTGCCTCTCGAGTTGCAGCTTCTCAGCTTACTT
GTCTAGGATGTCTCGAGCTCATTGCTAAAAGCAGACAGGAATATGAAGACATAGCTGTGAAA
CTGGGAACCGATCTAGAATACCTGAAGAAAATTCGTGGCAAAGTCTGGAAACAGAGAATATC
TAGCCCTCTGTTCAACACCAAACAATACACAATGGAATTAGAGCGACTTTATCTGCAGATGT
GGGAGCATTATGCAGCTGGCAACAAACCTGACCACATGATTAAGCCTGTTGAAGTCACCGAG
TCAGCCGCGGCCGCTCGAGTCTAGAGGGCCCGTTTAAACCCGCTGATCAGCCGACTGTGCCT
TCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGC
AGGCATGCTGGGGATGCGGTGGGCTCTATGGCTTCTGAGGCGGAAAGAACCAGCTGGGGCTC
TAGGGGGTATCCCCACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCCCTTCC
TTTCTCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTAGGGTGATGGTTCACGTA
GTGGGCCATCGCCCTGATAGACGGTTTTTCGCCCTTTGACGTTGGAGTCCACGTTCTTTAAT
AGTGGACTCTTGTTCCAAACTGGAACAACACTCAACCCTATCTCGGTCTATTCTTTTGATTT
ATAAGGGATTTTGCCGATTTCGGCCTATTGGTTAAAAAATGAGCTGATTTAACAAAAATTTA
ACGCGAATTAATTCTGTGGAATGTGTGTCAGTTAGGGTGTGGAAAGTCCCCAGGCTCCCCAG
CAGGCAGAAGTATGCAAAGCATGCATCTCAATTAGTCAGCAACCAGGTGTGGAAAGTCCCCA
GGCTCCCCAGCAGGCAGAAGTATGCAAAGCATGCATCTCAATTAGTCAGCAACCATAGTCCC
GCCCCTAACTCCGCCCATCCCGCCCCTAACTCCGCCCAGTTCCGCCCATTCTCCGCCCCATG
GCTGACTAATTTTTTTTATTTATGCAGAGGCCGAGGCCGCCTCTGCCTCTGAGCTATTCCAG
AAGTAGTGAGGAGGCTTTTTTGGAGGCCTAGGCTTTTGCAAAAAGCTCCCGGGAGCTTGTAT
ATCCATTTTCGGATCTGATCAAGAGACAGGATGAGGATCGTTTCGCATGATTGAACAAGATG
GATTGCACGCAGGTTCTCCGGCCGCTTGGGTGGAGAGGCTATTCGGCTATGACTGGGCACAA
CAGACAATCGGCTGCTCTGATGCCGCCGTGTTCCGGCTGTCAGCGCAGGGGCGCCCGGTTCT
TTTTGTCAAGACCGACCTGTCCGGTGCCCTGAATGAACTGCAGGACGAGGCAGCGCGGCTAT
CGTGGCTGGCCACGACGGGCGTTCCTTGCGCAGCTGTGCTCGACGTTGTCACTGAAGCGGGA
AGGGACTGGCTGCTATTGGGCGAAGTGCCGGGGCAGGATCTCCTGTCATCTCACCTTGCTCC
TGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCC
GGTCTTGTCGATCAGGATGATCTGGACGAAGAGCATCAGGGGCTCGCGCCAGCCGAACTGTT
CGCCAGGCTCAAGGCGCGCATGCCCGACGGCGAGGATCTCGTCGTGACCCATGGCGATGCCT
GCTTGCCGAATATCATGGTGGAAAATGGCCGCTTTTCTGGATTCATCGACTGTGGCCGGCTG
GGTGTGGCGGACCGCTATCAGGACATAGCGTTGGCTACCCGTGATATTGCTGAAGAGCTTGG
CGGCGAATGGGCTGACCGCTTCCTCGTGCTTTACGGTATCGCCGCTCCCGATTCGCAGCGCA
TCGCCTTCTATCGCCTTCTTGACGAGTTCTTCTGAGCGGGACTCTGGGGTTCGAAATGACCG
ACCAAGCGACGCCCAACCTGCCATCACGAGATTTCGATTCCACCGCCGCCTTCTATGAAAGG
TTGGGCTTCGGAATCGTTTTCCGGGACGCCGGCTGGATGATCCTCCAGCGCGGGGATCTCAT
GCTGGAGTTCTTCGCCCACCCCAACTTGTTTATTGCAGCTTATAATGGTTACAAATAAAGCA
ATAGCATCACAAATTTCACAAATAAAGCATTTTTTTCACTGCATTCTAGTTGTGGTTTGTCC
AAACTCATCAATGTATCTTATCATGTCTGTATACCGTCGACCTCTAGCTAGAGCTTGGCGTA
ATCATGGTCATAGCTGTTTCCTGTGTGAAATTGTTATCCGCTCACAATTCCACACAACATAC
GAGCCGGAAGCATAAAGTGTAAAGCCTGGGGTGCCTAATGAGTGAGCTAACTCACATTAATT
GCGTTGCGCTCACTGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGCTTCCTCGCTCACTG
ACTCGCTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTT
ATCGCCACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTATCTGC
GCTCTGCTGAAGCCAGTTACCTTCGGAAAAAGAGTTGGTAGCTCTTGATCCGGCAAACAAAC
CACCGCTGGTAGCGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCACGTTAA
GGGATTTTGGTCATGAGATTATCAAAAAGGATCTTCACCTAGATCCTTTTAAATTAAAAATG
AAGTTTTAAATCAATCTAAAGTATATATGAGTAAACTTGGTCTGACAGTTACCAATGCTTAA
TCAGTGAGGCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTGCAATGATACC
GCGAGACCCACGCTCACCGGCTCCAGATTTATCAGCAATAAACCAGCCAGCCGGAAGGGCCG
AGCGCAGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCAAGGC
GAGTTACATGATCCCCCATGTTGTGCAAAAAAGCGGTTAGCTCCTTCGGTCCTCCGATCGTT
GTCAGAAGTAAGTTGGCCGCAGTGTTATCACTCATGGTTATGGCAGCACTGCATAATTCTCT
TACTGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACCGCG
CCACATAGCAGAACTTTAAAAGTGCTCATCATTGGAAAACGTTCTTCGGGGCGAAAACTCTC
AAGGATCTTACCGCTGTTGAGATCCAGTTCGATGTAACCCACTCGTGCACCCAACTGATCTT
CAGCATCTTTTACTTTCACCAGCGTTTCTGGGTGAGCAAAAACAGGAAGGCAAAATGCCGCA
AAAAAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGAAAA
ATAAACAAATAGGGGTTCCGCGCACATTTCCCCGAAAAGTGCCACCTGACGTC
HA-nGFP-OGT(4) (pcDNA3.1-HA-nGFP- [symbol] -OGT(4))
protein sequence
(SEQ ID NO: 40)
MAYPYDVPDYAAIAQVQLVESGGALVQPGGSLRLSCAASGFPVNRYSMRWYRQAPGKEREWVAG
MSSAGDRSSYEDSVKGRFTISRDDARNTVYLQMNSLKPEDTAVYYCNVNVGFEYWGQGTQVTVS
SGAP [symbol] [symbol] GSMADSLNNLANIKREQGNIEEAVRLYRKALE
VFPEFAAAHSNLASVLQQQGKLQEALMHYKEAIRISPTFADAYSNMGNTLKEMQDVQGALQCY
TRAIQINPAFADAHSNLASIHKDSGNIPEAIASYRTALKLKPDFPDAYCNLAHCLQIVCDWTD
YDERMKKLVSIVAEQLEKNRLPSVHPHHSMLYPLSHGFRKAIAERHGNLCLDKINVLHKPPY
EHPKDLKLSDGRLRVGYVSSDFGNHPTSHLMQSIPGMHNPDKFEVFCYALSPDDGTNFRVKV
MAEANHFIDLSQIPCNGKAADRIHQDGIHILVNMNGYTKGARNELFALRPAPIQAMWLGYPG
TSGALFMDYIITDQETSPAEVAEQYSEKLAYMPHTFFIGDHANMFPHLKKKAVIDFKSNGHI
YDNRIVLNGIDLKAFLDSLPDVKIVKMKCPDGGDNPDSSNTALNMPVIPMNTIAEAVIEMIN
RGQIQITINGFSISNGLATTQINNKAATGEEVPRTIIVTTRSQYGLPEDAIVYCNFNQLYKI
DPSTLQMWANILKRVPNSVLWLLRFPAVGEPNIQQYAQNMGLPQNRIIFSPVAPKEEHVRRG
QLADVCLDTPLCNGHTTGMDVLWAGTPMVTMPGETLASRVAASQLTCLGCLELIAKSRQEYE
DIAVKLGTDLEYLKKIRGKVWKQRISSPLFNTKQYTMELERLYLQMWEHYAAGNKPDHMIKP
VEVTESAAAARV
HA-nGFP-OGT(13) (pcDNA3.1-HA-nGFP- [symbol] -OGT(13))
nucleotide sequence
(SEQ ID NO: 41)
GACGGATCGGGAGATCTCCCGATCCCCTATGGTGCACTCTCAGTACAATCTGCTCTGATGCC
GCATAGTTAAGCCAGTATCTGCTCCCTGCTTGTGTGTTGGAGGTCGCTGAGTAGTGCGCGAG
CAAAATTTAAGCTACAACAAGGCAAGGCTTGACCGACAATTGCATGAAGAATCTGCTTAGGG
TTAGGCGTTTTGCGCTGCTTCGCGATGTACGGGCCAGATATACGCGTTGACATTGATTATTG
ACTAGTTATTAATAGTAATCAATTACGGGGTCATTAGTTCATAGCCCATATATGGAGTTCCG
CGTTACATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTAC
GCCCCCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCT
CCACCCCATTGACGTCAATGGGAGTTTGTTTTGGCACCAAAATCAACGGGACTTTCCAAAAT
GTCGTAACAACTCCGCCCCATTGACGCAAATGGGCGGTAGGCGTGTACGGTGGGAGGTCTAT
ATAAGCAGAGCTCTCTGGCTAACTAGAGAACCCACTGCTTACTGGCTTATCGAAATTAATAC
GACTCACTATAGGGAGACCCAAGCTGGCGAGCGTTTAAGCTTGAGCAATGGCATACCCATAC
GATGTTCCAGATTACGCTGCGATCGCACAGGTGCAGCTGGTGGAGTCTGGAGGAGCTCTGGT
GCAGCCTGGAGGAAGCCTGCGCCTGAGCTGTGCAGCTAGCGGATTTCCTGTGAACCGCTACA
GCATGCGCTGGTACCGCCAGGCTCCTGGTAAAGAGCGCGAGTGGGTGGCTGGAATGAGCAGC
GCTGGAGATCGCAGCAGCTACGAGGACAGCGTGAAAGGACGCTTTACAATCAGCCGCGATGA
TGCTCGCAACACAGTGTACCTGCAGATGAACTCTCTGAAACCTGAGGACACTGCTGTGTACT
ACTGTAACGTGAACGTGGGTTTCGAGTACTGGGGACAGGGAACACAGGTGACAGTGAGCTCT
GGCGCGCCA

[symbol]

[symbol]

[symbol]

[symbol]

[symbol]

[symbol]

[symbol]

GGATCCATGGCGTCTTCCGTGGGCAACGTGGCCGACAGTACAGAACCAACGAAAC
GTATGCTTTCCTTCCAAGGGTTAGCTGAGTTGGCACATCGAGAATATCAGGCAGGAGATTTT
GAGGCAGCTGAGAGACACTGCATGCAGCTCTGGAGACAAGAGCCTGACAATACTGGTGTTCT
TTTATTACTTTCATCTATACACTTCCAGTGTCGAAGGCTGGACAGATCTGCTCATTTTAGCA
CCTTGGCAATTAAACAGAATCCCCTTCTAGCAGAAGCCTATTCGAATTTAGGAAATGTGTAC
AAGGAAAGAGGGCAGTTGCAGGAAGCAATCGAGCATTATCGACATGCCTTGCGGCTGAAGCC
TGATTTCATTGATGGTTATATTAACCTGGCAGCAGCCTTGGTAGCAGCAGGTGACATGGAAG
GAGCAGTACAAGCCTATGTCTCTGCTCTTCAGTACAATCCTGATTTGTACTGTGTTCGCAGT
GACCTGGGGAACCTGCTCAAAGCCCTGGGTCGCTTGGAAGAAGCCAAGGCATGTTATTTGAA
AGCAATTGAGACGCAACCAAACTTTGCAGTAGCCTGGAGTAATCTCGGCTGTGTTTTCAATG
CACAAGGGGAGATTTGGCTGGCTATTCATCACTTTGAAAAGGCTGTCACCCTTGACCCAAAT
TTTCTGGATGCTTATATCAATTTAGGAAATGTCTTGAAAGAGGCACGCATTTTTGACAGAGC
TGTCGCAGCTTATCTTCGTGCCTTAAGTTTGAGCCCAAATCATGCGGTGGTGCACGGCAACC
TGGCTTGTGTGTACTACGAGCAAGGCCTAATAGACCTGGCCATTGATACCTACAGGAGAGCT
ATCGAACTGCAACCCCATTTCCCCGATGCTTACTGCAACCTAGCAAATGCTCTCAAAGAGAA
GGGCAGTGTTGCTGAAGCAGAAGACTGTTATAACACAGCTCTTCGTCTGTGTCCTACTCATG
CAGACTCTTTGAATAACCTTGCCAACATCAAACGGGAACAGGGCAACATTGAAGAGGCAGTT
CGCCTGTATCGCAAAGCATTAGAAGTCTTCCCAGAGTTTGCTGCTGCACATTCCAATTTAGC
AAGTGTACTGCAACAGCAGGGCAAGCTGCAGGAAGCACTGATGCACTATAAAGAAGCCATAC
GAATTAGTCCTACATTTGCTGATGCTTATTCCAATATGGGAAACACTCTAAAGGAGATGCAG
GATGTGCAGGGCGCTTTGCAGTGTTATACTCGTGCCATCCAGATTAATCCTGCCTTTGCTGA
TGCACACAGCAATCTGGCCTCCATTCACAAGGATTCAGGGAATATCCCAGAAGCAATAGCTT
CTTACCGCACAGCTCTGAAACTTAAGCCTGACTTTCCTGATGCTTATTGTAACTTGGCTCAT
TGCCTACAGATTGTCTGTGATTGGACAGACTATGATGAGCGGATGAAGAAATTGGTTAGTAT
TGTAGCTGAGCAGCTAGAGAAGAATAGACTGCCTTCTGTCCATCCTCACCATAGCATGCTGT
ACCCTCTTTCCCATGGCTTCAGGAAGGCTATTGCAGAGAGGCATGGGAATCTCTGCTTGGAT
AAGATTAATGTCCTTCATAAACCACCATATGAACATCCAAAAGACTTGAAGCTCAGTGATGG
CCGATTGCGTGTAGGCTATGTGAGTTCTGACTTCGGGAATCACCCTACTTCACACCTTATGC
AGTCTATTCCAGGCATGCATAATCCTGATAAGTTTGAGGTATTCTGCTATGCCTTGAGCCCG
GATGATGGTACAAACTTTCGAGTGAAGGTGATGGCGGAAGCCAATCATTTCATTGATCTTTC
TCAGATTCCTTGTAATGGAAAAGCAGCCGACCGCATCCACCAAGATGGAATTCACATCCTTG
TGAATATGAATGGGTATACCAAGGGTGCTCGGAATGAGCTCTTTGCTCTTAGGCCAGCTCCT
ATTCAGGCCATGTGGCTGGGCTACCCTGGGACTAGTGGTGCACTGTTCATGGATTACATCAT
CACTGATCAGGAAACTTCCCCAGCTGAAGTTGCAGAGCAGTATTCTGAGAAACTGGCTTATA
TGCCCCATACTTTCTTTATTGGTGATCATGCTAATATGTTCCCTCACCTGAAGAAAAAAGCA
GTCATCGATTTTAAATCCAATGGGCACATTTATGATAATCGGATAGTTCTGAATGGCATCGA
TCTCAAAGCATTTCTCGATAGCCTACCCGATGTGAAGATTGTCAAGATGAAATGTCCTGATG
GAGGTGACAATCCAGACAGCAGTAACACAGCTCTTAATATGCCCGTTATTCCCATGAATACG
ATTGCAGAAGCAGTAATTGAAATGATTAACAGAGGGCAGATTCAGATAACAATTAACGGATT
CAGTATTAGCAATGGACTGGCGACTACACAGATTAATAATAAGGCTGCAACCGGAGAGGAAG
TTCCCCGTACCATTATTGTAACCACCCGTTCCCAGTATGGGCTACCAGAAGATGCCATTGTG
TACTGTAACTTTAATCAGTTATATAAAATTGACCCATCTACCCTGCAGATGTGGGCAAATAT
TCTGAAACGTGTGCCTAACAGCGTGCTTTGGCTGTTGCGTTTTCCAGCAGTAGGAGAACCCA
ATATTCAACAATATGCACAAAATATGGGCCTTCCCCAGAACCGTATCATTTTCTCACCTGTG
GCTCCTAAAGAGGAGCATGTCAGGAGAGGTCAGCTGGCTGATGTCTGCCTGGATACTCCTTT
GTGTAATGGACACACCACAGGGATGGATGTTCTCTGGGCAGGAACACCCATGGTGACTATGC
CAGGAGAGACTCTTGCCTCTCGAGTTGCAGCTTCTCAGCTTACTTGTCTAGGATGTCTCGAG
CTCATTGCTAAAAGCAGACAGGAATATGAAGACATAGCTGTGAAACTGGGAACCGATCTAGA
ATACCTGAAGAAAATTCGTGGCAAAGTCTGGAAACAGAGAATATCTAGCCCTCTGTTCAACA
CCAAACAATACACAATGGAATTAGAGCGACTTTATCTGCAGATGTGGGAGCATTATGCAGCT
GGCAACAAACCTGACCACATGATTAAGCCTGTTGAAGTCACCGAGTCAGCCTAAGCGGCCGC
TCGAGTCTAGAGGGCCCGTTTAAACCCGCTGATCAGCCGACTGTGCCTTCTAGTTGCCAGCC
ATCTGTTGTTTGCCCCTCCCCCGTGCCTTCCTTGACCCTGGAAGGTGCCACTCCCACTGTCC
TTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGA
TGCGGTGGGCTCTATGGCTTCTGAGGCGGAAAGAACCAGCTGGGGCTCTAGGGGGTATCCCC
ACGCGCCCTGTAGCGGCGCATTAAGCGCGGCGGGTGTGGTGGTTACGCGCAGCGTGACCGCT
ACACTTGCCAGCGCCCTAGCGCCCGCTCCTTTCGCTTTCTTCCCTTCCTTTCTCGCCACGTT
CGCCGGCTTTCCCCGTCAAGCTCTAAATCGGGGGCTCCCTTTAGGGTTCCGATTTAGTGCTT
TACGGCACCTCGACCCCAAAAAACTTGATTAGGGTGATGGTTCACGTAGTGGGCCATCGCCC
TGATAGACGGTTTTTCGCCCTTTGACGTTGGAGTCCACGTTCTTTAATAGTGGACTCTTGTT
CCAAACTGGAACAACACTCAACCCTATCTCGGTCTATTCTTTTGATTTATAAGGGATTTTGC
CGATTTCGGCCTATTGGTTAAAAAATGAGCTGATTTAACAAAAATTTAACGCGAATTAATTC
TGTGGAATGTGTGTCAGTTAGGGTGTGGAAAGTCCCCAGGCTCCCCAGCAGGCAGAAGTATG
CAAAGCATGCATCTCAATTAGTCAGCAACCAGGTGTGGAAAGTCCCCAGGCTCCCCAGCAGG
CAGAAGTATGCAAAGCATGCATCTCAATTAGTCAGCAACCATAGTCCCGCCCCTAACTCCGC
CCATCCCGCCCCTAACTCCGCCCAGTTCCGCCCATTCTCCGCCCCATGGCTGACTAATTTTT
TTTATTTATGCAGAGGCCGAGGCCGCCTCTGCCTCTGAGCTATTCCAGAAGTAGTGAGGAGG
CTTTTTTGGAGGCCTAGGCTTTTGCAAAAAGCTCCCGGGAGCTTGTATATCCATTTTCGGAT
CTGATCAAGAGACAGGATGAGGATCGTTTCGCATGATTGAACAAGATGGATTGCACGCAGGT
TCTCCGGCCGCTTGGGTGGAGAGGCTATTCGGCTATGACTGGGCACAACAGACAATCGGCTG
CTCTGATGCCGCCGTGTTCCGGCTGTCAGCGCAGGGGCGCCCGGTTCTTTTTGTCAAGACCG
ACCTGTCCGGTGCCCTGAATGAACTGCAGGACGAGGCAGCGCGGCTATCGTGGCTGGCCACG
ACGGGCGTTCCTTGCGCAGCTGTGCTCGACGTTGTCACTGAAGCGGGAAGGGACTGGCTGCT
ATTGGGCGAAGTGCCGGGGCAGGATCTCCTGTCATCTCACCTTGCTCCTGCCGAGAAAGTAT
CCATCATGGCTGATGCAATGCGGCGGCTGCATACGCTTGATCCGGCTACCTGCCCATTCGAC
CACCAAGCGAAACATCGCATCGAGCGAGCACGTACTCGGATGGAAGCCGGTCTTGTCGATCA
GGATGATCTGGACGAAGAGCATCAGGGGCTCGCGCCAGCCGAACTGTTCGCCAGGCTCAAGG
CGCGCATGCCCGACGGCGAGGATCTCGTCGTGACCCATGGCGATGCCTGCTTGCCGAATATC
ATGGTGGAAAATGGCCGCTTTTCTGGATTCATCGACTGTGGCCGGCTGGGTGTGGCGGACCG
CTATCAGGACATAGCGTTGGCTACCCGTGATATTGCTGAAGAGCTTGGCGGCGAATGGGCTG
ACCGCTTCCTCGTGCTTTACGGTATCGCCGCTCCCGATTCGCAGCGCATCGCCTTCTATCGC
CTTCTTGACGAGTTCTTCTGAGCGGGACTCTGGGGTTCGAAATGACCGACCAAGCGACGCCC
AACCTGCCATCACGAGATTTCGATTCCACCGCCGCCTTCTATGAAAGGTTGGGCTTCGGAAT
CGTTTTCCGGGACGCCGGCTGGATGATCCTCCAGCGCGGGGATCTCATGCTGGAGTTCTTCG
CCCACCCCAACTTGTTTATTGCAGCTTATAATGGTTACAAATAAAGCAATAGCATCACAAAT
TTCACAAATAAAGCATTTTTTTCACTGCATTCTAGTTGTGGTTTGTCCAAACTCATCAATGT
ATCTTATCATGTCTGTATACCGTCGACCTCTAGCTAGAGCTTGGCGTAATCATGGTCATAGC
TGTTTCCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCCAACGCGCGG
GGAGAGGCGGTTTGCGTATTGGGCGCTCTTCCGCTTCCTCGCTCACTGACTCGCTGCGCTCG
GTCGTTCGGCTGCGGCGAGCGGTATCAGCTCACTCAAAGGCGGTAATACGGTTATCCACAGA
ATCAGGGGATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTTTCCCCC
TGGAAGCTCCCTCGTGCGCTCTCCTGTTCCGACCCTGCCGCTTACCGGATACCTGTCCGCCT
TTCTCCCTTCGGGAAGCGTGGCGCTTTCTCATAGCTCACGCTGTAGGTATCTCAGTTCGGTG
TAGGTCGTTCGCTCCAAGCTGGGCTGTGTGCACGAACCCCCCGTTCAGCCCGACCGCTGCGC
CTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAG
TGGTGGCCTAACTACGGCTACACTAGAAGAACAGTATTTGGTATCTGCGCTCTGCTGAAGCC
AGTTACCTTCGGAAAAAGAGTTGGTAGCTCTTGATCCGGCAAACAAACCACCGCTGGTAGCG
GTTTTTTTGTTTGCAAGCAGCAGATTACGCGCAGAAAAAAAGGATCTCAAGAAGATCCTTTG
ATCTTTTCTACGGGGTCTGACGCTCAGTGGAACGAAAACTCACGTTAAGGGATTTTGGTCAT
GAGATTATCAAAAAGGATCTTCACCTAGATCCTTTTAAATTAAAAATGAAGTTTTAAATCAA
TCTAAAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGATAAC
TACGATACGGGAGGGCTTACCATCTGGCCCCAGTGCTGCAATGATACCGCGAGACCCACGCT
CACCGGCTCCAGATTTATCAGCAATAAACCAGCCAGCCGGAAGGGCCGAGCGCAGAAGTGGT
CCTGCAACTTTATCCGCCTCCATCCAGTCTATTAATTGTTGCCGGGAAGCTAGAGTAAGTAG
TTCGCCAGTTAATAGTTTGCGCAACGTTGTTGCCATTGCTACAGGCATCGTGGTGTCACGCT
CGTCGTTTGGTATGGCTTCATTCAGCTCCGGTTCCCAACGATCAAGGCGAGTTACATGATCC
CCCATGTTGTGCAAAAAAGCGGTTAGCTCCTTCGGTCCTCCGATCGTTGTCAGAAGTAAGTT
GGCCGCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTGTATG
CGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGC
TGTTGAGATCCAGTTCGATGTAACCCACTCGTGCACCCAACTGATCTTCAGCATCTTTTACT
TTCACCAGCGTTTCTGGGTGAGCAAAAACAGGAAGGCAAAATGCCGCAAAAAAGGGAATAAG
GGCGACACGGAAATGTTGAATACTCATACTCTTCCTTTTTCAATATTATTGAAGCATTTATC
AGGGTTATTGTCTCATGAGCGGATACATATTTGAATGTATTTAGAAAAATAAACAAATAGGG
GTTCCGCGCACATTTCCCCGAAAAGTGCCACCTGACGTC
HA-nGFP-OGT(13) (pcDNA3.1-HA-nGFP- [symbol] -OGT(13))
protein sequence
(SEQ ID NO: 42)
MAYPYDVPDYAAIAQVQLVESGGALVQPGGSLRLSCAASGFPVNRYSMRWYRQAPGKEREWVAG
MSSAGDRSSYEDSVKGRFTISRDDARNTVYLQMNSLKPEDTAVYYCNVNVGFEYWGQGTQVTVS
SGAP [symbol] [symbol] GSMASSVGNVADSTEPTKRMLSFQGLAELAHRE
YQAGDFEAAERHCMQLWRQEPDNTGVLLLLSSIHFQCRRLDRSAHFSTLAIKQNPLLAEAYSN
LGNVYKERGQLQEAIEHYRHALRLKPDFIDGYINLAAALVAAGDMEGAVQAYVSALQYNPDL
YCVRSDLGNLLKALGRLEEAKACYLKAIETQPNFAVAWSNLGCVFNAQGEIWLAIHHFEKAV
TLDPNFLDAYINLGNVLKEARIFDRAVAAYLRALSLSPNHAVVHGNLACVYYEQGLIDLAID
TYRRAIELQPHFPDAYCNLANALKEKGSVAEAEDCYNTALRLCPTHADSLNNLANIKREQGN
IEEAVRLYRKALEVFPEFAAAHSNLASVLQQQGKLQEALMHYKEAIRISPTFADAYSNMGNT
LKEMQDVQGALQCYTRAIQINPAFADAHSNLASIHKDSGNIPEAIASYRTALKLKPDFPDAY
CNLAHCLQIVCDWTDYDERMKKLVSIVAEQLEKNRLPSVHPHHSMLYPLSHGFRKAIAERHG
NLCLDKINVLHKPPYEHPKDLKLSDGRLRVGYVSSDFGNHPTSHLMQSIPGMHNPDKFEVFC
YALSPDDGTNFRVKVMAEANHFIDLSQIPCNGKAADRIHQDGIHILVNMNGYTKGARNELFA
LRPAPIQAMWLGYPGTSGALFMDYIITDQETSPAEVAEQYSEKLAYMPHTFFIGDHANMFPH
LKKKAVIDFKSNGHIYDNRIVLNGIDLKAFLDSLPDVKIVKMKCPDGGDNPDSSNTALNMPV
IPMNTIAEAVIEMINRGQIQITINGFSISNGLATTQINNKAATGEEVPRTIIVTTRSQYGLP
EDAIVYCNFNQLYKIDPSTLQMWANILKRVPNSVLWLLRFPAVGEPNIQQYAQNMGLPQNRI
IFSPVAPKEEHVRRGQLADVCLDTPLCNGHTTGMDVLWAGTPMVTMPGETLASRVAASQLTC
LGCLELIAKSRQEYEDIAVKLGTDLEYLKKIRGKVWKQRISSPLFNTKQYTMELERLYLQMW
EHYAAGNKPDHMIKPVEVTESA

### EQUIVALENTS AND SCOPE, INCORPORATION BY REFERENCE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

All publications, patents and sequence database entries mentioned herein, including those items listed above, are hereby incorporated by reference in their entirety as if each individual publication or patent was specifically and individually indicated to be incorporated by reference. In case of conflict, the present application, including any definitions herein, will control.

What is claimed is:

1. A fusion protein comprising (i) a nanobody, and (ii) a glycan modifying enzyme.

2. The fusion protein of claim 1, wherein the nanobody is fused to the N-terminal domain of the enzyme.

3. The fusion protein of claim 1, wherein the nanobody is fused to the C-terminus of the enzyme.

4. The fusion protein of claim 1, wherein the enzyme is a glycosyl transferase.

5. The fusion protein of claim 4, wherein the enzyme is O-GlcNAc transferase (OGT).

6. The fusion protein of claim 1, wherein the enzyme is a glycosyl hydrolase.

7. The fusion protein of claim 6, wherein the enzyme is O-GlcNAcase (OGA).

8. The fusion protein of claim 5, wherein the enzyme comprises (i) a catalytic domain, and optionally, (ii) a tetratricopeptide repeat (TPR) domain.

9. The fusion protein of claim 8, wherein the number of tetratricopeptide repeat (TPR) domains is selected from a group consisting of 0, 1, 2, 3, 4, 5, 6, 7, 8, 9, 10, 11, 12, and 13.

10. The fusion protein of any one of claims 1-9, wherein the nanobody binds a cell surface protein.

11. The fusion protein of any one of claims 1-9, wherein the nanobody binds a green fluorescent protein (GFP).

12. The fusion protein of claim 1, wherein the nanobody binds a specific peptide tag.

13. The fusion protein of claim 12, wherein the specific peptide tag is a four-amino acid tag.

14. The fusion protein of claim 13, wherein the four-amino acid tag is EPEA.

15. The fusion protein of claim 14, wherein the nanobody binds the four-amino acid EPEA tag (nEPEA).

16. The fusion protein of any one of claims 1-9, wherein the nanobody binds beta-catenin.

17. The fusion protein of any one of claims 1-9, wherein the nanobody binds TET3.

18. The fusion protein of any one of claims 1-9, wherein the nanobody binds alpha-synuclein.

19. The fusion protein of any one of claims 1-9, wherein the nanobody binds Tau.

20. The fusion protein of any one of claims 1 to 19, wherein the nanobody and the glycan modifying enzyme are fused via a linker.

21. A method of glycosylating a protein, the method comprising contacting a target protein with a fusion protein of any one of claims 1 to 20.

22. The method of claim 21, wherein the target protein is selected from the group consisting of nuclear proteins, cytosolic proteins, and mitochondrial proteins.

23. The method of claim 21, wherein the target protein is selected from the group consisting of transcription factors, kinases, oxidoreductases, nucleoporins, and nuleosomes.

24. The method of claim 23, wherein the transcription factor is selected from the group consisting of c-JUN, JUNB, IKZF1, and STAT1.

25. The method of claim 23, wherein the kinase is Zap70.

26. The method of claim 23, wherein the oxidoreductase is TET3.

27. The method of claim 23, wherein the nucleoporin is selected from the group consisting of Nup35 and Nup62.

28. The method of claim 23, wherein the nucleosome is selected from the group consisting of H2B, H3, and H4.

29. A method of glycosylating a protein, the method comprising:
contacting a target protein with a fusion protein of any one of claims 1 to 20 in the presence of a glycosyl donor molecule, thereby installing the sugar moiety from the glycosyl donor molecule on the target protein.

30. A method of glycosylating a protein, the method comprising:
contacting a target protein with a fusion protein of any one of claims 1 to 20 in the presence of a O-linked N-acetyl glucosamine donor molecule, thereby installing a O-linked N-acetyl glucosamine on the target protein via the addition of a glucosamine monosaccharide attached to serine or threonine.

31. The method according to claim 29 or 30, wherein the target protein is alpha-synuclein.

32. The method according to claim 29 or 30, wherein the target protein is Tau.

33. The method according to claim 29 or 30, wherein the target protein is TET3.

34. The method according to claim 29 or 30, wherein the target protein is beta-catenin.

35. A method of removing a sugar from a protein, the method comprising:
contacting a target protein containing a sugar with a fusion protein of any one of claims 1 to 20, thereby excising a sugar moiety from the target protein.

36. A method of removing a sugar from a protein, the method comprising:
contacting a target protein containing an O-linked N-acetyl glucosamine with a fusion protein of any one of claims 1 to 20, thereby excising an O-linked N-acetyl glucosamine from a serine or threonine residue of the target protein.

37. The method according to claim 35 or 36, wherein the target protein is alpha-synuclein.

38. The method according to claim 35 or 36, wherein the target protein is Tau.

39. A method of studying the effect of glycosylation in a cell using the fusion protein of any one of claims 1 to 20.

40. A method of treating a disease, the method comprising administering a fusion protein of any one of claims 1 to 20 to a subject in need thereof.

41. A method of diagnosing a subject with a disease, the method comprising administering a fusion protein of any one of claims 1 to 20 to a subject.

42. A method of treating a subject suffering from or susceptible to a neurodegenerative disease, the method comprising administering an effective amount of the fusion protein of any one of claims 1 to 20.

43. The method of claim 42, wherein the neurodegenerative disease is selected from the group consisting of Parkinson's disease, Huntington's disease, Alzheimer's disease, dementia, and multiple system atrophy.

44. The method according to claim 43, wherein the neurodegenerative disease is Parkinson's disease.

45. The method according to claim 43, wherein the neurodegenerative disease is Huntington's disease.

46. A method of treating a subject suffering from or susceptible to a psychotic disorder, the method comprising administering an effective amount of the fusion protein of any one of claims 1 to 20.

47. The method of claim 46, wherein the psychotic disorder is schizophrenia.

48. A method of treating a subject suffering from or susceptible to epilepsy, the method comprising administering an effective amount of the fusion protein of any one of claims 1 to 20.

49. A method of treating a subject suffering from or susceptible to a sleep disorder, the method comprising administering an effective amount of the fusion protein of any one of claims 1 to 20.

50. A method of treating a subject suffering from or susceptible to an addiction, the method comprising administering an effective amount of the fusion protein of any one of claims 1 to 20.

51. A pharmaceutical composition comprising a compound of any one of claims 1 to 20, and a pharmaceutically acceptable excipient.

52. A kit comprising:
(a) a fusion protein of any one of claims 1 to 20; and
(b) either a glycosyl donor molecule or a glycosyl acceptor molecule.

53. The kit according to claim 52, wherein the glycosyl donor molecule is uridine diphosphate N-acetylglucosamine.

54. A kit comprising:
(a) a vector for expressing a fusion protein of any one of claims 1 to 20; and
(b) either a glycosyl donor molecule or a glycosyl acceptor molecule.

55. The kit according to claim 54, wherein the glycosyl donor molecule is uridine diphosphate N-acetylglucosamine.

56. A polynucleotide encoding the fusion protein of any one of claims 1 to 20.

57. A vector comprising the polynucleotide of claim 56.

58. A cell comprising the fusion protein of any one of claims 1 to 20.

59. A cell comprising the nucleic acid molecule encoding the fusion protein of any one of claims 1 to 20.


SEQUENCE LISTING (43 sequences)

SEQ ID NO: 1 - DNA, 101 bp, Artificial Sequence
  Synthetic Polynucleotide
cccaagctggcgagcgtttaagcttgagcaatggcccatacccatacgatgttccagattacgctgcgatcgcacaggtgcagctggtggagtctggagga

SEQ ID NO: 2 - DNA, 97 bp, Artificial Sequence
  Synthetic Polynucleotide
ggatccctttgcagctgcctcctttgcagctgcctcctttgcagctgcctcctttgcagctgcctctggcgcgccagagctcactgtcacctgtgtt

SEQ ID NO: 3 - DNA, 60 bp, Artificial Sequence
  Synthetic Polynucleotide
aaaggaggcagctgcaaaggaggcagctgcaaagggatccatggcgtcttccgtgggcaa

SEQ ID NO: 4 - DNA, 78 bp, Artificial Sequence
  Synthetic Polynucleotide
cgggtttaaacgggccctctagactcgagcggccgcttaggctgactcggtgacttcaacaggcttaatcatgtggtc

SEQ ID NO: 5 - DNA, 34 bp, Artificial Sequence
  Synthetic Polynucleotide
tacgctgcgatcgcaatgggccagctggtggaga

SEQ ID NO: 6 - DNA, 34 bp, Artificial Sequence
  Synthetic Polynucleotide
ctggcgcgccagagctcacagtaacctgggtgcc

SEQ ID NO: 7 - DNA, 50 bp, Artificial Sequence
  Synthetic Polynucleotide
aaagggatccatggcagactctttgaataaccttgccaacatcaaacggg

SEQ ID NO: 8 - DNA, 43 bp, Artificial Sequence
  Synthetic Polynucleotide
gcaaagggatcccctgatgcttattgtaacttggctcattgcc

SEQ ID NO: 9 - DNA, 42 bp, Artificial Sequence
  Synthetic Polynucleotide
ttacgctgcgatcgcaatggcgtcttccgtgggcaacgtggc

SEQ ID NO: 10 - DNA, 72 bp, Artificial Sequence
  Synthetic Polynucleotide
tatagcggccgctggcgcgccttaggctgactcggtgacttcaacaggcttaatcatgtggtcaggtttgtt

SEQ ID NO: 11 - DNA, 62 bp, Artificial Sequence
  Synthetic Polynucleotide
acgctgcgatcgcaatggcagactctttgaataaccttgccaacatcaaacgggaacagggc

SEQ ID NO: 12 - DNA, 85 bp, Artificial Sequence
  Synthetic Polynucleotide
cgctgcgatcgcaatgcctgatgcttattgtaacttggctcattgcctacagattgtctgtgattggacagactatgatgagcgg

SEQ ID NO: 13 - DNA, 44 bp, Artificial Sequence
  Synthetic Polynucleotide
acttaagcttgggcgatcgcaatggcaagcgggtttaattttgg

SEQ ID NO: 14 - DNA, 54 bp, Artificial Sequence
  Synthetic Polynucleotide
ctctagactcgagttatgcttcaggttccttatcgtcgtcatccttgtagtctg

SEQ ID NO: 15 - DNA, 36 bp, Artificial Sequence
  Synthetic Polynucleotide
caggcgatcgcaatggcaccagagccagcgaagtct

SEQ ID NO: 16 - DNA, 34 bp, Artificial Sequence
  Synthetic Polynucleotide
gtctggcgcgcccttagcgctggtgtacttggtg

SEQ ID NO: 17 - DNA, 41 bp, Artificial Sequence
  Synthetic Polynucleotide
agcaggcgatcgcaatggctcgtactaaacagacagctcgg

SEQ ID NO: 18 - DNA, 29 bp, Artificial Sequence
  Synthetic Polynucleotide
tctggcgcgcccgctctttctccgcgaat

SEQ ID NO: 19 - DNA, 34 bp, Artificial Sequence
  Synthetic Polynucleotide
agcaggcgatcgcaatgtctggccgcggcaaagg

SEQ ID NO: 20 - DNA, 34 bp, Artificial Sequence
  Synthetic Polynucleotide
aggcgatcgcaatggcagcctttgcagtggaacc

SEQ ID NO: 21 - DNA, 35 bp, Artificial Sequence
  Synthetic Polynucleotide
tctggcgcgccccagccaaacatgtactccattgc

SEQ ID NO: 22 - DNA, 36 bp, Artificial Sequence
  Synthetic Polynucleotide
agcaggcgatcgcaatggactcagggccagtgtacc

SEQ ID NO: 23 - DNA, 29 bp, Artificial Sequence
  Synthetic Polynucleotide
tctggcgcgccgatccagcggctgtaggg

SEQ ID NO: 24 - DNA, 33 bp, Artificial Sequence
  Synthetic Polynucleotide
gacacacctgccaagagagcccaggccgagttc

SEQ ID NO: 25 - DNA, 50 bp, Artificial Sequence
  Synthetic Polynucleotide
cattgcgatcgcccaagcttaagtttaaacgctagccagcttgggtctcc

SEQ ID NO: 26 - DNA, 40 bp, Artificial Sequence
  Synthetic Polynucleotide
ctggcaggcgatcgcaatgactgcaaagatggaaacgacc

SEQ ID NO: 27 - DNA, 36 bp, Artificial Sequence
  Synthetic Polynucleotide
tctggcgcgccaaatgtttgcaactgctgcgttagc

SEQ ID NO: 28 - DNA, 42 bp, Artificial Sequence
  Synthetic Polynucleotide
tggcaggcgatcgcaatgtgcactaaaatggaacagcccttc

SEQ ID NO: 29 - DNA, 31 bp, Artificial Sequence
  Synthetic Polynucleotide
tagtctggcgcgccgaaggcgtgtcccttga

SEQ ID NO: 30 - DNA, 47 bp, Artificial Sequence
  Synthetic Polynucleotide
ctggcaggcgatcgcaatggatgctgatgagggtcaagacatgtccc

SEQ ID NO: 31 - DNA, 34 bp, Artificial Sequence
  Synthetic Polynucleotide
tagtctggcgcgccgctcatgtggaagcggtgct

SEQ ID NO: 32 - DNA, 31 bp, Artificial Sequence
  Synthetic Polynucleotide
gcaggcgatcgcaatgtctcagtggtacgaa

SEQ ID NO: 33 - DNA, 32 bp, Artificial Sequence
  Synthetic Polynucleotide
ctggcgcgcctactgtgttcatcatactgtcg

SEQ ID NO: 34 - DNA, 411 bp, Artificial Sequence
  Synthetic Polynucleotide
atgggccagctggtggagagcggcggcggcagcgtgcaggccggcggcagcctgaggctgagctgcgccgccagcggcatcgacagcagcagctactgcatgggctggttcaggcagaggcccggcaaggagagggagggcgtggccaggatcaacggcctgggcggcgtgaagaccgcctacgccgacagcgtgaaggacaggttcaccatcagcagggacaacgccgagaacaccgtgtacctgcagatgaacagcctgaagcccgaggacaccgccatctactactgcgccgccaagttcagccccggctactgcggcggcagctggagcaacttcggctactggggccagggcacccaggttactgtgagctctcaccaccatcatcatcatctgcccgagaccggc

SEQ ID NO: 35 - DNA, 8025 bp, Artificial Sequence
  Synthetic Polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggcgagcgtttaagcttgagcaatggcatacccatacgatgttccagattacgctgcgatcgcaatgggccagctggtggagagcggcggcggcagcgtgcaggccggcggcagcctgaggctgagctgcgccgccagcggcatcgacagcagcagctactgcatgggctggttcaggcagaggcccggcaaggagagggagggcgtggccaggatcaacggcctgggcggcgtgaagaccgcctacgccgacagcgtgaaggacaggttcaccatcagcagggacaacgccgagaacaccgtgtacctgcagatgaacagcctgaagcccgaggacaccgccatctactactgcgccgccaagttcagccccggctactgcggcggcagctggagcaacttcggctactggggccagggcacccaggttactgtgagctctggcgcgccagaggcagctgcaaaggaggcagctgcaaaggaggcagctgcaaaggaggcagctgcaaagggatccatggcagactctttgaataaccttgccaacatcaaacgggaacagggcaacattgaagaggcagttcgcctgtatcgcaaagcattagaagtcttcccagagtttgctgctgcacattccaatttagcaagtgtactgcaacagcagggcaagctgcaggaagcactgatgcactataaagaagccatacgaattagtcctacatttgctgatgcttattccaatatgggaaacactctaaaggagatgcaggatgtgcagggcgctttgcagtgttatactcgtgccatccagattaatcctgcctttgctgatgcacacagcaatctggcctccattcacaaggattcagggaatatcccagaagcaatagcttcttaccgcacagctctgaaacttaagcctgactttcctgatgcttattgtaacttggctcattgcctacagattgtctgtgattggacagactatgatgagcggatgaagaaattggttagtattgtagctgagcagctagagaagaatagactgccttctgtccatcctcaccatagcatgctgtaccctctttcccatggcttcaggaaggctattgcagagaggcatgggaatctctgcttggataagattaatgtccttcataaaccaccatatgaacatccaaaagacttgaagctcagtgatggccgattgcgtgtaggctatgtgagttctgacttcgggaatcaccctacttcacaccttatgcagtctattccaggcatgcataatcctgataagtttgaggtattctgctatgccttgagcccggatgatggtacaaactttcgagtgaaggtgatggcggaagccaatcatttcattgatctttctcagattccttgtaatggaaaagcagccgaccgcatccaccaagatggaattcacatccttgtgaatatgaatgggtataccaagggtgctcggaatgagctctttgctcttaggccagctcctattcaggccatgtggctgggctaccctgggactagtggtgcactgttcatggattacatcatcactgatcaggaaacttccccagctgaagttgcagagcagtattctgagaaactggcttatatgccccatactttctttattggtgatcatgctaatatgttccctcacctgaagaaaaaagcagtcatcgattttaaatccaatgggcacatttatgataatcggatagttctgaatggcatcgatctcaaagcatttctcgatagcctacccgatgtgaagattgtcaagatgaaatgtcctgatggaggtgacaatccagacagcagtaacacagctcttaatatgcccgttattcccatgaatacgattgcagaagcagtaattgaaatgattaacagagggcagattcagataacaattaacggattcagtattagcaatggactggcgactacacagattaataataaggctgcaaccggagaggaagttccccgtaccattattgtaaccacccgttcccagtatgggctaccagaagatgccattgtgtactgtaactttaatcagttatataaaattgacccatctaccctgcagatgtgggcaaatattctgaaacgtgtgcctaacagcgtgctttggctgttgcgttttccagcagtaggagaacccaatattcaacaatatgcacaaaatatgggccttccccagaaccgtatcattttctcacctgtggctcctaaagaggagcatgtcaggagaggtcagctggctgatgtctgcctggatactcctttgtgtaatggacacaccacagggatggatgttctctgggcaggaacacccatggtgactatgccaggagagactcttgcctctcgagttgcagcttctcagcttacttgtctaggatgtctcgagctcattgctaaaagcagacaggaatatgaagacatagctgtgaaactgggaaccgatctagaatacctgaagaaaattcgtggcaaagtctggaaacagagaatatctagccctctgttcaacaccaaacaatacacaatggaattagagcgactttatctgcagatgtgggagcattatgcagctggcaacaaacctgaccacatgattaagcctgttgaagtcaccgagtcagcctaagcggccgctcgagtctagagggcccgtttaaacccgctgatcagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 36 - protein, 886 aa, Artificial Sequence
  Synthetic Polypeptide
MAYPYDVPDYAAIAMGQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSGAPEAAAKEAAAKEAAAKEAAAKGSMADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVAEQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNPDSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKSRQEYEDIAVKLGTDLEYLKKIRGKVWKQRISSPLFNTKQYTMELERLYLQMWEHYAAGNKPDHMIKPVEVTESA

SEQ ID NO: 37 - DNA, 9000 bp, Artificial Sequence
  Synthetic Polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggcgagcgtttaagcttgagcaatggcatacccatacgatgttccagattacgctgcgatcgcaatgggccagctggtggagagcggcggcggcagcgtgcaggccggcggcagcctgaggctgagctgcgccgccagcggcatcgacagcagcagctactgcatgggctggttcaggcagaggcccggcaaggagagggagggcgtggccaggatcaacggcctgggcggcgtgaagaccgcctacgccgacagcgtgaaggacaggttcaccatcagcagggacaacgccgagaacaccgtgtacctgcagatgaacagcctgaagcccgaggacaccgccatctactactgcgccgccaagttcagccccggctactgcggcggcagctggagcaacttcggctactggggccagggcacccaggttactgtgagctctggcgcgccagaggcagctgcaaaggaggcagctgcaaaggaggcagctgcaaaggaggcagctgcaaagggatccatggcgtcttccgtgggcaacgtggccgacagtacagaaccaacgaaacgtatgctttccttccaagggttagctgagttggcacatcgagaatatcaggcaggagattttgaggcagctgagagacactgcatgcagctctggagacaagagcctgacaatactggtgttcttttattactttcatctatacacttccagtgtcgaaggctggacagatctgctcattttagcaccttggcaattaaacagaatccccttctagcagaagcctattcgaatttaggaaatgtgtacaaggaaagagggcagttgcaggaagcaatcgagcattatcgacatgccttgcggctgaagcctgatttcattgatggttatattaacctggcagcagccttggtagcagcaggtgacatggaaggagcagtacaagcctatgtctctgctcttcagtacaatcctgatttgtactgtgttcgcagtgacctggggaacctgctcaaagccctgggtcgcttggaagaagccaaggcatgttatttgaaagcaattgagacgcaaccaaactttgcagtagcctggagtaatctcggctgtgttttcaatgcacaaggggagatttggctggctattcatcactttgaaaaggctgtcacccttgacccaaattttctggatgcttatatcaatttaggaaatgtcttgaaagaggcacgcatttttgacagagctgtcgcagcttatcttcgtgccttaagtttgagcccaaatcatgcggtggtgcacggcaacctggcttgtgtgtactacgagcaaggcctaatagacctggccattgatacctacaggagagctatcgaactgcaaccccatttccccgatgcttactgcaacctagcaaatgctctcaaagagaagggcagtgttgctgaagcagaagactgttataacacagctcttcgtctgtgtcctactcatgcagactctttgaataaccttgccaacatcaaacgggaacagggcaacattgaagaggcagttcgcctgtatcgcaaagcattagaagtcttcccagagtttgctgctgcacattccaatttagcaagtgtactgcaacagcagggcaagctgcaggaagcactgatgcactataaagaagccatacgaattagtcctacatttgctgatgcttattccaatatgggaaacactctaaaggagatgcaggatgtgcagggcgctttgcagtgttatactcgtgccatccagattaatcctgcctttgctgatgcacacagcaatctggcctccattcacaaggattcagggaatatcccagaagcaatagcttcttaccgcacagctctgaaacttaagcctgactttcctgatgcttattgtaacttggctcattgcctacagattgtctgtgattggacagactatgatgagcggatgaagaaattggttagtattgtagctgagcagctagagaagaatagactgccttctgtccatcctcaccatagcatgctgtaccctctttcccatggcttcaggaaggctattgcagagaggcatgggaatctctgcttggataagattaatgtccttcataaaccaccatatgaacatccaaaagacttgaagctcagtgatggccgattgcgtgtaggctatgtgagttctgacttcgggaatcaccctacttcacaccttatgcagtctattccaggcatgcataatcctgataagtttgaggtattctgctatgccttgagcccggatgatggtacaaactttcgagtgaaggtgatggcggaagccaatcatttcattgatctttctcagattccttgtaatggaaaagcagccgaccgcatccaccaagatggaattcacatccttgtgaatatgaatgggtataccaagggtgctcggaatgagctctttgctcttaggccagctcctattcaggccatgtggctgggctaccctgggactagtggtgcactgttcatggattacatcatcactgatcaggaaacttccccagctgaagttgcagagcagtattctgagaaactggcttatatgccccatactttctttattggtgatcatgctaatatgttccctcacctgaagaaaaaagcagtcatcgattttaaatccaatgggcacatttatgataatcggatagttctgaatggcatcgatctcaaagcatttctcgatagcctacccgatgtgaagattgtcaagatgaaatgtcctgatggaggtgacaatccagacagcagtaacacagctcttaatatgcccgttattcccatgaatacgattgcagaagcagtaattgaaatgattaacagagggcagattcagataacaattaacggattcagtattagcaatggactggcgactacacagattaataataaggctgcaaccggagaggaagttccccgtaccattattgtaaccacccgttcccagtatgggctaccagaagatgccattgtgtactgtaactttaatcagttatataaaattgacccatctaccctgcagatgtgggcaaatattctgaaacgtgtgcctaacagcgtgctttggctgttgcgttttccagcagtaggagaacccaatattcaacaatatgcacaaaatatgggccttccccagaaccgtatcattttctcacctgtggctcctaaagaggagcatgtcaggagaggtcagctggctgatgtctgcctggatactcctttgtgtaatggacacaccacagggatggatgttctctgggcaggaacacccatggtgactatgccaggagagactcttgcctctcgagttgcagcttctcagcttacttgtctaggatgtctcgagctcattgctaaaagcagacaggaatatgaagacatagctgtgaaactgggaaccgatctagaatacctgaagaaaattcgtggcaaagtctggaaacagagaatatctagccctctgttcaacaccaaacaatacacaatggaattagagcgactttatctgcagatgtgggagcattatgcagctggcaacaaacctgaccacatgattaagcctgttgaagtcaccgagtcagcctaagcggccgctcgagtctagagggcccgtttaaacccgctgatcagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 38 - protein, 1211 aa, Artificial Sequence
  Synthetic Polypeptide
MAYPYDVPDYAAIAMGQLVESGGGSVQAGGSLRLSCAASGIDSSSYCMGWFRQRPGKEREGVARINGLGGVKTAYADSVKDRFTISRDNAENTVYLQMNSLKPEDTAIYYCAAKFSPGYCGGSWSNFGYWGQGTQVTVSSGAPEAAAKEAAAKEAAAKEAAAKGSMASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVAEQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNPDSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKSRQEYEDIAVKLGTDLEYLKKIRGKVWKQRISSPLFNTKQYTMELERLYLQMWEHYAAGNKPDHMIKPVEVTESA

SEQ ID NO: 39 - DNA, 7989 bp, Artificial Sequence
  Synthetic Polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggcgagcgtttaagcttgagcaatggcatacccatacgatgttccagattacgctgcgatcgcacaggtgcagctggtggagtctggaggagctctggtgcagcctggaggaagcctgcgcctgagctgtgcagctagcggatttcctgtgaaccgctacagcatgcgctggtaccgccaggctcctggtaaagagcgcgagtgggtggctggaatgagcagcgctggagatcgcagcagctacgaggacagcgtgaaaggacgctttacaatcagccgcgatgatgctcgcaacacagtgtacctgcagatgaactctctgaaacctgaggacactgctgtgtactactgtaacgtgaacgtgggtttcgagtactggggacagggaacacaggtgacagtgagctctggcgcgccagaggcagctgcaaaggaggcagctgcaaaggaggcagctgcaaaggaggcagctgcaaagggatccatggcagactctttgaataaccttgccaacatcaaacgggaacagggcaacattgaagaggcagttcgcctgtatcgcaaagcattagaagtcttcccagagtttgctgctgcacattccaatttagcaagtgtactgcaacagcagggcaagctgcaggaagcactgatgcactataaagaagccatacgaattagtcctacatttgctgatgcttattccaatatgggaaacactctaaaggagatgcaggatgtgcagggcgctttgcagtgttatactcgtgccatccagattaatcctgcctttgctgatgcacacagcaatctggcctccattcacaaggattcagggaatatcccagaagcaatagcttcttaccgcacagctctgaaacttaagcctgactttcctgatgcttattgtaacttggctcattgcctacagattgtctgtgattggacagactatgatgagcggatgaagaaattggttagtattgtagctgagcagctagagaagaatagactgccttctgtccatcctcaccatagcatgctgtaccctctttcccatggcttcaggaaggctattgcagagaggcatgggaatctctgcttggataagattaatgtccttcataaaccaccatatgaacatccaaaagacttgaagctcagtgatggccgattgcgtgtaggctatgtgagttctgacttcgggaatcaccctacttcacaccttatgcagtctattccaggcatgcataatcctgataagtttgaggtattctgctatgccttgagcccggatgatggtacaaactttcgagtgaaggtgatggcggaagccaatcatttcattgatctttctcagattccttgtaatggaaaagcagccgaccgcatccaccaagatggaattcacatccttgtgaatatgaatgggtataccaagggtgctcggaatgagctctttgctcttaggccagctcctattcaggccatgtggctgggctaccctgggactagtggtgcactgttcatggattacatcatcactgatcaggaaacttccccagctgaagttgcagagcagtattctgagaaactggcttatatgccccatactttctttattggtgatcatgctaatatgttccctcacctgaagaaaaaagcagtcatcgattttaaatccaatgggcacatttatgataatcggatagttctgaatggcatcgatctcaaagcatttctcgatagcctacccgatgtgaagattgtcaagatgaaatgtcctgatggaggtgacaatccagacagcagtaacacagctcttaatatgcccgttattcccatgaatacgattgcagaagcagtaattgaaatgattaacagagggcagattcagataacaattaacggattcagtattagcaatggactggcgactacacagattaataataaggctgcaaccggagaggaagttccccgtaccattattgtaaccacccgctcccagtatgggctaccagaagatgccattgtgtactgtaactttaatcagttatataaaattgacccatctaccctgcagatgtgggcaaatattctgaaacgtgtgcctaacagcgtgctttggctgttgcgttttccagcagtaggagaacccaatattcaacaatatgcacaaaatatgggccttccccagaaccgtatcattttctcacctgtggctcctaaagaggagcatgtcaggagaggtcagctggctgatgtctgcctggatactcctttgtgtaatggacacaccacagggatggatgttctctgggcaggaacacccatggtgactatgccaggagagactcttgcctctcgagttgcagcttctcagcttacttgtctaggatgtctcgagctcattgctaaaagcagacaggaatatgaagacatagctgtgaaactgggaaccgatctagaatacctgaagaaaattcgtggcaaagtctggaaacagagaatatctagccctctgttcaacaccaaacaatacacaatggaattagagcgactttatctgcagatgtgggagcattatgcagctggcaacaaacctgaccacatgattaagcctgttgaagtcaccgagtcagccgcggccgctcgagtctagagggcccgtttaaacccgctgatcagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 40 - protein, 880 aa, Artificial Sequence
  Synthetic Polypeptide
MAYPYDVPDYAAIAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGAPEAAAKEAAAKEAAAKEAAAKGSMADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVAEQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNPDSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKSRQEYEDIAVKLGTDLEYLKKIRGKVWKQRISSPLFNTKQYTMELERLYLQMWEHYAAGNKPDHMIKPVEVTESAAAARV

SEQ ID NO: 41 - DNA, 8967 bp, Artificial Sequence
  Synthetic Polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggcgagcgtttaagcttgagcaatggcatacccatacgatgttccagattacgctgcgatcgcacaggtgcagctggtggagtctggaggagctctggtgcagcctggaggaagcctgcgcctgagctgtgcagctagcggatttcctgtgaaccgctacagcatgcgctggtaccgccaggctcctggtaaagagcgcgagtgggtggctggaatgagcagcgctggagatcgcagcagctacgaggacagcgtgaaaggacgctttacaatcagccgcgatgatgctcgcaacacagtgtacctgcagatgaactctctgaaacctgaggacactgctgtgtactactgtaacgtgaacgtgggtttcgagtactggggacagggaacacaggtgacagtgagctctggcgcgccagaggcagctgcaaaggaggcagctgcaaaggaggcagctgcaaaggaggcagctgcaaagggatccatggcgtcttccgtgggcaacgtggccgacagtacagaaccaacgaaacgtatgctttccttccaagggttagctgagttggcacatcgagaatatcaggcaggagattttgaggcagctgagagacactgcatgcagctctggagacaagagcctgacaatactggtgttcttttattactttcatctatacacttccagtgtcgaaggctggacagatctgctcattttagcaccttggcaattaaacagaatccccttctagcagaagcctattcgaatttaggaaatgtgtacaaggaaagagggcagttgcaggaagcaatcgagcattatcgacatgccttgcggctgaagcctgatttcattgatggttatattaacctggcagcagccttggtagcagcaggtgacatggaaggagcagtacaagcctatgtctctgctcttcagtacaatcctgatttgtactgtgttcgcagtgacctggggaacctgctcaaagccctgggtcgcttggaagaagccaaggcatgttatttgaaagcaattgagacgcaaccaaactttgcagtagcctggagtaatctcggctgtgttttcaatgcacaaggggagatttggctggctattcatcactttgaaaaggctgtcacccttgacccaaattttctggatgcttatatcaatttaggaaatgtcttgaaagaggcacgcatttttgacagagctgtcgcagcttatcttcgtgccttaagtttgagcccaaatcatgcggtggtgcacggcaacctggcttgtgtgtactacgagcaaggcctaatagacctggccattgatacctacaggagagctatcgaactgcaaccccatttccccgatgcttactgcaacctagcaaatgctctcaaagagaagggcagtgttgctgaagcagaagactgttataacacagctcttcgtctgtgtcctactcatgcagactctttgaataaccttgccaacatcaaacgggaacagggcaacattgaagaggcagttcgcctgtatcgcaaagcattagaagtcttcccagagtttgctgctgcacattccaatttagcaagtgtactgcaacagcagggcaagctgcaggaagcactgatgcactataaagaagccatacgaattagtcctacatttgctgatgcttattccaatatgggaaacactctaaaggagatgcaggatgtgcagggcgctttgcagtgttatactcgtgccatccagattaatcctgcctttgctgatgcacacagcaatctggcctccattcacaaggattcagggaatatcccagaagcaatagcttcttaccgcacagctctgaaacttaagcctgactttcctgatgcttattgtaacttggctcattgcctacagattgtctgtgattggacagactatgatgagcggatgaagaaattggttagtattgtagctgagcagctagagaagaatagactgccttctgtccatcctcaccatagcatgctgtaccctctttcccatggcttcaggaaggctattgcagagaggcatgggaatctctgcttggataagattaatgtccttcataaaccaccatatgaacatccaaaagacttgaagctcagtgatggccgattgcgtgtaggctatgtgagttctgacttcgggaatcaccctacttcacaccttatgcagtctattccaggcatgcataatcctgataagtttgaggtattctgctatgccttgagcccggatgatggtacaaactttcgagtgaaggtgatggcggaagccaatcatttcattgatctttctcagattccttgtaatggaaaagcagccgaccgcatccaccaagatggaattcacatccttgtgaatatgaatgggtataccaagggtgctcggaatgagctctttgctcttaggccagctcctattcaggccatgtggctgggctaccctgggactagtggtgcactgttcatggattacatcatcactgatcaggaaacttccccagctgaagttgcagagcagtattctgagaaactggcttatatgccccatactttctttattggtgatcatgctaatatgttccctcacctgaagaaaaaagcagtcatcgattttaaatccaatgggcacatttatgataatcggatagttctgaatggcatcgatctcaaagcatttctcgatagcctacccgatgtgaagattgtcaagatgaaatgtcctgatggaggtgacaatccagacagcagtaacacagctcttaatatgcccgttattcccatgaatacgattgcagaagcagtaattgaaatgattaacagagggcagattcagataacaattaacggattcagtattagcaatggactggcgactacacagattaataataaggctgcaaccggagaggaagttccccgtaccattattgtaaccacccgttcccagtatgggctaccagaagatgccattgtgtactgtaactttaatcagttatataaaattgacccatctaccctgcagatgtgggcaaatattctgaaacgtgtgcctaacagcgtgctttggctgttgcgttttccagcagtaggagaacccaatattcaacaatatgcacaaaatatgggccttccccagaaccgtatcattttctcacctgtggctcctaaagaggagcatgtcaggagaggtcagctggctgatgtctgcctggatactcctttgtgtaatggacacaccacagggatggatgttctctgggcaggaacacccatggtgactatgccaggagagactcttgcctctcgagttgcagcttctcagcttacttgtctaggatgtctcgagctcattgctaaaagcagacaggaatatgaagacatagctgtgaaactgggaaccgatctagaatacctgaagaaaattcgtggcaaagtctggaaacagagaatatctagccctctgttcaacaccaaacaatacacaatggaattagagcgactttatctgcagatgtgggagcattatgcagctggcaacaaacctgaccacatgattaagcctgttgaagtcaccgagtcagcctaagcggccgctcgagtctagagggcccgtttaaacccgctgatcagccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 42 - protein, 1200 aa, Artificial Sequence
  Synthetic Polypeptide
MAYPYDVPDYAAIAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGAPEAAAKEAAAKEAAAKEAAAKGSMASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVAEQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNPDSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKSRQEYEDIAVKLGTDLEYLKKIRGKVWKQRISSPLFNTKQYTMELERLYLQMWEHYAAGNKPDHMIKPVEVTESA

SEQ ID NO: 43 - protein, 5 aa, Artificial Sequence
  Synthetic Polypeptide
EAAAK